SEQUENCE LISTING (44 sequences)

SEQ ID NO: 1 - protein, 387 aa, Artificial Sequence
  YopE1-138 - human RIG1 CARD domains1-245
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCPPSEVSDTNLYSPFKPRN

SEQ ID NO: 2 - protein, 370 aa, Artificial Sequence
  YopE1-138 - human RIG1 CARD domains1-228
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDPECQNLSENSCP

SEQ ID NO: 3 - protein, 359 aa, Artificial Sequence
  YopE1-138 -  human RIG1 CARD domains1-217
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTTEQRRSLQAFQDYIRKTLDPTYILSYMAPWFREEEVQYIQAEKNNKGPMEAATLFLKFLLELQEEGWFRGFLDALDHAGYSGLYEAIESWDFKKIEKLEEYRLLLKRLQPEFKTRIIPTDIISDLSECLINQECEEILQICSTKGMMAGAEKLVECLLRSDKENWPKTLKLALEKERNKFSELWIVEKGIKDVETEDLEDKMETSDIQIFYQEDP

SEQ ID NO: 4 - protein, 388 aa, Artificial Sequence
  YopE1-138 -  murine RIG1 CARD domains1-246
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTAEQRQNLQAFRDYIKKILDPTYILSYMSSWLEDEEVQYIQAEKNNKGPMEAASLFLQYLLKLQSEGWFQAFLDALYHAGYCGLCEAIESWDFQKIEKLEEHRLLLRRLEPEFKATVDPNDILSELSECLINQECEEIRQIRDTKGRMAGAEKMAECLIRSDKENWPKVLQLALEKDNSKFSELWIVDKGFKRAESKADEDDGAEASSIQIFIQEEPECQNLSQNPGPPSEASSNNLHSPLKPRN

SEQ ID NO: 5 - protein, 371 aa, Artificial Sequence
  YopE1-138 - murine RIG1 CARD domains1-229
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTAEQRQNLQAFRDYIKKILDPTYILSYMSSWLEDEEVQYIQAEKNNKGPMEAASLFLQYLLKLQSEGWFQAFLDALYHAGYCGLCEAIESWDFQKIEKLEEHRLLLRRLEPEFKATVDPNDILSELSECLINQECEEIRQIRDTKGRMAGAEKMAECLIRSDKENWPKVLQLALEKDNSKFSELWIVDKGFKRAESKADEDDGAEASSIQIFIQEEPECQNLSQNPGP

SEQ ID NO: 6 - protein, 360 aa, Artificial Sequence
  YopE1-138 - murine RIG1 CARD domains1-218
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTAEQRQNLQAFRDYIKKILDPTYILSYMSSWLEDEEVQYIQAEKNNKGPMEAASLFLQYLLKLQSEGWFQAFLDALYHAGYCGLCEAIESWDFQKIEKLEEHRLLLRRLEPEFKATVDPNDILSELSECLINQECEEIRQIRDTKGRMAGAEKMAECLIRSDKENWPKVLQLALEKDNSKFSELWIVDKGFKRAESKADEDDGAEASSIQIFIQEEP

SEQ ID NO: 7 - protein, 242 aa, Artificial Sequence
  YopE1-138 - human MAVS CARD domain1-100
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMPFAEDKTYKYICRNFSNFCNVDVVEILPYLPCLTARDQDRLRATCTLSGNRDTLWHLFNTLQRRPGWVEYFIAALRGCELVDLADEVASVYESYQPRTS

SEQ ID NO: 8 - protein, 243 aa, Artificial Sequence
  YopE1-138 - murine MAVS CARD domain1-101
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMTFAEDKTYKYIRDNHSKFCCVDVLEILPYLSCLTASDQDRLRASYRQIGNRDTLWGLFNNLQRRPGWVEVFIRALQICELPGLADQVTRVYQSYLPPGTS

SEQ ID NO: 9 - protein, 564 aa, Artificial Sequence
  YopE1-138 - N. vectensis cGAS
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMATLERLLDLLREYHLDDVLFHNSTPELGIQHRSRPKQKRIIRGKKQQKSKKLKRNEQQQPFPKGDLETLRRFSVTDVKISKQSTKWAKKMADKHLEIIRKHCKTNSIKLFNHFEYTGSFYEHLKTIDADELDIMVALSIKMDELEVEQVTPGYAGLKLRDTPSNRNKYNDLTIADNYGRYLSPEKVSRWFFSLVQKAVNTYKDEIPQTEVKLTDNGPATTLVITYREGDKPQEKNRRLSIDLVPALLFKDKTKPAGDDLRAWHYVAKTIPKGARLKEPLPFRSELLWRQSFSLKEKHLMDKLDKDDNGCRREMVRIVKTIVKKDPTLAQLSSYHIKTAFLQYNFSDVKLDWEGKKLAERFLHFLEFLRDRVKDKTLNNYFITDLNLLDDLNDSNIDNIANRLDKIIQNETERAKIFTTQRQ

SEQ ID NO: 10 - protein, 504 aa, Artificial Sequence
  YopE1-138 - human cGAS161-522
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 11 - protein, 504 aa, Artificial Sequence
  YopE1-138 - murine cGAS146-507
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESREPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 12 - protein, 505 aa, Artificial Sequence
  YopE1-138 - N. vectensis cGAS60-422
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRQPFPKGDLETLRRFSVTDVKISKQSTKWAKKMADKHLEIIRKHCKTNSIKLFNHFEYTGSFYEHLKTIDADELDIMVALSIKMDELEVEQVTPGYAGLKLRDTPSNRNKYNDLTIADNYGRYLSPEKVSRWFFSLVQKAVNTYKDEIPQTEVKLTDNGPATTLVITYREGDKPQEKNRRLSIDLVPALLFKDKTKPAGDDLRAWHYVAKTIPKGARLKEPLPFRSELLWRQSFSLKEKHLMDKLDKDDNGCRREMVRIVKTIVKKDPTLAQLSSYHIKTAFLQYNFSDVKLDWEGKKLAERFLHFLEFLRDRVKDKTLNNYFITDLNLLDDLNDSNIDNIANRLDKIIQNETERAKIFTTQRQ

SEQ ID NO: 13 - protein, 436 aa, Artificial Sequence
  YopE1-138 -  murine MDA51-294
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMSIVCSAEDSFRNLILFFRPRLKMYIQVEPVLDHLIFLSAETKEQILKKINTCGNTSAAELLLSTLEQGQWPLGWTQMFVEALEHSGNPLAARYVKPTLTDLPSPSSETAHDECLHLLTLLQPTLVDKLLINDVLDTCFEKGLLTVEDRNRISAAGNSGNESGVRELLRRIVQKENWFSTFLDVLRQTGNDALFQELTGGGCPEDNTDLANSSHRDGPAANECLLPAVDESSLETEAWNVDDILPEASCTDSSVTTESDTSLAEGSVSCFDESLGHNSNMGRDSGTMGSDSDES

SEQ ID NO: 14 - protein, 373 aa, Artificial Sequence
  YopE1-138 - murine MDA51-231
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMSIVCSAEDSFRNLILFFRPRLKMYIQVEPVLDHLIFLSAETKEQILKKINTCGNTSAAELLLSTLEQGQWPLGWTQMFVEALEHSGNPLAARYVKPTLTDLPSPSSETAHDECLHLLTLLQPTLVDKLLINDVLDTCFEKGLLTVEDRNRISAAGNSGNESGVRELLRRIVQKENWFSTFLDVLRQTGNDALFQELTGGGCPEDNTDLANSSHRDGPAANECLLPAVDES

SEQ ID NO: 15 - protein, 436 aa, Artificial Sequence
  YopE1-138 - human MDA51-294
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQPNLEKEVWGMENNSSESSFADSSVVSESDTSLAEGSVSCLDESLGHNSNMGSDSGTMGSDSDEE

SEQ ID NO: 16 - protein, 373 aa, Artificial Sequence
  YopE1-138 - human MDA51-231
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFATLESRMSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIENLSQVDGPQVEEQLLSTTVQP

SEQ ID NO: 17 - protein, 20 aa, Homo sapiens
EDIIRNIARHLAQVGDSMDR

SEQ ID NO: 18 - protein, 15 aa, Homo sapiens
IARHLAQVGDSMDRS

SEQ ID NO: 19 - protein, 20 aa, Mus musculus
EEIIHNIARHLAQIGDEMDH

SEQ ID NO: 20 - protein, 14 aa, Mus musculus
IARHLAQIGDEMDH

SEQ ID NO: 21 - protein, 17 aa, Homo sapiens
KKLSECLKRIGDELDSN

SEQ ID NO: 22 - protein, 14 aa, Homo sapiens
LSECLKRIGDELDS

SEQ ID NO: 23 - protein, 17 aa, Mus musculus
KKLSECLRRIGDELDSN

SEQ ID NO: 24 - protein, 14 aa, Mus musculus
LSECLRRIGDELDS

SEQ ID NO: 25 - protein, 138 aa, Yersinia enterocolitica
MKISSFISTSLPLPASVSGSSSVGEMSGRSVSQQKSDQYANNLAGRTESPQGSSLASRIIERLSSMAHSVIGFIQRMFSEGSHKPVVTPALTPAQMPSPTSFSDSIKQLAAETLPKYMQQLSSLDAETLQKNHDQFAT

SEQ ID NO: 26 - protein, 81 aa, Salmonella enterica
VTKITLSPQNFRIQKQETTLLKEKSTEKNSLAKSILAVKNHFIELRSKLSERFISHKNTESSATHFHRGSASEGRAVLTNK

SEQ ID NO: 27 - protein, 105 aa, Salmonella enterica
VTKITLSPQNFRIQKQETTLLKEKSTEKNSLAKSILAVKNHFIELRSKLSERFISHKNTESSATHFHRGSASEGRAVLTNKVVKDFMLQTLNDIDIRGSASKDPA

SEQ ID NO: 28 - protein, 210 aa, Salmonella enterica
MPYTSVSTYARALSGNKLPHVAAGDYENKLSTKIMKGILYVLTAGLAYGFTRVIEHYCNVTPKVAEFCANAGNIHNHLADAVRDGLFTIDVELSDGRMLTFEQLSLIAEGKPIVRISDGEHTVEVEGTFEEICMRLEEGFFEAPAYYDYDIDEKYKTVRERMAAYNALPQALGAIPCLEYYIARASNMQEAKAQWAADIKARYHNYLDNY

SEQ ID NO: 29 - DNA, 629 bp, Artificial Sequence
  pBR322 ori
tagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 30 - DNA, 629 bp, Artificial Sequence
  ColE1 ori
tagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcc

SEQ ID NO: 31 - DNA, 225 bp, Artificial Sequence
  rop
atgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctga

SEQ ID NO: 32 - DNA, 45 bp, Artificial Sequence
  Primer-T3T_887
cacatgtggtcgacgaatagacagcgaaagttgttgaaataattg

SEQ ID NO: 33 - DNA, 47 bp, Artificial Sequence
  Primer-T3T_955
cactacccccttgtttttatccatattaattgcgcggtttaaacggg

SEQ ID NO: 34 - DNA, 25 bp, Artificial Sequence
  Primer - T3T_956
tatggataaaaacaagggggtagtg

SEQ ID NO: 35 - DNA, 44 bp, Artificial Sequence
  Primer - T3T_888
catgcgaatgggcccgttttcagtataaaaagcacggtatatac

SEQ ID NO: 36 - DNA, 138 bp, Artificial Sequence
  Multiple cloning site directly following the yopE1-138 fragment on pBad_Si2 plasmid
gttcgccacgctcgagtctagattcgaaaagcttgggcccgaacaaaaactcatctcagaagaggatctgaatagcgccgtcgaccatcatcatcatcatcattgagtttaaacggtctccagcttggctgttttggc

SEQ ID NO: 37 - protein, 5 aa, Artificial Sequence
  Amino acid motif recognized by Enterokinase (light chain) / Enteropeptidase
DDDDK

SEQ ID NO: 38 - protein, 8 aa, Artificial Sequence
  Amino acid motif recognized by PreScission Protease/human Rhinovirusprotease (HRV 3C)
LEVLFQGP

SEQ ID NO: 39 - protein, 7 aa, Artificial Sequence
  Amino acid motif recognized by TEV protease (tobacco etch virus)
ENLYFQS

SEQ ID NO: 40 - protein, 7 aa, Artificial Sequence
  Modified amino acid motifs based on the Glu-X-X-Tyr-X-Gln/Gly or Ser (where X is any amino acid) recognized by TEV protease (tobacco etch virus)
  VARIANT (2)..(2): Xaa can be any naturally occurring amino acid
  VARIANT (3)..(3): Xaa can be any naturally occurring amino acid
  VARIANT (5)..(5): Xaa can be any naturally occurring amino acid
  VARIANT (7)..(7): Xaa can be serine or glycine
EXXYXQX

SEQ ID NO: 41 - protein, 7 aa, Artificial Sequence
  Amino acid motif recognized by TVMV protease
ETVRFQS

SEQ ID NO: 42 - protein, 4 aa, Artificial Sequence
  Amino acid motif recognized by FactorXa protease
  VARIANT (2)..(2): Xaa can be glutamic acid or aspartic acid
IXGR

SEQ ID NO: 43 - protein, 6 aa, Artificial Sequence
  Amino acid motif recognized by Thrombin
LVPRGS

SEQ ID NO: 44 - protein, 8 aa, Simian virus 40
PPKKKRKV

(19) United States
(12) Patent Application Publication
(10) Pub. No.: US 2024/0165169 A1
(43) Pub. Date: May 23, 2024

(54) Pharmaceutical Combinations for Treating Cancer

(71) Applicants: T3 PHARMACEUTICALS AG, Allschwil (CH); BOEHRINGER INGELHEIM INTERNATIONAL GMBH, Ingelheim am Rhein (DE)

(72) Inventors: Simon ITTIG, Bottmingen (CH); Christoph KASPER, Olten (CH); Falk SAUPE, Basel (CH); Marlise AMSTUTZ, Basel (CH); Mélodie DUVAL, Huninque (FR); Irene WAIZENEGGER, Ingelheim am Rhein (DE)

(21) Appl. No.: 18/551,553

(22) PCT Filed: Mar. 24, 2022
(86) PCT No.: PCT/EP2022/057750

§ 371 Date: Sep. 20, 2023

(30) Foreign Application Priority Data
Mar. 25, 2021 (EP) 21164883.7

(51) Int. Cl. A61K 35/74; A61K 35/00; A61K 39/00; A61K 39/395; A61P 35/00

(52) U.S. Cl. CPC A61K 35/74; A61K 39/3955; A61P 35/00; A61K 2035/11; A61K 2039/505

(57) ABSTRACT

The present invention relates to a pharmaceutical combination comprising a recombinant Gram-negative bacterial strain and an immune checkpoint modulator (ICM) and their use in a method for the prevention, delay of progression or treatment of cancer in a subject.

### FIELD OF THE INVENTION

The present invention relates to a pharmaceutical combination comprising a recombinant Gram-negative bacterial strain and an immune checkpoint modulator (ICM) and their use in a method for the prevention, delay of progression or treatment of cancer in a subject.

### BACKGROUND OF THE INVENTION

Despite the ever-increasing number of cancer therapies in general, and combination cancer therapies in particular, cancer is still the third most common cause of death worldwide after cardiovascular diseases and infectious/parasitic diseases; in absolute numbers, this corresponds to 7.6 million deaths (ca. 13% of all deaths) in any given year. The WHO estimates deaths due to cancer to increase to 13.1 million by 2030. These statistics illustrate the fact that cancer remains a critical health condition and that there is an urgent need for new treatments. In a recent approach, immune checkpoint inhibitors (CPIs) have been used for the treatment of cancer. In the last six years, four engineered monoclonal antibody immune checkpoint inhibitor agents have been approved in more than 50 global markets for six forms of cancer; ipilimumab (anti-CTLA-4), pembrolizumab and nivolumab (anti-PD-1), and atezolizumab (anti-PD-L1), with response rates of up to 40-50% with PD-1-based therapy. Treatment with current checkpoint inhibitor monotherapy, however, is not effective in all cancer types, as tumours with lower mutational burden and/or lower immunogenicity may be inherently resistant to this form of therapy. CPIs, which are generally considered very effective immunomodulatory agents, produce relatively low response rates in many tumor types (<20% on average; (Carretero-Gonzalez et al., 2018)), and certain cancers don't seem to respond to them at all. Thus, when using checkpoint inhibitors there is a need to convert a proportion of patients destined not to benefit from single-agent checkpoint blockade into long-term survivors.

The rationale for combination therapy in cancer is usually to use drugs that work by different mechanisms, thereby decreasing the likelihood that resistant cancer cells will develop. On the other hand, administration of two or more drugs to treat a given condition, such as cancer, generally raises a number of potential problems due to complex in vivo interactions between drugs. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug. Thus, when two drugs are administered to treat the same condition, it is unpredictable whether each will complement, have no effect on, or interfere with, the therapeutic activity of the other in a subject. Not only may the interaction between two drugs affect the intended therapeutic activity of each drug, but the interaction may increase the levels of toxic metabolites. The interaction may also heighten or lessen the side effects of each drug. Hence, upon administration of two drugs to treat a disease, it is unpredictable what change, either deterioration or improvement, will occur in the side effect profile of each drug. Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs. Therefore, the effects of a combination therapy of two or more drugs cannot be easily predicted.

### SUMMARY OF THE INVENTION

It has now unexpectedly been found that a combination comprising a recombinant Gram-negative bacterial strain encoding a heterologous protein and an antagonistic PD-1 antibody immune checkpoint modulator (ICM) is useful for the prevention, delay of progression or treatment of cancer. It was unexpectedly found that treatment with said combination provides a synergistic anti-tumor effect.

Taking these unexpected findings into account, the inventors herewith provide the present invention in its following aspects.

In a first aspect the present invention provides a pharmaceutical combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM), wherein the ICM is ezabenlimab; and optionally
(c) one or more pharmaceutically acceptable diluents, excipients or carriers.

In a second aspect the present invention provides a pharmaceutical combination as described herein, for use as a medicament.

In a third aspect the present invention provides a pharmaceutical combination as described herein, for use in a method for the prevention, delay of progression or treatment of cancer in a subject.

In a fourth aspect the present invention provides kit of parts comprising a first container, a second container and a package insert, wherein the first container comprises at least one dose of a medicament comprising a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter, the second container comprises at least one dose of a medicament comprising an immune checkpoint inhibitor (ICM), wherein the ICM is ezabenlimab, and the package insert optionally comprises instructions for treating a subject for cancer using the medicaments.

### BRIEF DESCRIPTION OF THE FIGURES

FIG. 1: The Yersinia enterocolitica pYV-MRS40 virulence plasmid, pYV. The 75′115 bp plasmid of Yersinia virulence (pYV) of strain MRS40 drawn to scale. Selected T3SS machinery, T3SS effector proteins, origin of replication and the arsenic resistance (encoded by genes arsC, B, R and H) are indicated:

I. replication origin 53 . . . 203; II. YopO 7377 . . . 9566; III. YopP 10311 . . . 11177; IV. YopQ 12920 . . . 13468; V. YopT 13989 . . . 14957; VI. sycT 14957 . . . 15349; VII. YopM 18926 . . . 20029; VIII. YopD 21283 . . . 22203; IX. YopB 22222 . . . 23427; X. sycD 23405 . . . 23911; XI. YopH 47882 . . . 49288; XII. sycH 49516 . . . 49941; XIII. sycE 51552 . . . 51944; XIV. YopE 52137 . . . 52796; XV. yadA 62215 . . . 63678; XVI. arsC 67164 . . . 67589; XVII. arsB 67602 . . . 68891; XVIII. arsR 68937 . . . 69257; XIX. arsH 69343 . . . 70041

FIG. 2: The modified Yersinia enterocolitica MRS40 virulence plasmid, pYV-Y004, The 71,408 bp pYV-Y004 drawn to scale. Selected T3SS machinery, disrupted T3SS effector proteins, origin of replication and the arsenic resistance genes (encoded by genes arsC, B, R and H) are indicated

I. replication origin 53 . . . 203; II. YopO disrupted 7409 . . . 8116; III. YopP disrupted 8597 . . . 8949; IV. YopQ 10692 . . . 11240; V. YopT disrupted 11761 . . . 12301; VI. sycT 12301 . . . 12693; VII. YopM disrupted 16270 . . . 17375; VIII. YopD 18629 . . . 19549; IX. YopB 19568 . . . 20773; X. sycD 20751 . . . 21257; XI. YopH disrupted 45213 . . . 45563; XII. sycH 45791 . . . 46216; XIII. sycE 47827 . . . 48219; XIV. YopE disrupted 48426 . . . 49089; XV. yadA 58508 . . . 59971; XVI. arsC 63457 . . . 63882; XVII. arsB 63895 . . . 65184; XVIII. arsR 65230 . . . 65550; XIX. arsH 65636 . . . 66334

FIG. 3: The modified Yersinia enterocolitica MRS40 virulence plasmid, pYV-Y051, encoding YopE₁₋₁₃₈-human cGAS₁₆₁₋₅₂₂ and YopE₁₋₁₃₈-human RIG-I CARD2 encoded on the endogenous pYV plasmid on the endogenous sites of yopH and yopE, respectively. The 73′073 bp pYV-051 drawn to scale. Selected T3SS machinery, disrupted T3SS effector proteins, cargo proteins (YopE₁₋₁₃₈ in frame with hRigI CARD2 domains and YopE₁₋₁₃₈ (codon changed) in frame with hcGAS₁₆₁₋₅₂₂), origin of replication and the arsenic resistance genes (encoded by genes arsC, B, R and H) are indicated:

I. replication origin 53 . . . 203; II. YopO disrupted 7409 . . . 8116; III. YopP disrupted 8597 . . . 8949; IV. YopQ 10692 . . . 11240; V. YopT disrupted 11761 . . . 12301; VI. sycT 12301 . . . 12693; VII. YopM disrupted 16270 . . . 17375; VIII. YopD 18629 . . . 19549; IX. YopB 19568 . . . 20773; X. sycD 20751 . . . 21257; XI. YopH::YopE₁₋₁₃₈ (codon adapted)-hcGAS₁₆₁₋₅₂₂; 45228 . . . 46742; XII. sycH 46970 . . . 47395; XIII. sycE 49006 . . . 49398; XIV. YopE::YopE₁₋₁₃₈-hRigI (CARD2) 49591 . . . 50754; XV. yadA 60173 . . . 61636; XVI. arsC 65122 . . . 65547; XVII. arsB 65560 . . . 66849; XVIII. arsR 66895 . . . 67215; XIX. arsH 67301 . . . 67999;

FIG. 4: Description of vector pBad_Si_2. Vector map of the cloning plasmid pBad_Si_2 (5,085 pb) used to generate fusion constructs with YopE₁₋₁₃₈. The chaperone SycE and the YopE₁₋₁₃₈-fusion are under the native Y. enterocolitica promoter.

I. araBAD promoter region 4 . . . 279; II. PBAD 250 . . . 277; III. MCS I 317 . . . 331; IV. sycE 339 . . . 731; V. YopE1-138 924 . . . 1337; VI. MCS II 1338 . . . 1361; VII. Myc-tag 1368 . . . 1397; VIII. 6×his-tag 1413 . . . 1430; IX. stop 1431 . . . 1433; X. rrnB 1536 . . . 1693; XI. rrnB T2 1834 . . . 1861; XII. AmpR 1972 . . . 2832; XIII. pBR322 origin 2981 . . . 3609; XIV. araC 4181 . . . 5059;

FIG. 5: Description of vector pT3P-454 Vector map of the medium-copy number cloning plasmid pT3P-454 (5,818 pb) encoding the fusion Rig1-CARD₂ (murine₁₋₂₄₆) with codon optimized YopE₁₋₁₃₈. The chaperone SycE and the YopE₁₋₁₃₈-fusion are under the native Y. enterocolitica promoter.

I. araBAD promoter region 4 . . . 279; II. PBAD 250 . . . 277; III. MCS I 317 . . . 331; IV. SycE 339 . . . 731; V. YopE1-138 924 . . . 1337; VI. Rig1-CARD-Domains that terminate with a stop codon 1349 . . . 2087; VII. Myc-tag 2101 . . . 2130; VIII. 6×his-tag 2146 . . . 2163; IX. Second stop 2164 . . . 2166; X. rrnB 2269 . . . 2426; XI. rrnB T2 2567 . . . 2594; XII. Ampicilline Resistance gene 2705 . . . 3565; XIII. pBR322 origin 3714 . . . 4342; XIV. araC 4914 . . . 5792

FIG. 6: Description of vector pT3P-453 Vector map of the medium-copy number cloning plasmid pT3P-453 (5,815 pb) encoding the fusion Rig1-CARD₂ (human₁₋₂₄₅) with codon optimized YopE₁₋₁₃₈. The chaperone SycE and the YopE₁₋₁₃₈-fusion are under the native Y. enterocolitica promoter.

I. araBAD promoter region 4 . . . 279; II. PBAD 250 . . . 277; III. MCS I 317 . . . 331; IV. SycE 339 . . . 731; V. YopE1-138 924 . . . 1337; VI. human Rig1-CARD-Domains that terminate with a stop codon 1350 . . . 2087; VII. Myc-tag 2098 . . . 2127; VIII. 6×his-tag 2143 . . . 2160; IX. Second stop 2161 . . . 2163; X. rrnB 2266 . . . 2423; XI. rrnB T2 2564 . . . 2591; XII. Ampicilline Resistance gene 2702 . . . 3562; XIII. pBR322 origin 3711 . . . 4339; XIV. araC 4911 . . . 5789

FIG. 7: Description of vector pT3P-715. Vector map of the medium-copy number cloning plasmid pT3P-715 (4,149 bp) used to generate fusion constructs with YopE₁₋₁₃₈. The chaperone SycE and the YopE₁₋₁₃₈-fusion are under the native Y. enterocolitica promoter. I. araBAD promoter region 4 . . . 279; II. PBAD 250 . . . 277; III. MCS I 317 . . . 331; IV. sycE 339 . . . 731; V. YopE1-138 924 . . . 1337; VI. MCS II 1338 . . . 1361; VII. Myc-tag 1368 . . . 1397; VIII. 6×his-tag 1413 . . . 1430; IX. stop 1431 . . . 1433; X. rrnB 1536 . . . 1693; XI. rrnB T2 1834 . . . 1861; XII. Chloramphenicol Resistance gene 2110 . . . 2766; XIII. pBR322 origin 2924 . . . 3552;

FIG. 8: Description of vector pT3P-751 encoding YopE₁₋₁₃₈-human cGAS₁₆₁₋₅₂₂ and YopE₁₋₁₃₈-human RIG-I CARD₂. Vector map of the medium-copy number vector pT3P-751 (6,407 bp) encoding YopE₁₋₁₃₈-human cGAS₁₆₁₋₅₂₂ and YopE₁₋₁₃₈-human RIG-I CARD2 in one operon under control of the yopE promoter I. araBAD promoter region 4 . . . 279; II. PBAD 250 . . . 277; III. MCS I 317 . . . 331; IV. SycE 339 . . . 731; V. YopE1-138 924 . . . 1337; VI. human Rig1-CARD-Domains that terminates with a stop codon 1350 . . . 2087; VII. yopE1-138 (codon changed) 2101 . . . 2514; VIII. h_cGAS161-522 that terminates with a stop codon 2527 . . . 3614; IX. Myc-tag 3626 . . . 3655; X. 6×his-tag 3671 . . . 3688; XI. Third stop 3689 . . . 3691; XII. rrnB 3794 . . . 3951; XIII. rrnB T2 4092 . . . 4119; XIV. Chloramphenicol Resistance gene 4368 . . . 5024; XV. pBR322 origin 5182 . . . 5810;

FIG. 9: Tumor progression in wildtype Balb/c mice allografted s.c. with EMT-6 breast cancer cells treated with either Y. enterocolitica ΔHOPEMT encoding YopE₁₋₁₃₈-murine RIG-I CARD₂, or with an anti-PD-1 antibody, or with a combination of both treatments. Wildtype Balb/c mice allografted s.c. with EMT-6 breast cancer cells were intratumorally (i.t.) injected with 7.5×10⁷ CFU of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-mRIG-I CARD₂ is encoded under control of the yopE promoter) or sterile PBS as a control. In combination, mice were intraperitoneally (i.p.) injected with anti-PD-1 antibody (10 mg/kg per injection) or sterile PBS as a control. Injections started once the tumor had reached a volume of 60-130 mm³ (average size of 92 mm³+/−19). The average tumor volume (n=15 animal per group) is indicated (I) as mm³. The day of the first treatment is defined as day 0. Tumor volume was measured over the following days (II: days) with calipers. i.t. treatments (III) were performed on d0, d1, d5, d6, d10 and d11 and i.p. treatments (IV) were performed on d0, d4, d7, and d11. Data are displayed until each group contained less than 50% of initial mice alive.

Groups were distributed as (shown as: i.t. treatment+i.p. treatment): V. PBS+PBS; VI. Y. enterocolitica ΔHOPEMT encoding YopE₁₋₁₃₈-murine RIG-I CARD₂+PBS; VII. PBS+anti-PD-1; VIII. Y. enterocolitica ΔHOPEMT encoding YopE₁₋₁₃₈-murine RIG-I CARD₂+anti-PD-1

FIG. 10: Tumor progression in wildtype Balb/c mice allografted s.c. with EMT-6 breast cancer cells of the control group. Wildtype Balb/c mice allografted s.c. with EMT-6 breast cancer cells were intratumorally (i.t.) injected with sterile PBS. In combination, mice were intraperitoneally (i.p.) injected with sterile PBS. Injections started once the tumor had reached a volume of 60-130 mm³ (average size of 92 mm³+/−19). The tumor volume of individual animals (n=15) is indicated (I) as mm³. The day of the first treatment is defined as day 0. Tumor volume was measured over the following days (II: days) with calipers. i.t. treatments (III) were performed on d0, d1, d5, d6, d10 and d11 and i.p. treatments (IV) were performed on d0, d4, d7, and d11.

FIG. 11: Tumor progression in wildtype Balb/c mice allografted s.c. with EMT-6 breast cancer cells treated with Y. enterocolitica ΔHOPEMT encoding YopE₁₋₁₃₈-murine RIG-I CARD₂. Wildtype Balb/c mice allografted s.c. with EMT-6 breast cancer cells were intratumorally (i.t.) injected with 7.5×10⁷ CFU of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-RIG-I CARD₂ is encoded under control of the yopE promoter). In combination, mice were intraperitoneally (i.p.) injected with sterile PBS. Injections started once the tumor had reached a volume of 60-130 mm³ (average size of 92 mm³+/−19). The tumor volume of individual animals (n=15) is indicated (I) as mm³. The day of the first treatment is defined as day 0. Tumor volume was measured over the following days (II: days) with calipers. i.t. treatments (III) were performed on d0, d1, d5, d6, d10 and d11 and i.p. treatments (IV) were performed on d0, d4, d7, and d11.

FIG. 12: Tumor progression in wildtype Balb/c mice allografted s.c. with EMT-6 breast cancer cells treated with an anti-PD-1 antibody. Wildtype Balb/c mice allografted s.c. with EMT-6 breast cancer cells were intratumorally (i.t.) injected with sterile PBS. In combination, mice were intraperitoneally (i.p.) injected with anti-PD-1 antibody (10 mg/kg per injection). Injections started once the tumor had reached a volume of 60-130 mm³ (average size of 92 mm³+/−19). The tumor volume of individual animals (n=15) is indicated (I) as mm³. The day of the first treatment is defined as day 0. Tumor volume was measured over the following days (II: days) with calipers. i.t. treatments (III) were performed on d0, d1, d5, d6, d10 and d11 and i.p. treatments (IV) were performed on d0, d4, d7, and d11.

FIG. 13: Tumor progression in wildtype Balb/c mice allografted s.c. with EMT-6 breast cancer cells treated with a combination of Y. enterocolitica ΔHOPEMT encoding YopE₁₋₁₃₈-murine RIG-I CARD₂ and an anti-PD-1 antibody. Wildtype Balb/c mice allografted s.c. with EMT-6 breast cancer cells were intratumorally (i.t.) injected with 7.5×10⁷ CFU of Y. enterocolitica ΔHOPEMT encoding YopE₁₋₁₃₈-murine RIG-I CARD₂ (RIG-11-246) on a medium copy number vector (whereon YopE₁₋₁₃₈-RIG-I CARD₂ is encoded under control of the yopE promoter). In combination, mice were intraperitoneally (i.p.) injected with anti-PD-1 antibody (10 mg/kg per injection). Injections started once the tumor had reached a volume of 60-130 mm³ (average size of 92 mm³+/−19). The tumor volume of individual animals (n=15) is indicated (I) as mm³. The day of the first treatment is defined as day 0. Tumor volume was measured over the following days (II: days) with calipers. i.t. treatments (III) were performed on d0, d1, d5, d6, d10 and d11 and i.p. treatments (IV) were performed on d0, d4, d7, and d11.

FIG. 14: Probability of survival of wildtype Balb/c mice allografted s.c. with EMT-6 breast cancer cells treated with either Y. enterocolitica ΔHOPEMT encoding YopE₁₋₁₃₈-murine RIG-I CARD₂, or with an anti-PD-1 antibody, or with a combination of both treatments. Wildtype Balb/c mice allografted s.c. with EMT-6 breast cancer cells were intratumorally (i.t.) injected with sterile PBS or with 7.5×10⁷ CFU of Y. enterocolitica ΔHOPEMT encoding YopE₁₋₁₃₈-murine RIG-I CARD₂ (RIG-11-246) on a medium copy number vector (whereon YopE₁₋₁₃₈-RIG-I CARD₂ is encoded under control of the yopE promoter). In combination, mice were intraperitoneally (i.p.) injected with anti-PD-1 antibody (10 mg/kg per injection) or with sterile PBS as a control. Injections started once the tumor had reached a volume of 60-130 mm³ (average size of 92 mm³+/−19).

The probability of survival (I, %) for each group over days (II) is represented. The day of the first treatment is defined as day 0. i.t. treatments (III) were performed on d0, d1, d5, d6, d10 and d11 and i.p. treatments (IV) were performed on d0, d4, d7, and d11.

Groups were distributed as (shown as: i.t. treatment+i.p. treatment): V. PBS+PBS; VI. Y. enterocolitica ΔHOPEMT encoding YopE₁₋₁₃₈-murine RIG-I CARD₂+PBS; VII. PBS+anti-PD-1; VIII. Y. enterocolitica ΔHOPEMT encoding YopE₁₋₁₃₈-murine RIG-I CARD₂+anti-PD-1.

FIG. 15: Tumor progression in wildtype Balb/c mice, either naïve or that previously experienced complete or partial tumour regression, challenged/rechallenged by s.c. allografting with EMT-6 breast cancer cells. Wildtype Balb/c mice, either naïve or all surviving mice whose first EMT6 tumors were either non-detectable (0 mm³), or smaller than 25 mm were challenged/rechallenged by s.c. allografting with EMT-6 breast cancer cells on the contralateral flank. The mean tumor volume at the contralateral flank (rechallenge tumour) is indicated (I) as mm³. The day of the first treatment is defined as day 0, rechallenge occurred at day 66 (III). Tumor volume was measured over the following days (II: days) with calipers. Groups were distributed as (shown as: i.t. treatment+i.p. treatment): IV. Y. enterocolitica ΔHOPEMT encoding YopE₁₋₁₃₈-murine RIG-I CARD₂+PBS (N=4); V. PBS+anti-PD-1 (N=1); VI. Y. enterocolitica ΔHOPEMT encoding YopE₁₋₁₃₈-murine RIG-I CARD₂+anti-PD-1 (N=7); VII. Naïve mice (N=10).

FIG. 16: Tumor progression in wildtype C57BL/6J mice allografted s.c. with B16F10 melanoma cells treated with either Y. enterocolitica ΔHOPEMT encoding YopE₁₋₁₃₈-murine RIG-I CARD₂, or with an anti-PD-1 antibody, or with a combination of both treatments. Wildtype C57BL/6J mice allografted s.c. with B16F10 melanoma cells were intratumorally (i.t.) injected with sterile PBS, or with 7.5×10⁷ CFU of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-RIG-I CARD₂ is encoded under control of the yopE promoter). In combination, mice were intraperitoneally (i.p.) injected with anti-PD-1 antibody or with sterile PBS as control. Data are displayed until each group contained less than 50% of initial mice alive.

Injections started once the tumor had reached a volume of 30-120 mm³ (average size of 71 mm³+/−25). The mean tumor volume is indicated (I) as mm³. The day of the first treatment is defined as day 0. Tumor volume was measured over the following days (II: days) with calipers. i.t. treatments (III) were performed on d0, d1, d2, d3, d6 and d9 and i.p. treatments (IV) were performed on d0, d4, d7, and d11.

Groups were distributed as (shown as: i.t. treatment+i.p. treatment): V. PBS+PBS; VI. Y. enterocolitica ΔHOPEMT encoding YopE₁₋₁₃₈-murine RIG-I CARD₂+PBS; VII. PBS+anti-PD-1; VIII. Y. enterocolitica ΔHOPEMT encoding YopE₁₋₁₃₈-murine RIG-I CARD₂+anti-PD-1.

FIG. 17: Tumor progression in wildtype C57BL/6J mice allografted s.c. with B16F10 melanoma cells of the control group. Wildtype C57BL/6J mice allografted s.c. with B16F10 melanoma cells were intratumorally (i.t.) injected with sterile PBS. In combination, mice were intraperitoneally (i.p.) injected with sterile PBS. Injections started once the tumor had reached a volume of 30-120 mm³ (average size of 71 mm³+/−25). The tumor volume of individual animals (n=15) is indicated (I) as mm³. The day of the first treatment is defined as day 0. Tumor volume was measured over the following days (II: days) with calipers. i.t. treatments (III) were performed on d0, d1, d2, d3, d6 and d9 and i.p. treatments (IV) were performed on d0, d4, d7, and d11.

FIG. 18: Tumor progression in wildtype C57BL/6J mice allografted s.c. with B16F10 melanoma cells treated with Y. enterocolitica ΔHOPEMT encoding YopE₁₋₁₃₈-murine RIG-I CARD₂. Wildtype C57BL/6J mice allografted s.c. with B16F10 melanoma cells were intratumorally (i.t.) injected with 7.5×10⁷ CFU of Y. enterocolitica ΔHOPEMT encoding YopE₁₋₁₃₈-murine RIG-I CARD₂ (RIG-11-246) on a medium copy number vector (whereon YopE₁₋₁₃₈-RIG-I CARD₂ is encoded under control of the yopE promoter). In combination, mice were intraperitoneally (i.p.) injected with sterile PBS. Injections started once the tumor had reached a volume of 30-120 mm³ (average size of 71 mm³+/−25). The tumor volume of individual animals (n=15) is indicated (I) as mm³. The day of the first treatment is defined as day 0. Tumor volume was measured over the following days (II: days) with calipers. i.t. treatments (III) were performed on d0, d1, d2, d3, d6 and d9 and i.p. treatments (IV) were performed on d0, d4, d7, and d11.

FIG. 19: Tumor progression in wildtype C57BL/6J mice allografted s.c. with B16F10 melanoma cells treated with an anti-PD-1 antibody. Wildtype C57BL/6J mice allografted s.c. with B16F10 melanoma cells were intratumorally (i.t.) injected with sterile PBS. In combination, mice were intraperitoneally (i.p.) injected with anti-PD-1 antibody (10 mg/kg per injection). Injections started once the tumor had reached a volume of 30-120 mm³ (average size of 71 mm³+/−25). The tumor volume of individual animals (n=15) is indicated (I) as mm³. The day of the first treatment is defined as day 0. Tumor volume was measured over the following days (II: days) with calipers. i.t. treatments (III) were performed on d0, d1, d2, d3, d6 and d9 and i.p. treatments (IV) were performed on d0, d4, d7, and d11.

FIG. 20: Tumor progression in wildtype C57BL/6J mice allografted s.c. with B16F10 melanoma cells treated with a combination of Y. enterocolitica ΔHOPEMT encoding YopE₁₋₁₃₈-murine RIG-I CARD₂ and an anti-PD-1 antibody. Wildtype C57BL/6J mice allografted s.c. with B16F10 melanoma cells were intratumorally (i.t.) injected with 7.5×10⁷ CFU of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-RIG-I CARD₂ is encoded under control of the yopE promoter). In combination, mice were intraperitoneally (i.p.) injected with anti-PD-1 antibody (10 mg/kg per injection). Injections started once the tumor had reached a volume of 30-120 mm³ (average size of 71 mm³+/−25). The tumor volume of individual animals (n=15) is indicated (I) as mm³. The day of the first treatment is defined as day 0. Tumor volume was measured over the following days (II: days) with calipers. i.t. treatments (III) were performed on d0, d1, d2, d3, d6 and d9 and i.p. treatments (IV) were performed on d0, d4, d7, and d11.

FIG. 21: Probability of survival of wildtype C57BL/6J mice allografted s.c. with B16F10 melanoma cells treated with either Y. enterocolitica ΔHOPEMT encoding YopE₁₋₁₃₈-murine RIG-I CARD₂, or with an anti-PD-1 antibody, or with a combination of both treatments. Wildtype C57BL/6J mice allografted s.c. with B16F10 melanoma cells were intratumorally (i.t.) injected with sterile PBS, or with 7.5×10⁷ CFU of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-RIG-I CARD₂ is encoded under control of the yopE promoter). In combination, mice were intraperitoneally (i.p.) injected with anti-PD-1 antibody (10 mg/kg per injection) or with sterile PBS as a control. The probability of survival (I, %) for each group over days (II) is represented. The day of the first treatment is defined as day 0. i.t. treatments (III) were performed on d0, d1, d2, d3, d6 and d9 and i.p. treatments (IV) were performed on d0, d4, d7, and d11.

Groups were distributed as (shown as: i.t. treatment+i.p. treatment): V. PBS+PBS; VI. Y. enterocolitica ΔHOPEMT encoding YopE₁₋₁₃₈-murine RIG-I CARD₂+PBS; VII. PBS+anti-PD-1; VIII. Y. enterocolitica ΔHOPEMT encoding YopE₁₋₁₃₈-murine RIG-I CARD₂+anti-PD-1.

FIG. 22: Tumor progression in wildtype C57BL/6J mice allografted s.c. with B16F10 melanoma cells of the control group. Wildtype C57BL/6J mice allografted s.c. with B16F10 melanoma cells were intravenously (i.v.) injected with sterile PBS. In combination, mice were intraperitoneally (i.p.) injected with control IgG (10 mg/kg per injection).

Injections started once the tumor had reached a volume of 40-120 mm³ (average size of 66 mm³+/−22). The tumor volume of individual animals (n=15) is indicated (I) as mm³. The day of the first treatment is defined as day 0. Tumor volume was measured over the following days (II: days) with calipers. i.v. treatments (III) were performed on d0, d2, d4, d6, d9, d13, d16 and i.p. treatments (IV) were performed on d0, d4, d6 and d9. * 5 mice sacrificed on d10

FIG. 23: Tumor progression in wildtype C57BL/6J mice allografted s.c. with B16F10 melanoma cells treated with Y. enterocolitica ΔHOPEMT encoding YopE₁₋₁₃₈-murine RIG-I CARD₂. Wildtype C57BL/6J mice allografted s.c. with B16F10 melanoma cells were intravenously (i.v.) injected with 1×10⁷ CFU of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-RIG-I CARD₂ is encoded under control of the yopE promoter). In combination, mice were intraperitoneally (i.p.) injected with control IgG (10 mg/kg per injection).

Injections started once the tumor had reached a volume of 40-120 mm³ (average size of 66 mm³+/−22). The tumor volume of individual animals (n=15) is indicated (I) as mm³. The day of the first treatment is defined as day 0. Tumor volume was measured over the following days (II: days) with calipers. i.v. treatments (III) were performed on d0, d2, d4, d6, d9, d13, d16 and i.p. treatments (IV) were performed on d0, d4, d6 and d9. * 5 mice sacrificed on d10

FIG. 24: Tumor progression in wildtype C57BL/6J mice allografted s.c. with B16F10 melanoma cells treated with an anti-PD-1 antibody. Wildtype C57BL/6J mice allografted s.c. with B16F10 melanoma cells were intravenously (i.v.) injected with sterile PBS. In combination, mice were intraperitoneally (i.p.) injected with anti-PD-1 antibody. Injections started once the tumor had reached a volume of 40-120 mm³ (average size of 66 mm³+/−22). The tumor volume of individual animals (n=15) is indicated (I) as mm³. The day of the first treatment is defined as day 0. Tumor volume was measured over the following days (II: days) with calipers. i.v. treatments (III) were performed on d0, d2, d4, d6, d9, d13, d16 and i.p. treatments (IV) were performed on d0, d4, d6 and d9. * 5 mice sacrificed on d10

FIG. 25: Tumor progression in wildtype C57BL/6J mice allografted s.c. with B16F10 melanoma cells treated with a combination of Y. enterocolitica ΔHOPEMT encoding YopE₁₋₁₃₈-murine RIG-I CARD₂ and an anti-PD-1 antibody. Wildtype C57BL/6J mice allografted s.c. with B16F10 melanoma cells were intravenously (i.v.) injected with 1×10⁷ CFU of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-RIG-I CARD₂ is encoded under control of the yopE promoter). In combination, mice were intraperitoneally (i.p.) injected with anti-PD-1 antibody. Injections started once the tumor had reached a volume of 40-120 mm³ (average size of 66 mm³+/−22). The tumor volume of individual animals (n=15) is indicated (I) as mm³. The day of the first treatment is defined as day 0. Tumor volume was measured over the following days (II: days) with calipers. i.v. treatments (III) were performed on d0, d2, d4, d6, d9, d13, d16 and d20 and i.p. treatments (IV) were performed on d0, d4, d6 and d9.* 5 mice sacrificed on d10

FIG. 26: Probability of survival of wildtype C57BL/6J mice allografted s.c. with B16F10 melanoma cells treated with either Y. enterocolitica ΔHOPEMT encoding YopE₁₋₁₃₈-murine RIG-I CARD₂, or with an anti-PD-1 antibody, or with a combination of both treatments. Wildtype C57BL/6J mice allografted s.c. with B16F10 melanoma cells were intravenously (i.v.) injected with sterile PBS, or with 1×10⁷ CFU of Y. enterocolitica ΔHOPEMT encoding YopE₁₋₁₃₈-murine RIG-I CARD₂ (RIG-11-246) on a medium copy number vector (whereon YopE₁₋₁₃₈-RIG-I CARD₂ is encoded under control of the yopE promoter). In combination, mice were intraperitoneally (i.p.) injected with anti-PD-1 antibody or with control IgG (10 mg/kg per injection). The probability of survival (I, %) for each group over days (II) is represented. The day of the first treatment is defined as day 0. i.v. treatments (III) were performed on d0, d2, d4, d6, d9, d13, d16 and d20 and i.p. treatments (IV) were performed on d0, d4, d6 and d9.

Groups were distributed as (shown as: i.v. treatment+i.p. treatment): V. PBS+control IgG; VI. Y. enterocolitica ΔHOPEMT encoding YopE₁₋₁₃₈-murine RIG-I CARD₂+control IgG; VII. PBS+anti-PD-1; VIII. Y. enterocolitica ΔHOPEMT encoding YopE₁₋₁₃₈-murine RIG-I CARD₂+anti-PD-1. * 5 mice per group were sacrificed on d10 and thus not considered in these calculations.

FIG. 27: Delivery of type I IFN inducing protein encoded on a medium-copy number vector. Delivery of human RIG-I CARD₂ or murine RIG-I CARD₂ leads to induction of type I IFN signalling in B16F1 melanocytes. B16F1 IFN reporter cells were infected with Y. enterocolitica ΔHOPEMT, either a control strain not delivering a cargo (III), or encoding on a medium-copy number vector IV: YopE₁₋₁₃₈-human RIG-I CARD₂, V: YopE₁₋₁₃₈-murine RIG-I CARD₂. A titration of the bacteria added to the cells was performed for each strain, indicated in I as Multiplicity of Infection (MOI). IFN stimulation was assessed based on activity of secreted alkaline phosphatase (II: OD₆₅₀) which is under the control of the I-ISG54 promoter which is comprised of the IFN-inducible ISG54 promoter enhanced by a multimeric ISRE.

FIG. 28: Average tumor progression in wildtype Balb/C mice allografted s.c. with CT26 colon carcinoma cells treated with either Y. enterocolitica ΔHOPEMT encoding YopE₁₋₁₃₈-human RIG-I CARD₂ and YopE₁₋₁₃₈-human cGAS₁₆₁₋₅₂₂, or with an anti-PD-1 antibody, or with a combination of both treatments. Wildtype Balb/c mice allografted s.c. with CT26 colon cancer cells were intratumorally (i.t.) injected with sterile PBS, or with 7.5×10⁷ CFU of Y. enterocolitica ΔHOPEMT encoding YopE₁₋₁₃₈-human RIG-I CARD₂ and YopE₁₋₁₃₈-human cGAS₁₆₁₋₅₂₂ both on the pYV and on a medium copy number vector (whereon YopE₁₋₁₃₈-human RIG-I CARD₂ and YopE₁₋₁₃₈-human cGAS₁₆₁₋₅₂₂ are encoded as an operon under control of the yopE promoter). In combination, mice were intraperitoneally (i.p.) injected with anti-PD-1 (Rat IgG2a, 10 mg/kg per injection) and where appropriate with control IgG2a isotype and/or with control IgG2b isotype.

Injections started once the tumor had reached a volume of 30-120 mm³ (average size of 58 mm³+/−19). The mean tumor volume is indicated (I) as mm³. The day of the first treatment is defined as day 0. Tumor volume was measured over the following days (II: days) with calipers. i.t. treatments (III) were performed on d0, d1, d3, d6, d10 and d14, IgG2a i.p. treatments (IV) were performed on d0, d4, d8, and d12, IgG2b treatments (V) were performed on d0, d2, d4, d6, d8, d10, d12 and d14. Data are displayed until each group contained less than 60% of initial mice alive.

Groups were distributed as (shown as: i.t. treatment+i.p. treatment): VI. PBS+ control IgG2a isotype+control IgG2b isotype; VII. Y. enterocolitica ΔHOPEMT encoding YopE₁₋₁₃₈-human RIG-I CARD₂ and YopE₁₋₁₃₈-human cGAS₁₆₁₋₅₂₂+control IgG2b isotype; VIII. PBS+anti-PD-1; IX. Y. enterocolitica ΔHOPEMT encoding YopE₁₋₁₃₈-human RIG-I CARD₂ and YopE₁₋₁₃₈-human cGAS₁₆₁₋₅₂₂+anti-PD-1, 13 animals per group.

FIG. 29: Tumor progression in wildtype Balb/C mice allografted s.c. with CT26 colon carcinoma cells of the control group. Wildtype Balb/c mice allografted s.c. with CT26 colon cancer cells were intratumorally (i.t.) injected with sterile PBS. In combination, mice were intraperitoneally (i.p.) injected with IgG2a and IgG2b control isotypes (10 mg/kg per injection).

Injections started once the tumor had reached a volume of 30-120 mm³ (average size of 58 mm³+/−19). The tumor volume of individual animals (n=13) is indicated (I) as mm³. The day of the first treatment is defined as day 0. Tumor volume was measured over the following days (II: days) with calipers. i.t. treatments (III) were performed on d0, d1, d3, d6, d10 and d14, i.p. treatments of IgG2a (IV) were performed on d0, d4, d8, and d12, and i.p. treatments of IgG2b (V) were performed on d0, d2, d4, d6, d8, d10, d12 and d14.

FIG. 30: Tumor progression in wildtype Balb/C mice allografted s.c. with CT26 colon carcinoma cells of the group treated with Y. enterocolitica ΔHOPEMT encoding YopE₁₋₁₃₈-human RIG-I CARD₂ and YopE₁₋₁₃₈-human cGAS₁₆₁₋₅₂₂. Wildtype Balb/c mice allografted s.c. with CT26 colon cancer cells were intratumorally (i.t.) injected with 7.5×10⁷ CFU of Y. enterocolitica ΔHOPEMT encoding YopE₁₋₁₃₈-human RIG-I CARD₂ and YopE₁₋₁₃₈-human cGAS₁₆₁₋₅₂₂ both on the pYV and on a medium copy number vector (whereon YopE₁₋₁₃₈-human RIG-I CARD₂ and YopE₁₋₁₃₈-human cGAS₁₆₁₋₅₂₂ are encoded as an operon under control of the yopE promoter). In combination, mice were intraperitoneally (i.p.) injected with IgG2b control isotypes.

Injections started once the tumor had reached a volume of 30-120 mm³ (average size of 58 mm³+/−19). The tumor volume of individual animals (n=13) is indicated (I) as mm³. The day of the first treatment is defined as day 0. Tumor volume was measured over the following days (II: days) with calipers. i.t. treatments (III) were performed on d0, d1, d3, d6, d10 and d14, and i.p. treatments of IgG2b (IV) were performed on d0, d2, d4, d6, d8, d10, d12 and d14.

FIG. 31: Tumor progression in wildtype Balb/C mice allografted s.c. with CT26 colon carcinoma cells of the group treated with an anti-PD-1 antibody. Wildtype Balb/c mice allografted s.c. with CT26 colon cancer cells were intratumorally (i.t.) injected with sterile PBS. In combination, mice were intraperitoneally (i.p.) injected with anti-PD-1 (Rat IgG2a, 10 mg/kg per injection).

Injections started once the tumor had reached a volume of 30-120 mm³ (average size of 58 mm³+/−19). The tumor volume of individual animals (n=13) is indicated (I) as mm³. The day of the first treatment is defined as day 0. Tumor volume was measured over the following days (II: days) with calipers. i.t. treatments (III) were performed on d0, d1, d3, d6, d10 and d14, and i.p. treatments of IgG2a anti-PD-1 (IV) were performed on d0, d4, d8, and d12.

FIG. 32: Tumor progression in wildtype Balb/C mice allografted s.c. with CT26 colon carcinoma cells of the group treated with combination of Y. enterocolitica ΔHOPEMT encoding YopE₁₋₁₃₈-human RIG-I CARD₂ and YopE₁₋₁₃₈-human cGAS₁₆₁₋₅₂₂ and an anti-PD-1 antibody. Wildtype Balb/c mice allografted s.c. with CT26 colon cancer cells were intratumorally (i.t.) injected with 7.5×10⁷ CFU of Y. enterocolitica ΔHOPEMT encoding YopE₁₋₁₃₈-human RIG-I CARD₂ and YopE₁₋₁₃₈-human cGAS₁₆₁₋₅₂₂ both on the pYV and on a medium copy number vector (whereon YopE₁₋₁₃₈-human RIG-I CARD₂ and YopE₁₋₁₃₈-human cGAS₁₆₁₋₅₂₂ are encoded as an operon under control of the yopE promoter). In combination, mice were intraperitoneally (i.p.) injected with anti-PD-1 (Rat IgG2a, 10 mg/kg per injection).

Injections started once the tumor had reached a volume of 30-120 mm³ (average size of 58 mm³+/−19). The tumor volume of individual animals (n=13) is indicated (I) as mm³. The day of the first treatment is defined as day 0. Tumor volume was measured over the following days (II: days) with calipers. i.t. treatments (III) were performed on d0, d1, d3, d6, d10 and d14, and i.p. treatments of IgG2a anti-PD-1 (IV) were performed on d0, d4, d8, and d12.

FIG. 33: The optimal tumour growth inhibition effect in wildtype Balb/C mice allografted s.c. with CT26 colon carcinoma cells of groups treated with Y. enterocolitica ΔHOPEMT encoding YopE₁₋₁₃₈-human RIG-I CARD₂ and YopE₁₋₁₃₈-human cGAS₁₆₁₋₅₂₂ and/or an anti-PD-1 antibody. The tumor growth inhibition (T/C %, I) is defined as the ratio of the median tumour volumes of treated animals versus median tumour volumes control animals (injection of sterile PBS/control IgG isotypes). The optimal value is the minimal T/C % ratio reflecting the maximal tumor growth inhibition achieved. The number of mice alive (II) at each considered optimal day (III) is indicated. T/C % ratios were classified as follows: 60-100%: no anti-tumoral activity (IV), 30-60%: marginal anti-tumoral activity (V), 10-30%: moderate anti-tumoral activity (VI), 0-10%: marked anti-tumoral activity (VII).

Groups were distributed as (shown as: i.t. treatment+i.p. treatment): VIII. PBS+control IgG2a isotype+control IgG2b isotype; IX. Y. enterocolitica ΔHOPEMT encoding YopE1-138-human RIG-I CARD2 and YopE1-138-human cGAS₁₆₁₋₅₂₂+control IgG2b isotype; X. Y. enterocolitica ΔHOPEMT encoding YopE1-138-human RIG-I CARD2 and YopE1-138-human cGAS₁₆₁₋₅₂₂+anti-PD-1, XI. PBS+anti-PD-1.

FIG. 34: List of strains as used in this application.

### DETAILED DESCRIPTION OF THE INVENTION

As outlined above, the present invention provides pharmaceutical combinations comprising a recombinant Gram-negative bacterial strain and an immune checkpoint modulator (ICM), which are useful for the prevention, delay of progression, or treatment of cancer.

Thus, in a first aspect the present invention provides a pharmaceutical combination comprising:
a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM), wherein the ICM is ezabenlimab; and optionally
(c) one or more pharmaceutically acceptable diluents, excipients or carriers.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

Features, integers, characteristics, compounds described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments.

The term “comprise” and variations thereof, such as, “comprises” and “comprising” is generally used in the sense of include, that is, as “including, but not limited to”, that is to say permitting the presence of one or more features or components.

The singular forms “a,” “an,” and “the” include plural referents unless the content clearly dictates otherwise.

The term “about” refers to a range of values ±10% of a specified value. For example, the phrase “about 200” includes ±10% of 200, or from 180 to 220.

The term “Gram-negative bacterial strain” as used herein includes the following bacteria: Aeromonas salmonicida, Aeromonas hydrophila, Aeromonas veronii, Anaeromyxobacter dehalogenans, Bordetella bronchiseptica, Bordetella parapertussis, Bordetella pertussis, Bradyrhizobium japonicum, Burkholderia cenocepacia, Burkholderia cepacia, Burkholderia mallei, Burkholderia pseudomallei, Chlamydia muridarum, Chlamydia trachmoatis, Chlamydophila abortus, Chlamydophila pneumoniae, Chromobacterium violaceum, Citrobacter rodentium, Desulfovibrio vulgaris, Edwardsiella tarda, Endozoicomonas elysicola, Erwinia amylovora, Escherichia albertii, Escherichia coli, Lawsonia intracellularis, Mesorhizobium loti, Myxococcus xanthus, Pantoea agglomerans, Photobacterium damselae, Photorhabdus luminescens, Photorabdus temperate, Pseudoalteromonas spongiae, Pseudomonas aeruginosa, Pseudomonas plecoglossicida, Pseudomonas syringae, Ralstonia solanacearum, Rhizobium sp, Salmonella enterica and other Salmonella sp, Shigella flexneri and other Shigella sp, Sodalis glossinidius, Vibrio alginolyticus, Vibrio azureus, Vibrio campellii, Vibrio caribbenthicus, Vibrio harvey, Vibrio parahaemolyticus, Vibrio tasmaniensis, Vibrio tubiashii, Xanthomonas axonopodis, Xanthomonas campestris, Xanthomonas oryzae, Yersinia enterocolitica, Yersinia pestis, Yersinia pseudotuberculosis. Preferred Gram-negative bacterial strains of the invention are Gram-negative bacterial strains comprised by the family of Enterobacteriaceae and Pseudomonadaceae. The Gram-negative bacterial strain of the present invention is normally used for delivery of heterologous proteins by the bacterial T3SS into eukaryotic cells in vitro and/or in vivo, preferably in vivo.

The term “recombinant Gram-negative bacterial strain” as used herein refers to a recombinant Gram-negative bacterial strain genetically transformed with a polynucleotide construct like a vector. The recombinant Gram-negative bacterial strain of the present invention is genetically modified through transformation, transduction or conjugation, and is preferably a recombinant Gram-negative bacterial strain genetically modified through transformation, transduction or conjugation with a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter. Virulence of such a recombinant Gram-negative bacterial strain is usually attenuated by deletion of bacterial effector proteins having virulence activity which are transported by one or more bacterial proteins, which are part of a secretion system machinery. Such effector proteins are delivered by a secretion system machinery into a host cells where they exert their virulence activity toward various host proteins and cellular machineries. Many different effector proteins are known, transported by various secretion system types and displaying a large repertoire of biochemical activities that modulate the functions of host regulatory molecules. Virulence of the recombinant Gram-negative bacterial strain used herein can be attenuated additionally by lack of a siderophore normally or occasionally produced by the Gram-negative bacterial strain so that the strain does not produce the siderophore e.g. is deficient in the production of the siderophore. Thus in a preferred embodiment a recombinant Gram-negative bacterial strain is used which lacks of a siderophore normally or occasionally produced by the Gram-negative bacterial strain so that the strain does not produce the siderophore e.g. is deficient in the production of a siderophore, more preferably a Yersinia strain, in particular Y. enterocolitica MRS40 ΔyopH,O,P,E,M,T, Y. enterocolitica MRS40 ΔyopH,O,P,E,M,T ΔHairpinI-virF or Y. enterocolitica MRS40 ΔyopH,O,P,E,M,T Δasd pYV-asd is used which lack of a siderophore normally or occasionally produced by the Gram-negative bacterial strain so that the strain does not produce the siderophore e.g. is deficient in the production of a siderophore, in particular is deficient in the production of Yersiniabactin. Most preferably a Yersinia strain, in particular Y. enterocolitica MRS40 ΔyopH,O,P,E,M,T is used which lack of a siderophore normally or occasionally produced by the Gram-negative bacterial strain so that the strain does not produce the siderophore e.g. is deficient in the production of a siderophore, in particular is deficient in the production of Yersiniabactin. Y. enterocolitica MRS40 ΔyopH,O,P,E,M,T which is deficient in the production of Yersiniabactin has been described in WO02077249 and was deposited on 24 Sep. 2001, according to the Budapest Treaty on the International Recognition of the Deposit of Microorganisms for the Purposes of Patent Procedure with the Belgian Coordinated Collections of Microorganisms (BCCM) and was given accession number LMG P-21013. The recombinant Gram-negative bacterial strain preferably does not produce a siderophore e.g. is deficient in the production of a siderophore.

The term “siderophore”, “iron siderophore” or “iron chelator” which are used interchangeably herein refer to compounds with high affinity for iron e.g. small compounds with high affinity for iron.

Siderophores of Gram-negative bacteria are e.g. Enterobactin and dihydroxybenzoylserine synthetized by Salmonella, Escherichia, Klebsiella, Shigella, Serratia (but used by all enterobacteria), Pyoverdins synthetized by Pseudomonas, Vibriobactin synthetized by Vibrio, Acinetobactin and Acinetoferrin by Acinetobacter, Yersiniabactin and Aerobactin synthetized by Yersinia, Ornibactin synthetized by Burkholderia, Salmochelin synthetized by Salmonella, Aerobactin synthetized by Escherichia, Shigella, Salmonella, and Yersinia, Alcaligin synthetized by Bordetella, Bisucaberin synthetized by Vibrio.

Siderophores include hydroxamate, catecholate and mixed ligand siderophores. Several siderophores have to date been approved for use in humans, mainly with the aim of treating iron overload. Preferred siderophores are Deferoxamine (also known as desferrioxamine B, desferoxamine B, DFO-B, DFOA, DFB or desferal), Desferrioxamine E, Deferasirox (Exjade, Desirox, Defrijet, Desifer) and Deferiprone (Ferriprox).

The term “an endogenous protein essential for growth” used herein refers to proteins of the recombinant Gram-negative bacterial strain without those the Gram-negative bacterial strain cannot grow. Endogenous proteins essential for growth are e.g. an enzyme essential for amino acid production, an enzyme involved in peptidoglycan biosynthesis, an enzyme involved in LPS biosynthesis, an enzyme involved in nucleotide synthesis or a translation initiation factor.

The term “an enzyme essential for amino acid production” used herein refers to enzymes which are related to the amino acid production of the recombinant Gram-negative bacterial strain and without those the Gram-negative bacterial strain can not grow. Enzymes essential for amino acid production, are e.g. aspartate-beta-semialdehyde dehydrogenase (asd), glutamine synthetase (glnA), tryptophanyl tRNA synthetase (trpS) or serine hydroxymethyl transferase (glyA), or Transketolase 1 (tktA), Transketolase 2 (tktB), Ribulose-phosphate 3-epimerase (rpe), Ribose-5-phosphate isomerase A (rpiA), Transaldolase A (talA), Transaldolase B (talB), phosphoribosylpyrophosphate synthase (prs), ATP phosphoribosyltransferase (hisG), Histidine biosynthesis bifunctional protein HisIE (hisI), 1-(5-phosphoribosyl)-5-[(5-phosphoribosylamino)methylideneamino] imidazole-4-carboxamide isomerase (hisA), Imidazole glycerol phosphate synthase subunit HisH (hisH), Imidazole glycerol phosphate synthase subunit HisF (hisF), Histidine biosynthesis bifunctional protein HisB (hisB), Histidinol-phosphate aminotransferase (hisC), Histidinol dehydrogenase (hisD), 3-dehydroquinate synthase (aroB), 3-dehydroquinate dehydratase (aroD), Shikimate dehydrogenase (NADP(+)) (aroE), Shikimate kinase 2 (aroL), Shikimate kinase 1 (aroK), 3-phosphoshikimate 1-carboxyvinyltransferase (aroA), Chorismate synthase (aroC), P-protein (pheA), T-protein (tyrA), Aromatic-amino-acid aminotransferase (tyrB), Phospho-2-dehydro-3-deoxyheptonate aldolase (aroG), Phospho-2-dehydro-3-deoxyheptonate aldolase (aroH), Phospho-2-dehydro-3-deoxyheptonate aldolase (aroF), Quinate/shikimate dehydrogenase (ydiB), ATP-dependent 6-phosphofructokinase isozyme 1 (pfkA), ATP-dependent 6-phosphofructokinase isozyme 2 (pfkB), Fructose-bisphosphate aldolase class 2 (fbaA), Fructose-bisphosphate aldolase class 1 (fbaB), Triosephosphate isomerase (tpiA), Pyruvate kinase I (pykF), Pyruvate kinase II (pykA), Glyceraldehyde-3-phosphate dehydrogenase A (gapA), Phosphoglycerate kinase (pgk), 2,3-bisphosphoglycerate-dependent phosphoglycerate mutase (gpmA), 2,3-bisphosphoglycerate-independent phosphoglycerate mutase (gpmM/yibO), Probable phosphoglycerate mutase (ytjC/gpmB), enolase (eno), D-3-phosphoglycerate dehydrogenase (serA), Phosphoserine aminotransferase (serC), Phosphoserine phosphatase (serB), L-serine dehydratase 1 (sdaA), L-serine dehydratase 2 (sdaB), L-threonine dehydratase catabolic (tdcB), L-threonine dehydratase biosynthetic (ilvA), L-serine dehydratase (tdcG), Serine acetyltransferase (cysE), Cysteine synthase A (cysK), Cysteine synthase B (cysM), beta-cystathionase (malY), Cystathionine beta-lyase (metC), 5-methyltetrahydropteroyltriglutamate-homocysteine methyltransferase (metE), Methionine synthase (metH), S-adenosylmethionine synthase (metK), Cystathionine gamma-synthase (metB), Homoserine O-succinyltransferase (metA), 5′-methylthioadenosine/S-adenosylhomocysteine nucleosidase (mtnN), S-ribosylhomocysteine lyase (luxS), cystathione beta lyase, cystathione gamma lyase, Serine hydroxymethyltransferase (glyA), Glycine hydroxymethyltransferase (itaE), 3-isopropylmalate dehydratase small subunit (leuD), 3-isopropylmalate dehydratase large subunit (leuC), 3-isopropylmalate dehydrogenase (leuB), L-threonine dehydratase biosynthetic (ilvA), Acetolactate synthase isozyme 3 large subunit (ilvI), Acetolactate synthase isozyme 3 small subunit (ilvH), Acetolactate synthase isozyme 1 small subunit (ilvN), Acetolactate synthase isozyme 2 small subunit (ilvM), Ketol-acid reductoisomerase (NADP(+)) (ilvC), Dihydroxy-acid dehydratase (ilvD), Branched-chain-amino-acid aminotransferase (ilvE), Bifunctional aspartokinase/homoserine dehydrogenase 1 (thrA), Bifunctional aspartokinase/homoserine dehydrogenase 2 (metL), 2-isopropylmalate synthase (leuA), Glutamate-pyruvate aminotransferase (alaA), Aspartate aminotransferase (aspC), Bifunctional aspartokinase/homoserine dehydrogenase 1 (thrA), Bifunctional aspartokinase/homoserine dehydrogenase 2 (metL), Lysine-sensitive aspartokinase 3 (lysC), Aspartate-semialdehyde dehydrogenase (asd), 2-keto-3-deoxy-galactonate aldolase (yagE), 4-hydroxy-tetrahydrodipicolinate synthase (dapA), 4-hydroxy-tetrahydrodipicolinate reductase (dapB), 2,3,4,5-tetrahydropyridine-2,6-dicarboxylate N-succinyltransferase (dapD), Succinyldiaminopimelate desuccinylase (dapE), Diaminopimelate epimerase (dapF), Putative lyase (yjhH), Acetylornithine/succinyldiaminopimelate aminotransferase (argD), Citrate synthase (gltA), Aconitate hydratase B (acnB), Aconitate hydratase A (acnA), uncharacterized putative aconitate hydratase (ybhJ), isocitrate dehydrogenase (icd), Aspartate aminotransferase (aspC), Glutamate-pyruvate aminotransferase (alaA), Glutamate synthase [NADPH] large chain (gltB), Glutamate synthase [NADPH] small chain (gltD), Glutamine synthetase (glnA), Amino-acid acetyltransferase (argA), Acetylglutamate kinase (argB), N-acetyl-gamma-glutamyl-phosphate reductase (argC), Acetylornithine/succinyldiaminopimelate aminotransferase (argD), Acetylornithine deacetylase (argE), Ornithine carbamoyltransferase chain F (argF), Ornithine carbamoyltransferase chain I (argI), Argininosuccinate synthase (argG), Argininosuccinate lyase (argH), Glutamate 5-kinase (proB), Gamma-glutamyl phosphate reductase (proA), pyrroline-5-carboxylate reductase (proC), ornithine cyclodeaminase, Leucine-tRNA ligase (leuS), Glutamine-tRNA ligase (glnS), Serine-tRNA ligase (serS), Glycine-tRNA ligase beta subunit (glyS), Glycine-tRNA ligase alpha subunit (glyQ), Tyrosine-tRNA ligase (tyrS), Threonine-tRNA ligase (thrS), Phenylalanine-tRNA ligase alpha subunit (pheS), Phenylalanine-tRNA ligase beta subunit (pheT), Arginine-tRNA ligase (argS), Histidine-tRNA ligase (hisS), Valine-tRNA ligase (valS), Alanine-tRNA ligase (alaS), Isoleucine-tRNA ligase (ileS), Proline-tRNA ligase (proS), Cystein-tRNA ligase (cysS), Asparagine-tRNA ligase (asnS), Aspartate-tRNA ligase (aspS), Glutamate-tRNA ligase (gltX), Tryptophan-tRNA ligase (trpS), Glycine-tRNA ligase beta subunit (glyS), Methionine-tRNA ligase (metG), Lysine-tRNA ligase (lysS). Preferred enzymes essential for amino acid production are tktA, rpe, prs, aroK, tyrB, aroH, fbaA, gapA, pgk, eno, tdcG, cysE, metK, glyA, asd, dapA/B/D/E/F, argC, proC, leuS, glnS, serS, glyS/Q, tyrS, thrS, pheS/T, argS, hisS, valS, alaS, ileS, proS, cysS, asnS, aspS, gltX, trpS, glyS, metG, lysS, more preferred are asd, glyA, leuS, glnS, serS, glyS/Q, tyrS, thrS, pheS/T, argS, hisS, valS, alaS, ileS, proS, cysS, asnS, aspS, gltX, trpS, glyS, metG, lysS, most preferred is asd.

The terms “Gram-negative bacterial strain deficient to produce an amino acid essential for growth” and “auxotroph mutant” are used herein interchangeably and refer to Gram-negative bacterial strains which can not grow in the absence of at least one exogenously provided essential amino acid or a precursor thereof. The amino acid the strain is deficient to produce is e.g. aspartate, meso-2,6-diaminopimelic acid, aromatic amino acids or leucine-arginine. Such a strain can be generated by e.g. deletion of the aspartate-beta-semialdehyde dehydrogenase gene (Δasd). Such an auxotroph mutant cannot grow in absence of exogenous meso-2,6-diaminopimelic acid. The mutation, e.g. deletion of the aspartate-beta-semialdehyde dehydrogenase gene is preferred herein for a Gram-negative bacterial strain deficient to produce an amino acid essential for growth of the present invention.

The term “Gram-negative bacterial strain deficient to produce adhesion proteins binding to the eukaryotic cell surface or extracellular matrix” refers to mutant Gram-negative bacterial strains which do not express at least one adhesion protein compared to the adhesion proteins expressed by the corresponding wild type strain. Adhesion proteins may include e.g. extended polymeric adhesion molecules like pili/fimbriae or non-fimbrial adhesins. Fimbrial adhesins include type-1 pili (such as E. coli Fim-pili with the FimH adhesin), P-pili (such as Pap-pili with the PapG adhesin from E. coli), type 4 pili (as pilin protein from e.g. P. aeruginosa) or curli (Csg proteins with the CsgA adhesin from S. enterica). Non-fimbrial adhesions include trimeric autotransporter adhesins such as YadA from Y. enterocolitica, BpaA (B. pseudomallei), Hia (H. influenzae), BadA (B. henselae), NadA (N. meningitidis) or UspA1 (M. catarrhalis) as well as other autotransporter adhesins such as AIDA-1 (E. coli) as well as other adhesins/invasins such as InvA from Y. enterocolitica or Intimin (E. coli) or members of the Dr-family or Afa-family (E. coli). The terms YadA and InvA as used herein refer to proteins from Y. enterocolitica. The autotransporter YadA (Skurnik and Wolf-Watz, 1989) binds to different froms of collagen as well as fibronectin, while the invasin InvA (Isberg et al., 1987) binds to β-integrins in the eukaryotic cell membrane. If the Gram-negative bacterial strain is a Y. enterocolitica strain the strain is preferably deficient in InvA and/or YadA.

As used herein, the term “family of Enterobacteriaceae” comprises a family of gram-negative, rod-shaped, facultatively anaerobic bacteria found in soil, water, plants, and animals, which frequently occur as pathogens in vertebrates. The bacteria of this family share a similar physiology and demonstrate a conservation within functional elements and genes of the respective genomes. As well as being oxidase negative, all members of this family are glucose fermenters and most are nitrate reducers.

Enterobacteriaceae bacteria of the invention may be any bacteria from that family, and specifically includes, but is not limited to, bacteria of the following genera: Escherichia, Shigella, Edwardsiella, Salmonella, Citrobacter, Klebsiella, Enterobacter, Serratia, Proteus, Erwinia, Morganella, Providencia, or Yersinia. In more specific embodiments, the bacterium is of the Escherichia coli, Escherichia blattae, Escherichia fergusonii, Escherichia hermanii, Escherichia vuneris, Salmonella enterica, Salmonella bongori, Shigella dysenteriae, Shigella flexneri, Shigella boydii, Shigella sonnei, Enterobacter aerogenes, Enterobacter gergoviae, Enterobacter sakazakii, Enterobacter cloacae, Enterobacter agglomerans, Klebsiella pneumoniae, Klebsiella oxytoca, Serratia marcescens, Yersinia pseudotuberculosis, Yersinia pestis, Yersinia enterocolitica, Erwinia amylovora, Proteus mirabilis, Proteus vulgaris, Proteus penneri, Proteus hauseri, Providencia alcalifaciens, or Morganella morganii species. Preferably the Gram-negative bacterial strain is selected from the group consisting of the genera Yersinia, Escherichia, Salmonella, Shigella, Pseudomonas, Chlamydia, Erwinia, Pantoea, Vibrio, Burkholderia, Ralstonia, Xanthomonas, Chromobacterium, Sodalis, Citrobacter, Edwardsiella, Rhizobiae, Aeromonas, Photorhabdus, Bordetella and Desulfovibrio, more preferably from the group consisting of the genera Yersinia, Escherichia, Salmonella, and Pseudomonas, most preferably from the group consisting of the genera Yersinia and Salmonella, in particular Yersinia.

The term “Yersinia” as used herein includes all species of Yersinia, including Yersinia enterocolitica, Yersinia pseudotuberculosis and Yersinia pestis. Preferred is Yersinia enterocolitica.

The term “Salmonella” as used herein includes all species of Salmonella, including Salmonella enterica and S. bongori. Preferred is Salmonella enterica.

“Promoter” as used herein refers to a nucleic acid sequence that regulates expression of a transcriptional unit. A “promoter region” is a regulatory region capable of binding RNA polymerase in a cell and initiating transcription of a downstream (3′ direction) coding sequence. Within the promoter region will be found a transcription initiation site (conveniently defined by mapping with nuclease S1), as well as protein binding domains (consensus sequences) responsible for the binding of RNA polymerase such as the putative-35 region and the Pribnow box. The term “operably linked” when describing the relationship between two nucleotide e.g. DNA regions simply means that they are functionally related to each other and they are located on the same nucleic acid fragment. A promoter is operably linked to a structural gene if it controls the transcription of the gene and it is located on the same nucleic acid fragment as the gene. Usually the promoter is functional in said Gram-negative bacterial strain, i.e. the promoter is capable of expressing the fusion protein of the present invention, i.e. the promoter is capable of expressing the fusion protein of the present invention without further genetic engineering or expression of further proteins. Furthermore, a functional promoter must not be naturally counter-regulated to the bacterial T3SS.

The term “extra-chromosomal genetic element” used herein refers to a genetic element other than a chromosome which is endogenously harboured by the Gram-negative bacterial strain of the present invention such as a virulence plasmid or which is an exogenous genetic element with which the Gram-negative bacterial strain is transformed and which is transiently or stably integrated into the chromosome or into a genetic element other than a chromosome which is endogenously harboured such as an endogenous virulence plasmid. An endogenous virulence plasmid is the preferred extra-chromosomal genetic element of the present invention. Such an extra-chromosomal genetic element may be generated by integration of a vector like an expression vector, a vector for homologous recombination or other integration into the chromosome or into a genetic element other than a chromosome which is endogenously harboured such as a virulence plasmid, by integration of DNA fragments for homologous recombination or other integration into the chromosome or into a genetic element other than a chromosome which is endogenously harboured such as a virulence plasmid or via an RNA element guiding site specific insertion into the chromosome or into a genetic element other than a chromosome which is endogenously harboured such as a virulence plasmid, such as CRISPR/Cas9 and related guide RNA.

The terms “polynucleic acid molecule” and “polynucleotide molecule” are used herein interchangeably and have the identical meaning herein, and refer to both DNA and RNA molecules, which can either be single-stranded or double-stranded, and that can be partially or fully transcribed and translated (DNA), or partially or fully translated (RNA), into a gene product.

The terms “nucleic acid sequence”, “nucleotide sequence” and “nucleotide acid sequence” are used herein interchangeably and have the identical meaning herein, and refer to preferably DNA or RNA. The terms “nucleic acid sequence”, “nucleotide sequence” and “nucleotide acid sequence” are preferably used synonymous with the term “polynucleotide sequence”.

The term “operon” used herein refers to two or more genes transcribed under the control of a single promoter. These genes are thus typically transcribed together and form one messenger RNA, whereat this single mRNA encodes more than one protein (polycistronic mRNA).

Additionally to a promoter and two or more genes, an operator element may also be present, which controls transcription.

The term “delivery” used herein refers to the transportation of a protein from a recombinant Gram-negative bacterial strain to a eukaryotic cell, including the steps of expressing the heterologous protein in the recombinant Gram-negative bacterial strain, secreting the expressed protein(s) from such recombinant Gram-negative bacterial strain and translocating the secreted protein(s) by such recombinant Gram-negative bacterial strain into the cytosol of the eukaryotic cell. Accordingly, the terms “delivery signal” or “secretion signal” which are used interchangeably herein refer to a polypeptide sequence which can be recognized by the secretion and translocation system of the Gram-negative bacterial strain and directs the delivery of a protein from the Gram-negative bacterial strain to eukaryotic cells.

The term “delivery signal from a bacterial effector protein” used herein refers to a delivery signal from a bacterial effector protein functional in the recombinant Gram-negative bacterial strain, i.e. which allows an expressed heterologous protein in the recombinant Gram-negative bacterial strain to be secreted from such recombinant Gram-negative bacterial strain by a secretion system such as the type III, type IV or type VI secretion system or to be translocated by such recombinant Gram-negative bacterial strain into the cytosol of a eukaryotic cell by a secretion system such as the type III, type IV or type VI secretion system. The term “delivery signal from a bacterial effector protein” used herein also comprises a fragment of a delivery signal from a bacterial effector protein i.e. shorter versions of a delivery signal e.g. a delivery signal comprising up to 10, preferably up to 20, more preferably up to 50, even more preferably up to 100, in particular up to 140 amino acids of a delivery signal e.g. of a naturally occurring delivery signal. Thus a nucleotide sequence such as e.g. a DNA sequence encoding a delivery signal from a bacterial effector protein may encode a full length delivery signal or a fragment thereof wherein the fragment usually comprises usually up to 30, preferably up to 60, more preferably up to 150, even more preferably up to 300, in particular up to 420 nucleic acids.

As used herein, the “secretion” of a protein refers to the transportation of a heterologous protein outward across the cell membrane of a recombinant Gram-negative bacterial strain. The “translocation” of a protein refers to the transportation of a heterologous protein from a recombinant Gram-negative bacterial strain across the plasma membrane of a eukaryotic cell into the cytosol of such eukaryotic cell.

The term “bacterial protein, which is part of a secretion system machinery” as used herein refers to bacterial proteins constituting essential components of the bacterial type 3 secretion system (T3SS), type 4 secretion system (T4SS) and type 6 secretion system (T6SS), preferably T3SS. Without such proteins, the respective secretion system is non-functional in translocating proteins to host cells, even if all other components of the secretion system and the bacterial effector protein to be translocated are still encoded and produced.

The term “bacterial effector protein” as used herein refers to bacterial proteins transported by secretion systems e.g. by bacterial proteins, which are part of a secretion system machinery into host cells. Such effector proteins are delivered by a secretion system into a host cell where they exert e.g. virulence activity toward various host proteins and cellular machineries. Many different effector proteins are known, transported by various secretion system types and displaying a large repertoire of biochemical activities that modulate the functions of host regulatory molecules. Secretion systems include type 3 secretion system (T3SS), type 4 secretion system (T4SS) and type 6 secretion system (T6SS). Some effector proteins (as Shigella flexneri IpaC) as well belong to the class of bacterial protein, which are part of a secretion system machinery and allow protein translocation. The recombinant Gram-negative bacterial strain used herein usually comprises bacterial proteins constituting essential components of the bacterial type 3 secretion system (T3SS), type 4 secretion system (T4SS) and/or the type 6 secretion system (T6SS), preferably of the type 3 secretion system (T3SS). Bacterial effector proteins of the recombinant Gram-negative bacterial strain of the present invention are usually bacterial T3SS effector proteins, bacterial T4SS effector proteins or bacterial T6SS effector proteins, preferably bacterial T3SS effector proteins.

The term “bacterial proteins constituting essential components of the bacterial T3SS” as used herein refers to proteins, which are naturally forming the injectisome e.g. the injection needle or are otherwise essential for its function in translocating proteins into eukaryotic cells. Proteins forming the injectisome or are otherwise essential for its function in translocating proteins into eukaryotic cells include, but are not limited to:

SctC, YscC, MxiD, InvG, SsaC, EscC, HrcC, HrcC (Secretin), SctD, YscD, MxiG, Prg, SsaD, EscD, HrpQ, HrpW, FliG (Outer MS ring protein), SctJ, YscJ, MxiJ, PrgK, SsaJ, EscJ, HrcJ, HrcJ, FliF (Inner MS ring protein), SctR, YscR, Spa24, SpaP, SpaP, SsaR, EscR, HrcR, HrcR, FliP (Minor export apparatus protein), SctS, YscS, Spa9 (SpaQ), SpaQ, SsaS, EscS, HrcS, HrcS, FliQ (Minor export apparatus protein), SctT, YscT, Spa29 (SpaR), SpaR, SsaT, EscT, HrcT, HrcT, FliR (Minor export apparatus protein), SctU, YscU, Spa40, SpaS, SpaS, SsaU, EscU, HrcU, HrcU, FlhB (Export apparatus switch protein), SctV, YscV, MxiA, InvA, SsaV, EscV, HrcV, HrcV, FlhA (Major export apparatus protein), SctK, YscK, MxiK, OrgA, HrpD (Accessory cytosolic protein), SctQ, YscQ, Spa33, SpaO, SpaO, SsaQ, EscQ, HrcQA+B, HrcQ, FliM+FliN (C ring protein), SctL, YscL, MxiN, OrgB, Ssak, EscL, Orf5, HrpE, HrpF, FliH (Stator), SctN, YscN, Spa47, SpaL, InvC, SsaN, EscN, HrcN, HrcN, FliI (ATPase), SctO, YscO, Spa13, SpaM, InvI, SsaO, Orf15, HrpO, HrpD, FliJ (Stalk), SctF, YscF, MxiH, PrgI, SsaG, EscF, HrpA, HrpY (Needle filament protein), SctI, YscI, MxiI, PrgJ, SsaI, EscI, rOrf8, HrpB, HrpJ, (Inner rod protein), SctP, YscP, Spa32, SpaN, InvJ, SsaP, EscP, Orf16, HrpP, HpaP, FliK (Needle length regulator), LcrV, IpaD, SipD (Hydrophilic translocator, needle tip protein), YopB, IpaB, SipB, SseC, EspD, HrpK, PopF1, PopF2 (Hydrophobic translocator, pore protein), YopD, IpaC, SipC, SseD, EspB (Hydrophobic translocator, pore protein), YscW, MxiM, InvH (Pilotin), SctW, YopN, MxiC, InvE, SsaL, SepL, HrpJ, HpaA (Gatekeeper).

The term “T6SS effector protein” or “bacterial T6SS effector protein” as used herein refers to proteins which are naturally injected by T6S systems into the cytosol of eukaryotic cells or bacteria and to proteins which are naturally secreted by T6S systems that might e.g. form translocation pores into the eukaryotic membrane. The term “T4SS effector protein” or “bacterial T4SS effector protein” as used herein refers to proteins which are naturally injected by T4S systems into the cytosol of eukaryotic cells and to proteins which are naturally secreted by T4S systems that might e.g. form the translocation pore into the eukaryotic membrane. The term “T3SS effector protein” or “bacterial T3SS effector protein” as used herein refers to proteins which are naturally injected by T3S systems into the cytosol of eukaryotic cells and to proteins which are naturally secreted by T3S systems that might e.g. form the translocation pore into the eukaryotic membrane (including pore-forming translocators (as Yersinia YopB and YopD) and tip-proteins like Yersinia LcrV). Preferably proteins which are naturally injected by T3S systems into the cytosol of eukaryotic cells are used. These virulence factors will paralyze or reprogram the eukaryotic cell to the benefit of the pathogen. T3S effectors display a large repertoire of biochemical activities and modulate the function of crucial host regulatory molecules and include, but are not limited to, AvrA, AvrB, AvrBs2, AvrBS3, AvrBsT, AvrD, AvrD1, AvrPphB, AvrPphC, AvrPphEPto, AvrPpiBPto, AvrPto, AvrPtoB, AvrRpm1, AvrRpt2, AvrXv3, CigR, EspF, EspG, EspH, EspZ, ExoS, ExoT, GogB, GtgA, GtgE, GALA family of proteins, HopAB2, HopAO1, HopI1, HopM1, HopN1, HopPtoD2, HopPtoE, HopPtoF, HopPtoN, HopU1, HsvB, IcsB, IpaA, IpaB, IpaC, IpaH, IpaH7.8, IpaH9.8, IpgB1, IpgB2, IpgD, LcrV, Map, OspC1, OspE2, OspF, OspG, OspI, PipB, PipB2, PopB, PopP2, PthXo1, PthXo06, PthXo7, SifA, SifB, SipA/SspA, SipB, SipC/SspC, SipD/SspD, SlrP, SopA, SopB/SigD, SopD, SopE, SopE2, SpiC/SsaB, SptP, SpvB, SpvC, SrfH, SrfJ, Sse, SseB, SseC, SseD, SseF, SseG, SseI/SrfH, SseJ, SseK1, SseK2, SseK3, SseL, SspH1, SspH2, SteA, SteB, SteC, SteD, SteE, TccP2, Tir, VirA, VirPphA, VopF, XopD, YopB, YopD YopE, YopH, YopJ, YopM, YopO, YopP, YopT, YpkA.

The term “recombinant Gram-negative bacterial strain accumulating in a malignant solid tumor” or “the recombinant Gram-negative bacterial strain accumulates in a malignant solid tumor” as used herein refers to a recombinant Gram-negative bacterial strain which replicates within a malignant solid tumor thereby increasing the bacterial count of this recombinant Gram-negative bacterial strain inside the malignant solid tumor. Surprisingly it has been found that the recombinant Gram-negative bacterial strain after administration to the subject accumulates specifically in the malignant solid tumor i.e. accumulates specifically in the organ where the malignant tumor is present, wherein the bacterial counts of the recombinant Gram-negative bacterial strain in organs where no malignant solid tumor is present is low or not detectable. In case of extracellular residing bacteria as Yersinia, the bacteria mostly accumulate within the intercellular space formed between tumor cells or cells of the tumor microenvironment. Intracellular growing bacteria as Salmonella will mostly invade tumor cells or cells of the tumor microenvironment and reside inside such cells, while extracellular accumulations might still occur. Bacterial counts of the recombinant Gram-negative bacterial strain accumulated inside the malignant solid tumor can be e.g. in the range of 10⁴ to 10⁹ bacteria per gram of tumor tissue.

The term “cancer” used herein refers to a disease in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. The term “cancer” used herein comprises solid tumors i.e. malignant solid tumors such as e.g. sarcomas, carcinomas, and lymphomas and non-solid tumors such as e.g. leukemias (cancers of the blood). Solid tumors are preferred.

The term “solid tumor” or “solid tumor indication” used herein refers to an abnormal mass of tissue that usually does not contain cysts or liquid areas. Solid tumors may be benign (not cancer), or malignant (cancer). Preferably malignant solid tumors are treated with the methods of the present invention. The term “malignant solid tumor” or “malignant solid tumor indication” used herein refers to an abnormal mass of tissue that usually does not contain cysts or liquid areas. Different types of malignant solid tumors are named for the type of cells that form them. Examples of malignant solid tumors are sarcomas, carcinomas, and lymphomas. Leukemias (cancers of the blood) generally do not form malignant solid tumors (definition according to the national cancer institute of the NIH). Malignant solid tumors include, but are not limited to, abnormal mass of cells which may stem from different tissue types such as liver, colon, colorectum, skin, breast, pancreas, cervix uteri, corpus uteri, bladder, gallbladder, kidney, larynx, lip, oral cavity, oesophagus, ovary, prostate, stomach, testis, thyroid gland or lung and thus include malignant solid liver, colon, colorectum, skin, breast, pancreas, cervix uteri, corpus uteri, bladder, gallbladder, kidney, larynx, lip, oral cavity, oesophagus, ovary, prostate, stomach, testis, thyroid gland or lung tumors. Preferred malignant solid tumors which can be treated with the methods of the present invention are malignant solid tumors which stem from skin, breast, liver, pancreas, bladder, prostate and colon and thus include malignant solid skin, breast, liver, pancreas, bladder, prostate and colon tumors. Equally preferred malignant solid tumors which can be treated with the methods of the present invention are malignant solid tumors associated with liver cancer, such as hepatocellular carcinoma.

The term “objective response rate” (ORR) as used herein refers to the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study. The ORR refers to the sum of complete response (CR) and partial response (PR). The definition of ORR, CR and PR for humans is provided in RECIST guidelines (RECIST 1.1; (Eisenhauer et al., 2009)) and adapted guidelines for assessment of immunotherapeutic compound (iRECIST; (Seymour et al., 2017))

In preclinical studies with tumour bearing mice, the definition of tumour response is adapted as compared to the RECIST definition for humans: no tumour regression is defined as tumour volumes increased by more than 35% compared to their respective volume at day 0; stable disease is defined as tumor volume change between 50% decrease and 35% increase of tumour volume compared to day 0; partial regression is defined as a decrease of tumour volume between 50% and 95% volume compared to day 0; and complete regression or complete response is defined as a decrease in tumour volume of >95% as compared to day 0.

The term “complete response” and “complete regression” are used herein interchangeably and have the same meaning. The term “complete response” (CR) in relation to target lesions refers to disappearance of all target lesions. Any pathological lymph nodes (whether target or non-target) must have reduction in short axis to <10 mm. The term complete response (CR) as used herein in relation to non-target lesions refers to disappearance of all non-target lesions and normalization of tumor marker level. All lymph nodes must be non-pathological in size (<10 mm short axis).

The term “partial response” (PR) as used herein in relation to target lesions refers to at least a 30% decrease in the sum of the diameters of target lesions, taking as reference the baseline sum diameters.

The term “progressive disease” (PD) as used herein in relation to target lesions refers to at least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. The appearance of one or more new lesions is also considered progressions. The term progressive disease (PD) as used herein in relation to non-target lesions refers to appearance of one or more new lesions and/or unequivocal progression of existing non-target lesions. Unequivocal progression should not normally trump target lesion status. It must be representative of overall disease status change, not a single lesion increase.

The term “stable disease” (SD) as used herein in relation to target lesions refers to neither sufficient shrinkage to qualify for PR nor sufficient increase to qualify for PD, taking as reference the smallest sum diameters while on study.

The term “progression-free survival” (PFS) as used herein relates to the duration of time from start of treatment to time of progression or death, whichever occurs first.

The term “bacterial effector protein which is virulent toward eukaryotic cells” as used herein refers to bacterial effector proteins, which are transported by secretion systems into host cells where they exert their virulence activity toward various host proteins and cellular machineries. Many different effector proteins are known, transported by various secretion system types and displaying a large repertoire of biochemical activities that modulate the functions of host regulatory molecules. Secretion systems include type 3 secretion system (T3SS), type 4 secretion system (T4SS) and type 6 secretion system (T6SS). Importantly, some effector proteins which are virulent toward eukaryotic cells (as Shigella flexneri IpaC) as well belong to the class of bacterial proteins, which are part of a secretion system machinery. In case the bacterial effector protein which is virulent toward eukaryotic cells is as well essential for the function of the secretion machinery, such a protein is excluded from this definition. T3SS effector proteins which are virulent towards eukaryotic cells refers to proteins as Y. enterocolitica YopE, YopH, YopJ, YopM, YopO, YopP, YopT or Shigella flexneri OspF, IpgD, IpgBI or Salmonella enterica SopE, SopB, SptP or P. aeruginosa ExoS, ExoT, ExoU, ExoY or E. coli Tir, Map, EspF, EspG, EspH, EspZ. T4SS effector proteins which are virulent towards eukaryotic cells refers to proteins as Legionella pneumophila LidA, SidC, SidG, SidH, SdhA, SidJ, SdjA, SdeA, SdeA, SdeC, LepA, LepB, WipA, WipB, YIfA, YIfB, VipA, VipF, VipD, VpdA, VpdB, DrrA, LegL3, LegL5, LegL7, LegLC4, LegLC8, LegC5, LegG2, Ceg10, Ceg23, Ceg29 or Bartonella henselae BepA, BepB, BepC, BepD, BepE, BepF BepG or Agrobacterium tumefaciens VirD2, VirE2, VirE3, VirF or H. pylori CagA or Bordetella pertussis pertussis toxin. T6SS effector proteins which are virulent towards eukaryotic cells refers to proteins as Vibrio cholerae VgrG proteins (as VgrG1).

The term “T3SS effector protein which is virulent toward eukaryotic cells” or “bacterial T3SS effector protein which is virulent toward eukaryotic cells” as used herein refers to proteins which are naturally injected by T3S systems into the cytosol of eukaryotic cells and to proteins which are naturally secreted by T3S systems that might e.g. form the translocation pore into the eukaryotic membrane, which are virulence factors toward eukaryotic cells i.e. to proteins which paralyze or reprogram the eukaryotic cell to the benefit of the pathogen. Effectors display a large repertoire of biochemical activities and modulate the function of crucial host regulatory mechanisms such as e.g. phagocytosis and the actin cytoskeleton, inflammatory signaling, apoptosis, endocytosis or secretory pathways (Cornelis, 2006; Mota and Cornelis, 2005) and include, but are not limited to, AvrA, AvrB, AvrBs2, AvrBS3, AvrBsT, AvrD, AvrD1, AvrPphB, AvrPphC, AvrPphEPto, AvrPpiBPto, AvrPto, AvrPtoB, AvrRpm1, AvrRpt2, AvrXv3, CigR, EspF, EspG, EspH, EspZ, ExoS, ExoT, GogB, GtgA, GtgE, GALA family of proteins, HopAB2, HopAO1, HopI1, HopM1, HopN1, HopPtoD2, HopPtoE, HopPtoF, HopPtoN, HopU1, HsvB, IcsB, IpaA, IpaH, IpaH7.8, IpaH9.8, IpgB1, IpgB2, IpgD, LcrV, Map, OspC1, OspE2, OspF, OspG, OspI, PipB, PipB2, PopB, PopP2, PthXo1, PthXo6, PthXo7, SifA, SifB, SipA/SspA, SlrP, SopA, SopB/SigD, SopD, SopE, SopE2, SpiC/SsaB, SptP, SpvB, SpvC, SrfH, SrfJ, Sse, SseB, SseC, SseD, SseF, SseG, SseI/SrfH, SseJ, SseK1, SseK2, SseK3, SseL, SspH1, SspH2, SteA, SteB, SteC, SteD, SteE, TccP2, Tir, VirA, VirPphA, VopF, XopD, YopE, YopH, YopJ, YopM, YopO, YopP, YopT, YpkA.

T3SS effector genes of Yersinia which are virulent to a eukaryotic cell and can be deleted/mutated from e.g. Y. enterocolitica are YopE, YopH, YopM, YopO, YopP (also named YopJ), and YopT (Trosky et al., 2008). The respective effector genes which are virulent to a eukaryotic cell can be deleted/mutated from Shigella flexneri (e.g. OspF, IpgD, IpgB1), Salmonella enterica (e.g. SopE, SopB, SptP), P. aeruginosa (e.g ExoS, ExoT, ExoU, ExoY) or E. coli (e.g. Tir, Map, EspF, EspG, EspH, EspZ). The nucleic acid sequences of these genes are available to those skilled in the art, e.g., in the Genbank Database (yopH, yopO, yopE, yopP, yopM, yopT from NC_002120 GI:10955536; S. flexneri effector proteins from AF386526.1 GI:18462515; S. enterica effectors from NC_016810.1 GI:378697983 or FQ312003.1 GI:301156631; P. aeruginosa effectors from AE004091.2 GI:110227054 or CP000438.1 GI:115583796 and E. coli effector proteins from NC_011601.1 GI:215485161).

For the purpose of the present invention, genes are denoted by letters of lower case and italicized to be distinguished from proteins. In case the genes (denoted by letters of lower case and italicized) are following a bacterial species name (like E. coli), they refer to a mutation of the corresponding gene in the corresponding bacterial species. For example, YopE refers to the effector protein encoded by the yopE gene. Y. enterocolitica yopE represents a Y. enterocolitica having a mutation in the yopE gene.

As used herein, the terms “polypeptide”, “peptide”, “protein”, “polypeptidic” and “peptidic” are used interchangeably to designate a series of amino acid residues connected to each other by peptide bonds between the alpha-amino and carboxy groups of adjacent residues. Preferred are proteins which have an amino acid sequence comprising at least 10 amino acids, more preferably at least 20 amino acids.

According to the present invention, “a heterologous protein or a fragment thereof” includes naturally occurring proteins or a fragment thereof and also includes artificially engineered proteins or a fragment thereof. Artificially engineered proteins or a fragment thereof are e.g. variants or functionally active fragments of the heterologous protein. By “variants or functionally active fragments thereof” in relation to the heterologous protein of the present invention is meant that the fragment or variant (such as an analogue, derivative or mutant) is capable of exercising the same physiological function as the heterologous protein. Such variants include naturally occurring allelic variants and non-naturally occurring variants. Additions, deletions, substitutions and derivatizations of one or more of the amino acids are contemplated so long as the modifications do not result in loss of functional activity of the fragment or variant. Preferably the functionally active fragment or variant has at least about 80% sequence identity more preferably at least about 90% sequence identity, even more preferably at least about 95% sequence identity, most preferably at least about 98% sequence identity to the relevant part of the heterologous protein. As used herein, the term “heterologous protein or a fragment thereof” refers to a protein or a fragment thereof other than the T3SS effector protein or N-terminal fragment thereof to which it can be fused. In particular the heterologous protein or a fragment thereof as used herein refers to a protein or a fragment thereof, which do not belong to the proteome, i.e. the entire natural protein complement of the specific recombinant Gram-negative bacterial strain provided and used by the invention, e.g. which do not belong to the proteome, i.e. the entire natural protein complement of a specific bacterial strain of the genera Yersinia, Escherichia, Salmonella or Pseudomonas. “Heterologous protein or a fragment thereof”, as used herein, is understood to mean that a gene or encoding sequence which codes for a protein or a fragment thereof which does not belong to the proteome i.e. the entire natural protein complement of the Gram-negative bacterial strain of the present invention, has been introduced into the Gram-negative bacterial strain by genetic transformation, transduction or conjugation. The heterologous protein or a fragment thereof can be located on a chromosome or on an extra-chromosomal genetic element of the Gram-negative bacterial strain. The gene or encoding sequence which codes for the heterologous protein or a fragment thereof originates from a source different from the host cell in which it is introduced. Usually the heterologous protein or a fragment thereof is of animal origin including human origin. Preferably the heterologous protein or a fragment thereof is a human protein or a fragment thereof. More preferably the heterologous protein or a fragment thereof is selected from the group consisting of proteins involved in induction or regulation of an interferon (IFN) response, proteins involved in apoptosis or apoptosis regulation, cell cycle regulators, ankyrin repeat proteins, cell signaling proteins, reporter proteins, transcription factors, proteases, small GTPases, GPCR related proteins, nanobody fusion constructs and nanobodies, bacterial T3SS effectors, bacterial T4SS effectors and viral proteins, or a fragment thereof. Particular preferably the heterologous protein or a fragment thereof is selected from the group consisting of proteins involved in induction or regulation of an interferon (IFN) response, proteins involved in apoptosis or apoptosis regulation, cell cycle regulators, ankyrin repeat proteins, reporter proteins, small GTPases, GPCR related proteins, nanobody fusion constructs, bacterial T3SS effectors, bacterial T4SS effectors and viral proteins, or a fragment thereof. Even more particular preferred are heterologous proteins or a fragment thereof selected from the group consisting of proteins involved in induction or regulation of an interferon (IFN) response, proteins involved in apoptosis or apoptosis regulation, cell cycle regulators, and ankyrin repeat proteins, or a fragment thereof. Most preferred are proteins or a fragment thereof involved in apoptosis or apoptosis regulation or proteins or a fragment thereof involved in induction or regulation of an interferon (IFN) response, in particular proteins or a fragment thereof involved in induction or regulation of an interferon (IFN) response, like animal, preferably human heterologous proteins or a fragment thereof involved in apoptosis or apoptosis regulation or human proteins or a fragment thereof involved in induction or regulation of an interferon (IFN) response. Proteins involved in induction or regulation of an interferon (IFN) response or a fragment thereof are preferably proteins involved in induction or regulation of a type I interferon (IFN) response or a fragment thereof, more preferably human proteins or a fragment thereof involved in induction or regulation of a type I interferon (IFN) response.

In some embodiments the Gram-negative bacterial strain of the present invention comprises two nucleotide sequences encoding the identical or two different heterologous proteins or a fragment thereof fused independently from each other in frame to the 3′end of a nucleotide sequence encoding a delivery signal from a bacterial effector protein.

In some embodiments the Gram-negative bacterial strain of the present invention comprises three nucleotide sequences encoding the identical or three different heterologous proteins or a fragment thereof fused independently from each other in frame to the 3′end of a nucleotide sequence encoding a delivery signal from a bacterial effector protein. In some embodiments the Gram-negative bacterial strain of the present invention comprises four nucleotide sequences encoding the identical or four different heterologous proteins or a fragment thereof fused independently from each other in frame to the 3′end of a nucleotide sequence encoding a delivery signal from a bacterial effector protein.

The heterologous protein expressed by the recombinant Gram-negative bacterial strain has usually a molecular weight of between 1 and 150 kDa, preferably between 1 and 120 kDa, more preferably between 1 and 100 kDa, most preferably between 10 and 80 kDa. A fragment of a heterologous protein contains usually between 10 and 1500 amino acids, preferably between 10 and 800 amino acids, more preferably between 100 and 800 amino acids, in particular between 100 and 500 amino acids. A fragment of a heterologous protein as defined herein does usually have the same functional properties as the heterologous protein from which it is derived.

In some embodiments a fragment of a heterologous protein comprises a domain of a heterologous protein. Thus in some embodiments the Gram-negative bacterial strain of the present invention comprises a nucleotide sequence encoding a domain of a heterologous protein. Preferably the Gram-negative bacterial strain of the present invention comprises a nucleotide sequence encoding one or two domains of a heterologous protein, more preferably two domains of a heterologous protein.

In some embodiments the Gram-negative bacterial strain of the present invention comprises a nucleotide sequence encoding repeated domains of a heterologous protein or two or more domains of different heterologous proteins fused in frame to the 3′end of a nucleotide sequence encoding a delivery signal from a bacterial effector protein.

The term “heterologous proteins which belong to the same functional class of proteins” as used herein refers to heterologous proteins which have the same function e.g. heterologous proteins having specific enzymatic activity, heterologous proteins which act in the same pathway such as e.g. cell cycle regulation, or share a common specific feature as e.g. belonging to the same class of bacterial effector proteins. Functional classes of proteins are e.g. proteins involved in apoptosis or apoptosis regulation, proteins which act as cell cycle regulators, ankyrin repeat proteins, cell signaling proteins, proteins involved in induction or regulation of an interferon (IFN) response, reporter proteins, transcription factors, proteases, small GTPases, GPCR related proteins, nanobody fusion constructs and nanobodies, bacterial T3SS effectors, bacterial T4SS effectors or viral proteins which act jointly in the biological process of establishing virulence to eukaryotic cells.

According to the present invention, “a domain of a heterologous protein” includes domains of naturally occurring proteins and also includes domains of artificially engineered proteins. As used herein, the term “domain of a heterologous protein” refers to a domain of a heterologous protein other than a domain of a T3SS effector protein or a domain other than a domain comprising the N-terminal fragment thereof to which it can be fused to achieve a fusion protein. In particular the domain of a heterologous protein as used herein refers to a domain of a heterologous protein, which do not belong to the proteome, i.e. the entire natural protein complement of the specific recombinant Gram-negative bacterial strain provided and used by the invention, e.g. which do not belong to the proteome, i.e. the entire natural protein complement of a specific bacterial strain of the genera Yersinia, Escherichia, Salmonella or Pseudomonas. Usually the domain of the heterologous protein is of animal origin including human origin. Preferably the domain of the heterologous protein is a domain of a human protein. More preferably the domain of the heterologous protein is a domain of a protein selected from the group consisting of proteins involved in apoptosis or apoptosis regulation, proteins involved in induction or regulation of an interferon (IFN) response, cell cycle regulators, ankyrin repeat proteins, cell signaling proteins, reporter proteins, transcription factors, proteases, small GTPases, GPCR related proteins, nanobody fusion constructs and nanobodies, bacterial T3SS effectors, bacterial T4SS effectors and viral proteins. Particular preferably the domain of the heterologous protein is a domain of a protein selected from the group consisting of proteins involved in apoptosis or apoptosis regulation, proteins involved in induction or regulation of an interferon (IFN) response, cell cycle regulators, ankyrin repeat proteins, reporter proteins, small GTPases, GPCR related proteins, nanobody fusion constructs, bacterial T3SS effectors, bacterial T4SS effectors and viral proteins. Even more particular preferred are domains of heterologous proteins selected from the group consisting of proteins involved in apoptosis or apoptosis regulation, proteins involved in induction or regulation of an interferon (IFN) response, cell cycle regulators, and ankyrin repeat proteins. Most preferred are domains of proteins involved in induction or regulation of an interferon (IFN) response, like animal proteins involved in induction or regulation of an interferon (IFN) response, preferably domains of human heterologous proteins involved in induction or regulation of an interferon (IFN) response, in particular domains of human heterologous proteins involved in induction or regulation of a type 1 interferon (IFN) response.

The domain of a heterologous protein expressed by the recombinant Gram-negative bacterial strain has usually a molecular weight of between 1-50 kDa, preferably between 1-30 kDa, more preferably between 1-20 kDa, most preferably between 1-15 kDa.

According to the present invention “proteins involved in induction or regulation of an IFN response” are heterologous proteins which when present in a mammalian cell e.g. when translocated by the recombinant Gram-negative bacterial strain of the present invention to a mammalian cell, trigger or participate in a signaling event or signaling cascade resulting in expression or altered expression, preferably in increased expression of IFNs by the mammalian cell. Proteins involved in induction or regulation of an IFN response include, but are not limited to, STING, TRIF, TBK1, IKKepsilon, IRF3, TREX1, VPS34, ATG9a, DDX3, LC3, DDX41, IFI16, MRE11, DNA-PK, RIG1 (DDX58), MDA5, LGP2, IPS-1/MAVS/Cardif/VISA, Trim25, Trim32, Trim56, Riplet, TRAF2, TRAF3, TRAF5, TANK, IRF3, IRF7, IRF9, STAT1, STAT2, PKR, TLR3, TLR7, TLR9, DAI, IFI16, IFIX, MRE11, DDX41, LSm14A, LRRFIP1, DHX9, DHX36, DHX29, DHX15, Ku70, IFNAR1, IFNAR2, TYK2, JAK1, ISGF3, IL10R2, IFNLR1, IFNGR1, IFNGR2, JAK2, STAT4, cyclic dinucleotide generating enzymes (cyclic-di-AMP cyclases, cyclic-di-GMP cyclases and cyclic-di-GAMP cyclases) as WspR, DncV, DisA and DisA-like, CdaA, CdaS and cGAS, or a fragment thereof.

According to the present invention “proteins involved in induction or regulation of a type I IFN response” are heterologous proteins which when present in a mammalian cell e.g. when translocated by the recombinant Gram-negative bacterial strain of the present invention to a mammalian cell, trigger or participate in a signaling event or signaling cascade resulting in expression or altered expression, preferably in increased expression of type I IFNs by the mammalian cell. Proteins involved in induction or regulation of a type I IFN response include, but are not limited to, STING, TRIF, TBK1, IKKepsilon, IRF3, TREX1, VPS34, ATG9a, DDX3, LC3, DDX41, IFI16, MRE11, DNA-PK, RIG1, MDA5, LGP2, IPS-1/MAVS/Cardif/VISA, Trim25, Trim32, Trim56, Riplet, TRAF2, TRAF3, TRAF5, TANK, IRF3, IRF7, IRF9, STAT1, STAT2, PKR, TLR3, TLR7, TLR9, DAI, IFI16, IFIX, MRE11, DDX41, LSm14A, LRRFIP1, DHX9, DHX36, DHX29, DHX15, Ku70, cyclic dinucleotide generating enzymes (cyclic-di-AMP cyclases, cyclic-di-GMP cyclases and cyclic-di-GAMP cyclases) as WspR, DncV, DisA and DisA-like, CdaA, CdaS and cGAS, or a fragment thereof.

Preferred proteins involved in induction or regulation of a type I IFN response are selected from the group consisting of STING, TRIF, TBK1, IKKepsilon, IRF3, TREX1, VPS34, ATG9a, DDX3, LC3, DDX41, IFI16, MRE11, DNA-PK, RIG1, MDA5, LGP2, IPS-1/MAVS/Cardif/VISA, Trim25, Trim32, Trim56, Riplet, TRAF2, TRAF3, TRAF5, TANK, IRF3, IRF7, IRF9, STAT1, STAT2, PKR, LSm14A, LRRFIP1, DHX29, DHX15, and cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, or a fragment thereof.

More preferred proteins involved in induction or regulation of a type I IFN response are selected from the group consisting of cGAS (as Uniprot. Q8N884 for the human protein), RIG1 (as Uniprot. O95786 for the human protein), MDA5 (as Uniprot. Q9BYX4 for the human protein), IPS-1/MAVS (as Uniprot. Q7Z434 for the human protein), IRF3 (as Uniprot. Q14653 for the human protein), IRF7 (as Uniprot. Q92985 for the human protein), IRF9 (as Uniprot. Q00978 for the human protein) and cyclic dinucleotide generating enzymes such as cyclic-di-AMP cyclases, cyclic-di-GMP cyclases and cyclic-di-GAMP cyclases selected from the group consisting of WspR (as Uniprot. Q9HXT9 for the P. aeruginosa protein), DncV (as Uniprot. Q9KVG7 for the V. cholerae protein), DisA and DisA-like (as Uniprot. Q812L9 for the B. cereus protein), CdaA (as Uniprot. Q8Y5E4 for the L. monocytogenes protein), CdaS (as Uniprot. 031854 or constitutive active L44F mutation for the B. subtilis protein) and cGAS (as Uniprot. Q8N884 for the human protein) or a fragment of these proteins.

IPS-1/MAVS/Cardif/VISA refer to the eukaryotic mitochondrial antiviral-signaling protein containing an N-terminal CARD domain and with the Uniprot (www.uniprot.org) identifier for the human sequence “Q7Z434” and “Q8VCF0” for the murine sequence. The terms “IPS-1/MAVS”, “MAVS/IPS-1” and “MAVS” are used herein interchangeably and refer to the eukaryotic mitochondrial antiviral-signaling protein containing an N-terminal CARD domain and with the Uniprot (www.uniprot.org) identifier for the human sequence “Q7Z434” and “Q8VCF0” for the murine sequence.

In some embodiments the heterologous proteins involved in induction or regulation of a type I IFN response are selected from the group consisting of a CARD domain containing proteins or a fragment thereof and cyclic dinucleotide generating enzymes such as cyclic-di-AMP cyclases, cyclic-di-GMP cyclases and cyclic-di-GAMP cyclases, or a fragment thereof. CARD domain containing heterologous proteins involved in induction or regulation of a type I IFN response are e.g. RIG1, which normally contains two CARD domains, MDA5 which normally contains two CARD domains, and MAVS which normally contains one CARD domain.

A fragment of a heterologous proteins involved in induction or regulation of a IFN response or a type I IFN response contains usually between 25 and 1000 amino acids, preferably between 50 and 600 amino acids, more preferably between 100 and 500 amino acids, even more preferably between 100 and 362 amino acids. In some embodiments a fragment of a heterologous proteins involved in induction or regulation of a IFN response or a type I IFN response comprises a fragment of the heterologous proteins involved in induction or regulation of a IFN response or a type I IFN response which contains usually between 25 and 1000 amino acids, preferably between 50 and 600 amino acids, more preferably between 100 and 500 amino acids, even more preferably between 100 and 362 amino acids, in particular between 100 and 246 amino acids or, comprises a fragment of the heterologous protein involved in induction or regulation of a IFN response or a type I IFN response which has a deletion of an amino acid sequence containing between amino acid 1 and amino acid 160 of the N-terminal amino acids, preferably a deletion of an amino acid sequence containing N-terminal amino aids 1-59 or N-terminal amino aids 1-160, and wherein the fragment of the heterologous protein involved in induction or regulation of a IFN response or a type I IFN response contains usually between 25 and 1000 amino acids, preferably between 50 and 600 amino acids, more preferably between 100 and 500 amino acids, even more preferably between 100 and 362 amino acids.

A fragment of a CARD domain containing heterologous proteins involved in induction or regulation of a IFN response or a type I IFN response contains usually an amino acid sequence from N-terminal amino acid 1 to any of amino acid 100-500, preferably an amino acid sequence from N-terminal amino acid 1 to any of amino acid 100-400, more preferably an amino acid sequence from N-terminal amino acid 1 to any of amino acid 100-300, more preferably an amino acid sequence from N-terminal amino acid 1 to any of amino acid 100-294, more preferably an amino acid sequence from N-terminal amino acid 1 to any of amino acid 100-246.

In some embodiments a fragment of a CARD domain containing heterologous proteins involved in induction or regulation of a IFN response or a type I IFN response contains an amino acid sequence selected from the group consisting of an amino acid sequence comprising at least N-terminal amino acid 1 and no more than amino acid 294, an amino acid sequence comprising at least N-terminal amino acid 1 and no more than amino acid 246, an amino acid sequence comprising at least N-terminal amino acid 1 and no more than amino acid 245, an amino acid sequence comprising at least N-terminal amino acid 1 and no more than amino acid 231, an amino acid sequence comprising at least N-terminal amino acid 1 and no more than amino acid 229, an amino acid sequence comprising at least N-terminal amino acid 1 and no more than amino acid 228, an amino acid sequence comprising at least N-terminal amino acid 1 and no more than amino acid 218, an amino acid sequence comprising at least N-terminal amino acid 1 and no more than amino acid 217, an amino acid sequence comprising at least N-terminal amino acid 1 and no more than amino acid 100 and an amino acid sequence comprising at least N-terminal amino acid 1 and no more than amino acid 101, more particular an amino acid sequence selected from the group consisting of an amino acid sequence comprising at least N-terminal amino acid 1 and no more than amino acid 245, an amino acid sequence comprising at least N-terminal amino acid 1 and no more than amino acid 228, an amino acid sequence comprising at least N-terminal amino acid 1 and no more than amino acid 217 and an amino acid sequence comprising at least N-terminal amino acid 1 and no more than amino acid 100, most particular an amino acid sequence comprising at least N-terminal amino acid 1 and no more than amino acid 245, of a CARD domain containing heterologous protein, preferably of a human CARD domain containing heterologous protein.

In some preferred embodiments the heterologous protein is a fragment of a CARD domain containing heterologous protein involved in induction or regulation of a IFN response or a type I IFN response or comprises a fragment of a CARD domain containing heterologous protein involved in induction or regulation of a IFN response or a type I IFN response. Usually the fragment of a CARD domain containing heterologous protein involved in induction or regulation of a IFN response or a type I IFN response comprises at least one CARD domain. In these embodiments the heterologous protein in particular contains or consists of an amino acid sequence selected from the group consisting of an amino acid sequence from N-terminal amino acid 1 to amino acid 294, an amino acid sequence from N-terminal amino acid 1 to amino acid 246, an amino acid sequence from N-terminal amino acid 1 to amino acid 245, an amino acid sequence from N-terminal amino acid 1 to amino acid 231, an amino acid sequence from N-terminal amino acid 1 to amino acid 229, an amino acid sequence from N-terminal amino acid 1 to amino acid 228, an amino acid sequence from N-terminal amino acid 1 to amino acid 218, an amino acid sequence from N-terminal amino acid 1 to amino acid 217, an amino acid sequence from N-terminal amino acid 1 to amino acid 100, an amino acid sequence from N-terminal amino acid 1 to amino acid 101, more particular an amino acid sequence selected from the group consisting of an amino acid sequence from N-terminal amino acid 1 to amino acid 245, an amino acid sequence from N-terminal amino acid 1 to amino acid 228, an amino acid sequence from N-terminal amino acid 1 to amino acid 217 and an amino acid sequence from N-terminal amino acid 1 to amino acid 100 of a heterologous protein involved in induction or regulation of a IFN response or a type I IFN response containing a CARD domain.

In these embodiments the heterologous protein more particular contains or consists of an amino acid sequence selected from the group consisting of an amino acid sequence from N-terminal amino acid 1 to amino acid 246, an amino acid sequence from N-terminal amino acid 1 to amino acid 245, an amino acid sequence from N-terminal amino acid 1 to amino acid 229, an amino acid sequence from N-terminal amino acid 1 to amino acid 228, an amino acid sequence from N-terminal amino acid 1 to amino acid 218, and an amino acid sequence from N-terminal amino acid 1 to amino acid 217, in particular an amino acid sequence from N-terminal amino acid 1 to amino acid 245, an amino acid sequence from N-terminal amino acid 1 to amino acid 228, an amino acid sequence from N-terminal amino acid 1 to amino acid 217, most particular an amino acid sequence from N-terminal amino acid 1 to amino acid 245, of RIG-1, or an amino acid sequence selected from the group consisting of an amino acid sequence from N-terminal amino acid 1 to amino acid 100, and an amino acid sequence from N-terminal amino acid 1 to amino acid 101 of MAVS, or an amino acid sequence selected from the group consisting of an amino acid sequence from N-terminal amino acid 1 to amino acid 294 and an amino acid sequence from N-terminal amino acid 1 to amino acid 231 of MDA5, even more particular an amino acid sequence selected from the group consisting of an an amino acid sequence from N-terminal amino acid 1 to amino acid 245, an amino acid sequence from N-terminal amino acid 1 to amino acid 228, and an amino acid sequence from N-terminal amino acid 1 to amino acid 217 of RIG-1, or an amino acid sequence from N-terminal amino acid 1 to amino acid 100, of MAVS, or an amino acid sequence selected from the group consisting of an amino acid sequence from N-terminal amino acid 1 to amino acid 294 and an amino acid sequence from N-terminal amino acid 1 to amino acid 231 of MDA5. Most preferred are the amino acid sequence from N-terminal amino acid 1 to amino acid 245 of human RIG-1 and the amino acid sequence from N-terminal amino acid 1 to amino acid 246 of murine RIG-1. The human RIG-1 1-245 fragment and the murine RIG-1 1-246 fragment correspond to each other by a sequence identity of 73% (and sequence similarity of 85%) and they are functionally equivalent i.e. both fragments show equivalent activity in murine cells and human cells.

In some preferred embodiments the heterologous protein is a fragment of cyclic dinucleotide generating enzymes such as cyclic-di-AMP cyclases, cyclic-di-GMP cyclases and cyclic-di-GAMP cyclases. A fragment of cyclic dinucleotide generating enzymes such as cyclic-di-AMP cyclases, cyclic-di-GMP cyclases and cyclic-di-GAMP cyclases contains usually an amino acid sequence from N-terminal amino acid 1 to any of amino acid 100-600, preferably an amino acid sequence from amino acid 50 to any of amino acid 100-550, more preferably an amino acid sequence from amino acid 60 to any of amino acid 100-530, in particular an amino acid sequence from amino acid 60 to amino acid 530, more particular an amino acid sequence from amino acid 146 to amino acid 507 or an amino acid sequence from amino acid 161 to amino acid 522, most particular an amino acid sequence from amino acid 161 to amino acid 522 of the cyclic dinucleotide generating enzymes, preferably of the human cGAS. In some embodiments a fragment of cGAS contains in particular an amino acid sequence selected from the group consisting of an amino acid sequence comprising at least amino acid 60 and no more than amino acid 422, an amino acid sequence comprising at least amino acid 146 and no more than amino acid 507, and an amino acid sequence comprising at least amino acid 161 and no more than amino acid 522. In some embodiments a fragment of cGAS contains more particular an amino acid sequence selected from the group consisting of an amino acid sequence from amino acid 60 to amino acid 422, an amino acid sequence from amino acid 146 to amino acid 507, and an amino acid sequence from amino acid 161 to amino acid 522, most preferably an amino acid sequence from amino acid 161 to amino acid 522.

In a more preferred embodiment the heterologous protein or a fragment thereof is a protein involved in induction or regulation of a type I IFN response selected from the group consisting of a CARD domain comprising RIG1, MDA5, and MAVS or a fragment thereof, wherein the fragment comprises at least one CARD domain, and cGAS and a fragment thereof, in particular selected from the group consisting of a CARD domain comprising RIG1 and a fragment thereof, wherein the fragment comprises at least one CARD domain, a CARD domain comprising MAVS and a fragment thereof, wherein the fragment comprises at least one CARD domain, and cGAS and a fragment thereof. Fragments of these proteins as outlined supra are particular preferred. In this more preferred embodiment, a CARD domain comprising RIG1, MDA5, MAVS comprises the naturally occurring CARD domain(s) and optionally additionally C-terminal amino acids following the naturally occurring CARD domain(s) e.g. comprising the naturally occurring helicase domain in case of RIG-1 or a fragment thereof, preferably a fragment containing 1-500, more preferably 1-250, wherein the naturally occurring helicase domain or fragment thereof is not functional, i.e. does not bind a CARD domain or, comprises optionally the downstream C-terminal sequence in case of MAVS or a fragment thereof, preferably a fragment containing 1-500, more preferably 1-250, even more preferably 1-150 amino acids. In these embodiments cGAS and a fragment thereof comprises usually the naturally occurring synthase domain (NTase core and C-terminal domain; amino acids 160-522 of the human cGAS as described in (Kranzusch et al., 2013) and as Uniprot. Q8N884 for the human protein), preferably cGAS and a fragment thereof comprises the naturally occurring synthase domain, but has a deletion of a part or the complete N-terminal domain, preferably a deletion of the complete N-terminal helical extension (N-terminal helical extension; amino acids 1-160 of the human cGAS as described in (Kranzusch et al., 2013) and as Uniprot. Q8N884 for the human protein). The deletion of a part or the complete N-terminal domain is preferably a deletion of the amino acids 1-160.

In a preferred embodiment the heterologous protein or a fragment thereof is a protein involved in induction or regulation of a type I IFN response selected from the group consisting of the RIG-I-like receptor (RLR) family (as RIG1 and MDA5) or a fragment thereof, other CARD domain containing proteins involved in antiviral signaling and type I IFN induction (as MAVS) or a fragment thereof and cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, or a fragment thereof. Cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, or a fragment thereof lead to stimulation of STING.

In some embodiments the heterologous protein or a fragment thereof is a protein involved in induction or regulation of a type I IFN response selected from the group consisting of RIG1, MDA5, LGP2, MAVS, WspR, DncV, DisA and DisA-like, CdaA, CdaS and cGAS, or a fragment thereof, more preferably selected from the group consisting of RIG1, MAVS, MDA5, WspR, DncV, DisA-like, and cGAS, or a fragment thereof, most preferably selected from the group consisting of RIG1 or a fragment thereof and cGAS, or a fragment thereof.

In a more preferred embodiment the protein involved in induction or regulation of a type I IFN response is selected from the group consisting of RIG1, MDA5, MAVS, WspR, DncV, DisA and DisA-like, CdaA, and cGAS, or a fragment thereof, even more preferably selected from the group consisting of RIG1, MDA5, MAVS, WspR, DncV, DisA-like, CdaA, and cGAS, or a fragment thereof, in particular selected from the group consisting of RIG1, MDA5, MAVS and cGAS, or a fragment thereof. Fragments of these proteins as described supra are particular preferred.

In this more preferred embodiment a fragment of RIG1, MDA5, MAVS usually contains an amino acid sequence from N-terminal amino acid 1 to any of amino acid 100-500, preferably an amino acid sequence from N-terminal amino acid 1 to any of amino acid 100-400, more preferably an amino acid sequence from N-terminal amino acid 1 to any of amino acid 100-300.

In this more preferred embodiment a fragment of RIG1 contains an amino acid sequence selected from the group consisting of an amino acid sequence comprising at least N-terminal amino acid 1 and no more than amino acid 246, an amino acid sequence comprising at least N-terminal amino acid 1 and no more than amino acid 245, an amino acid sequence comprising at least N-terminal amino acid 1 and no more than amino acid 229, an amino acid sequence comprising at least N-terminal amino acid 1 and no more than amino acid 228, an amino acid sequence comprising at least N-terminal amino acid 1 and no more than amino acid 218, and an amino acid sequence comprising at least N-terminal amino acid 1 and no more than amino acid 217, in particular an amino acid sequence comprising at least N-terminal amino acid 1 and no more than amino acid 245; a fragment of MDA5 contains an amino acid sequence selected from the group consisting of an amino acid sequence comprising at least N-terminal amino acid 1 and no more than amino acid 294, an amino acid sequence comprising at least N-terminal amino acid 1 and no more than amino acid 231, and a fragment of MAVS contains an amino acid sequence selected from the group consisting of an amino acid sequence comprising at least N-terminal amino acid 1 and no more than amino acid 100 and an amino acid sequence comprising at least N-terminal amino acid 1 and no more than amino acid 101.

In this more preferred embodiment a fragment of RIG1 contains more particular an amino acid sequence selected from the group consisting of an amino acid sequence from N-terminal amino acid 1 to amino acid 246, an amino acid sequence from N-terminal amino acid 1 to amino acid 245, an amino acid sequence from N-terminal amino acid 1 to amino acid 229, an amino acid sequence from N-terminal amino acid 1 to amino acid 228, an amino acid sequence from N-terminal amino acid 1 to amino acid 218, and an amino acid sequence from N-terminal amino acid 1 to amino acid 217, even more particular an amino acid sequence from N-terminal amino acid 1 to amino acid 245, an amino acid sequence from N-terminal amino acid 1 to amino acid 228, an amino acid sequence from N-terminal amino acid 1 to amino acid 217, most particular an amino acid sequence from N-terminal amino acid 1 to amino acid 245; a fragment of MDA5 contains more particular an amino acid sequence selected from the group consisting of an amino acid sequence from N-terminal amino acid 1 to amino acid 294 and an amino acid sequence from N-terminal amino acid 1 to amino acid 231; and a fragment of MAVS contains more particular an amino acid sequence selected from the group consisting of amino acid sequence from N-terminal amino acid 1 to amino acid 100 and an amino acid sequence from N-terminal amino acid 1 to amino acid 101.

In this more preferred embodiment a fragment of cGAS contains usually an amino acid sequence from N-terminal amino acid 1 to any of amino acid 100-600, preferably an amino acid sequence from amino acid 50 to any of amino acid 100-550, more preferably an amino acid sequence from amino acid 60 to any of amino acid 100-530, in particular an amino acid sequence from amino acid 60 to amino acid 530, an amino acid sequence from amino acid 146 to amino acid 507 or an amino acid sequence from amino acid 161 to amino acid 530, more particular an amino acid sequence from amino acid 60 to amino acid 530, or an amino acid sequence from amino acid 161 to amino acid 530 of the human cGAS.

In this more preferred embodiment a fragment of cGAS contains in particular an amino acid sequence selected from the group consisting of an amino acid sequence comprising at least amino acid 60 and no more than amino acid 422, an amino acid sequence comprising at least amino acid 146 and no more than amino acid 507, and an amino acid sequence comprising at least amino acid 161 and no more than amino acid 522.

In this more preferred embodiment a fragment of cGAS contains more particular an amino acid sequence selected from the group consisting of an amino acid sequence from amino acid 60 to amino acid 422, an amino acid sequence from amino acid 146 to amino acid 507, an amino acid sequence from amino acid 161 to amino acid 522, most particular an amino acid sequence from amino acid 161 to amino acid 522.

In an even more preferred embodiment the protein involved in induction or regulation of a type I IFN response is selected from the group consisting of human RIG1 CARD domains₁₋₂₄₅ (SEQ ID NO: 1), human RIG1 CARD domains₁₋₂₂₈ (SEQ ID NO: 2), human RIG1 CARD domains₁₋₂₁₇ (SEQ ID NO: 3), murine RIG1 CARD domains₁₋₂₄₆ (SEQ ID NO: 4), murine RIG1 CARD domains₁₋₂₂₉ (SEQ ID NO: 5), murine RIG1 CARD domains₁₋₂₁₈ (SEQ ID NO: 6), human MAVS CARD domain 1-100 (SEQ ID NO: 7), murine MAVS CARD domain₁₋₁₀₁ (SEQ ID NO: 8), N. vectensis cGAS (SEQ ID NO: 9), human cGAS₁₆₁₋₅₂₂ (SEQ ID NO: 10), murine cGAS₁₄₆₋₅₀₇ (SEQ ID NO: 11), N. vectensis cGAS₆₀₋₄₂₂ (SEQ ID NO: 12), murine MDA5₁₋₂₉₄ (SEQ ID NO: 13), murine MDA5₁₋₂₃₁ (SEQ ID NO: 14), human MDA5₁₋₂₉₄ (SEQ ID NO: 15), and human MDA5₁₋₂₃₁ (SEQ ID NO: 16).

In a particular preferred embodiment the protein involved in induction or regulation of a type I IFN response is selected from the group consisting of human RIG1 CARD domains₁₋₂₄₅, (SEQ ID NO: 1), human RIG1 CARD domains₁₋₂₂₈ (SEQ ID NO: 2), human RIG1 CARD domains₁₋₂₁₇ (SEQ ID NO: 3), human MAVS CARD domain₁₋₁₀₀ (SEQ ID NO: 7), and human cGAS₁₆₁₋₅₂₂ (SEQ ID NO: 10).

In a more particular preferred embodiment the protein involved in induction or regulation of a type I IFN response is selected from the group consisting of human RIG1 CARD domains₁₋₂₄₅ (SEQ ID NO: 1), murine RIG1 CARD domains₁₋₂₄₆ (SEQ ID NO: 4), murine RIG1 CARD domains₁₋₂₂₉ (SEQ ID NO: 5), murine RIG1 CARD domains₁₋₂₁₈ (SEQ ID NO: 6), and human cGAS₁₆₁₋₅₂₂ (SEQ ID NO: 10), most particular selected from the group consisting of human RIG1 CARD domains₁₋₂₄₅ (SEQ ID NO: 1) and human cGAS₁₆₁₋₅₂₂ (SEQ ID NO: 10).

The RIG-I-like receptor (RLR) family comprises proteins selected from the group consisting of RIG1, MDA5 and LGP2. Preferred heterologous proteins involved in induction or regulation of a type I IFN response are the CARD domain containing proteins RIG1 and MDA5, in particular the CARD domain containing protein RIG1. Other preferred CARD domain containing proteins involved in type I IFN induction comprises proteins selected form the group consisting of MAVS.

In some preferred embodiments the heterologous proteins involved in induction or regulation of a type I IFN response are selected from the group of proteins comprising a CARD domain of RIG1, a CARD domain of MDA5, and/or a CARD domain of MAVS, and WspR, DncV, DisA and DisA-like, CdaA, CdaS and cGAS and a fragment thereof, preferably selected from the group of proteins comprising of a CARD domain of RIG1, a CARD domain of MDA5 and/or a CARD domain of MAVS, and WspR, DncV, DisA and DisA-like, CdaA, and cGAS, or a fragment thereof.

In some preferred embodiments the heterologous proteins involved in induction or regulation of a type I IFN response are selected from the group consisting of a CARD domain of RIG1, a CARD domain of MDA5, a CARD domain of MAVS, WspR, DncV, DisA and DisA-like, CdaA, CdaS and cGAS, more preferably selected from the group consisting of a CARD domain of RIG1, WspR, DncV, DisA-like, and cGAS.

In some preferred embodiments the heterologous proteins involved in induction or regulation of a type I IFN response comprises one or more (e.g. two, three or four) CARD domains, preferably comprises one or more (e.g. two, three or four) CARD domains of RIG1, MDA5, and/or MAVS, preferably of RIG1 and/or MAVS. In a more preferred embodiment the heterologous proteins involved in induction or regulation of a type I IFN response comprises both CARD domains of RIG1, both CARD domains of MDA5 and/or the CARD domain of MAVS and cGAS or a fragment thereof, in particular both CARD domains of RIG1 and cGAS or a fragment thereof, more particular both CARD domains of RIG1.

In some embodiments the heterologous proteins involved in induction or regulation of a type I IFN response are selected from the group consisting of a type I IFN response inducing protein without enzymatic function and a type I IFN response inducing protein with enzymatic function. A type I IFN response inducing protein without enzymatic function encompassed by the present invention comprise usually at least one CARD domain preferably two CARD domains. A CARD domain is normally composed of a bundle of six to seven alpha-helices, preferably an arrangement of six to seven antiparallel alpha helices with a hydrophobic core and an outer face composed of charged residues. A type I IFN response inducing protein with enzymatic function encompassed by the present invention comprise usually a cyclic dinucleotide generating enzyme (cyclic-di-AMP cyclases, cyclic-di-GMP cyclases and cyclic-di-GAMP cyclases) or a domain thereof leading to stimulation of STING, preferably a di-adenylate-cyclase (DAC), di-guanylate-cyclase (DGC) or GMP-AMP-cyclase (GAC) or domain thereof.

According to the present invention “proteins involved in apoptosis or apoptosis regulation” include, but are not limited to, Bad, Bcl2, Bak, Bmt, Bax, Puma, Noxa, Bim, Bcl-xL, Apaf1, Caspase 9, Caspase 3, Caspase 6, Caspase 7, Caspase 10, DFFA, DFFB, ROCK1, APP, CAD, ICAD, CAD, EndoG, AIF, HtrA2, Smac/Diablo, Arts, ATM, ATR, Bok/Mtd, Bmf, Mcl-1(S), IAP family, LC8, PP2B, 14-3-3 proteins, PKA, PKC, PI3K, Erk1/2, p90RSK, TRAF2, TRADD, FADD, Daxx, Caspase8, Caspase2, RIP, RAIDD, MKK7, JNK, FLIPs, FKHR, GSK3, CDKs and their inhibitors like the INK4-family (p16(Ink4a), p15(Ink4b), p18(Ink4c), p19(Ink4d)), and the Cip1/Waf1/Kip1-2-family (p21(Cip1/Waf1), p27(Kip1), p57(Kip2). Preferably Bad, Bmt, Bcl2, Bak, Bax, Puma, Noxa, Bim, Bcl-XL, Caspase9, Caspase3, Caspase6, Caspase7, Smac/Diablo, Bok/Mtd, Bmf, Mcl-1(S), LC8, PP2B, TRADD, Daxx, Caspase8, Caspase2, RIP, RAIDD, FKHR, CDKs and their inhibitors like the INK4-family (p16(Ink4a), p15(Ink4b), p18(Ink4c), p19(Ink4d)), most preferably BIM, Bid, truncated Bid, FADD, Caspase 3 (and subunits thereof), Bax, Bad, Akt, CDKs and their inhibitors like the INK4-family (p16(Ink4a), p15(Ink4b), p18(Ink4c), p19(Ink4d)) are used (Brenner and Mak, 2009; Chalah and Khosravi-Far, 2008; Fuchs and Steller, 2011). Additionally proteins involved in apoptosis or apoptosis regulation include DIVA, Bcl-Xs, Nbk/Bik, Hrk/Dp5, Bid and tBid, Egl-1, Bcl-Gs, Cytochrome C, Beclin, CED-13, BNIP1, BNIP3, Bcl-B, Bcl-W, Ced-9, A1, NR13, Bfl-1, Caspase 1, Caspase 2, Caspase 4, Caspase 5, Caspase 8.

Proteins involved in apoptosis or apoptosis regulation are selected from the group consisting of pro-apoptotic proteins, anti-apoptotic proteins, inhibitors of apoptosis-prevention pathways and inhibitors of pro-survival signalling or pathways. Pro-apoptotic proteins comprise proteins selected form the group consisting of Bax, Bak, Diva, Bcl-Xs, Nbk/Bik, Hrk/Dp5, Bmf, Noxa, Puma, Bim, Bad, Bid and tBid, Bok, Apaf1, Smac/Diablo, BNIP1, BNIP3, Bcl-Gs, Beclin 1, Egl-1 and CED-13, Cytochrome C, FADD, the Caspase family, and CDKs and their inhibitors like the INK4-family (p16(Ink4a), p15(Ink4b), p18(Ink4c), p19(Ink4d)) or selected from the group consisting of Bax, Bak, Diva, Bcl-Xs, Nbk/Bik, Hrk/Dp5, Bmf, Noxa, Puma, Bim, Bad, Bid and tBid, Bok, Egl-1, Apaf1, Smac/Diablo, BNIP1, BNIP3, Bcl-Gs, Beclin 1, Egl-1 and CED-13, Cytochrome C, FADD, and the Caspase family. Preferred are Bax, Bak, Diva, Bcl-Xs, Nbk/Bik, Hrk/Dp5, Bmf, Noxa, Puma, Bim, Bad, Bid and tBid, Bok, Egl-1, Apaf1, BNIP1, BNIP3, Bcl-Gs, Beclin 1, Egl-1 and CED-13, Smac/Diablo, FADD, the Caspase family, CDKs and their inhibitors like the INK4-family (p16(Ink4a), p15(Ink4b), p18(Ink4c), p19(Ink4d)). Equally preferred are Bax, Bak, Diva, Bcl-Xs, Nbk/Bik, Hrk/Dp5, Bmf, Noxa, Puma, Bim, Bad, Bid and tBid, Bok, Apaf1, BNIP1, BNIP3, Bcl-Gs, Beclin 1, Egl-1 and CED-13, Smac/Diablo, FADD, the Caspase family.

Anti-apoptotic proteins comprise proteins selected form the group consisting of Bcl-2, Bcl-Xl, Bcl-B, Bcl-W, Mcl-1, Ced-9, A1, NR13, IAP family and Bfl-1. Preferred are Bcl-2, Bcl-Xl, Bcl-B, Bcl-W, Mcl-1, Ced-9, A1, NR13 and Bfl-1.

Inhibitors of apoptosis-prevention pathways comprise proteins selected form the group consisting of Bad, Noxa and Cdc25A. Preferred are Bad and Noxa.

Inhibitors of pro-survival signalling or pathways comprise proteins selected form the group consisting of PTEN, ROCK, PP2A, PHLPP, JNK, p38. Preferred are PTEN, ROCK, PP2A and PHLPP.

In some embodiments, the heterologous protein involved in apoptosis or apoptosis regulation are selected from the group consisting of BH3-only proteins, caspases and intracellular signalling proteins of death receptor control of apoptosis or a fragment thereof. BH3-only proteins are preferred. BH3-only proteins comprise proteins selected form the group consisting of Bad, BIM, Bid and tBid, Puma, Bik/Nbk, Bod, Hrk/Dp5, BNIP1, BNIP3, Bmf, Noxa, Mcl-1, Bcl-Gs, Beclin 1, Egl-1 and CED-13. Preferred are Bad, BIM, Bid and tBid, in particular tBid.

Caspases comprise proteins selected form the group consisting of Caspase 1, Caspase 2, Caspase 3, Caspase 4, Caspase 5, Caspase 6, Caspase 7, Caspase 8, Caspase 9, Caspase 10. Preferred are Caspase 3, Caspase 8 and Caspase 9.

Intracellular signalling proteins of death receptor control of apoptosis comprise proteins selected form the group consisting of FADD, TRADD, ASC, BAP31, GULP1/CED-6, CIDEA, MFG-E8, CIDEC, RIPK1/RIP1, CRADD, RIPK3/RIP3, Crk, SHB, CrkL, DAXX, the 14-3-3 family, FLIP, DFF40 and 45, PEA-15, SODD. Preferred are FADD and TRADD.

In some embodiments two heterologous proteins involved in apoptosis or apoptosis regulation are comprised by the Gram-negative bacterial strain, wherein one protein is a pro-apoptotic protein and the other protein is an inhibitor of apoptosis-prevention pathways or wherein one protein is a pro-apoptotic protein and the other protein is an inhibitor of pro-survival signalling or pathways.

Pro-apoptotic proteins encompassed by the present invention have usually an alpha helical structure, preferably a hydrophobic helix surrounded by amphipathic helices and usually comprise at least one of BH1, BH2, BH3 or BH4 domains, preferably comprise at least one BH3 domain. Usually pro-apoptotic proteins encompassed by the present invention have no enzymatic activity.

Anti-apoptotic proteins encompassed by the present invention have usually an alpha helical structure, preferably a hydrophobic helix surrounded by amphipathic helices and comprises a combination of different BH1, BH2, BH3 and BH4 domains, preferably a combination of different BH1, BH2, BH3 and BH4 domains wherein a BH1 and a BH2 domain is present, more preferably BH4-BH3-BH1-BH2, BH1-BH2, BH4-BH1-BH2 or BH3-BH1-BH2 (from N- to the C-terminus). Additionally, proteins containing at least one BIR domain are also encompassed.

Inhibitors of apoptosis-prevention pathways encompassed by the present invention have usually an alpha helical structure, preferably a hydrophobic helix surrounded by amphipathic helices and usually comprise one BH3 domain.

BH1, BH2, BH3 or BH4 domains are each usually between about 5 to about 50 amino acids in length. Thus in some embodiments the heterologous proteins involved in apoptosis or apoptosis regulation is selected from the group consisting of heterologous proteins involved in apoptosis or apoptosis regulation which are about 5 to about 200, preferably about 5 to about 150, more preferably about 5 to about 100, most preferably about 5 to about 50, in particular about 5 to about 25 amino acids in length.

A particular preferred heterologous protein is the BH3 domain of apoptosis inducer tBID, more particular the BH3 domain comprising a sequence selected from the group consisting of SEQ ID NOs: 17-20, preferably SEQ ID NO: 17 or SEQ ID NO: 18.

Equally preferred is the BH3 domain of apoptosis regulator BAX, more particular the BAX domain comprising a sequence selected from the group consisting of SEQ ID NOs: 21-24, preferably SEQ ID NO: 21 or SEQ ID NO: 22. The human and murine sequences are given in SEQ ID NOs, but tBID and BAX BH3 domains of all other species are equally included.

Another particular preferred heterologous protein is a heterologous protein containing a domain of a protein involved in induction or regulation of a type I IFN response, more particular a heterologous protein containing a domain of a protein involved in induction or regulation of a type I IFN response selected from the group consisting of i) a CARD domain of RIG1 comprising a sequence selected from the group consisting of SEQ ID NOs: 1-6, ii) a CARD domain of MDA5 comprising a sequence selected from the group consisting of SEQ ID NOs: 13-16, preferably SEQ ID NOs: 15 or 16, and iii) a CARD domain of MAVS comprising a sequence selected from the group consisting of SEQ ID NO: 7 or 8, preferably SEQ ID NO: 7. Another particular preferred heterologous protein is a full-length cGAS such as N. vectensis cGAS (SEQ ID NO: 9), human cGAS₁₆₁₋₅₂₂ (SEQ ID NO: 10), N. vectensis cGAS₆₀)-422 (SEQ ID NO: 12) or murine cGAS₁₄₆₋₅₀₇ (SEQ ID NO: 11). Most particularly preferred heterologous protein are heterologous proteins containing a CARD domain of human RIG1 (SEQ ID NOs: 1-3), in particular a CARD domain of human RIG1 (SEQ ID NO: 1), and human cGAS₁₆₁₋₅₂₂ (SEQ ID NO: 10).

In some embodiments the heterologous proteins is a pro-drug converting enzyme. In these embodiments the recombinant Gram-negative bacterial strain expresses, preferably expresses and secretes a pro-drug converting enzyme. A prodrug converting enzyme as referred herein comprises enzymes converting non-toxic prodrugs into a toxic drug, preferably enzymes selected from the group consisting of cytosine deaminase, purine nucleoside phosphorylase, thymidine kinase, beta-galactosidase, carboxylesterases, nitroreductase, carboxypeptidases and beta-glucuronidases, more preferably enzymes selected from the group consisting of cytosine deaminase, purine nucleoside phosphorylase, thymidine kinase, and beta-galactosidase.

The term “protease cleavage site” as used herein refers to a specific amino acid motif within an amino acid sequence e.g. within an amino acid sequence of a protein or a fusion protein, which is cleaved by a specific protease, which recognizes the amino acid motif. For review see (Waugh, 2011). Examples of protease cleavage sites are amino acid motifs, which are cleaved by a protease selected from the group consisting of enterokinase (light chain), enteropeptidase, prescission protease, human rhinovirus protease (HRV 3C), TEV protease, TVMV protease, FactorXa protease and thrombin.

The following amino acid motif is recognized by the respective protease:
Asp-Asp-Asp-Asp-Lys: Enterokinase (light chain)/Enteropeptidase (SEQ ID NO: 37)
Leu-Glu-Val-Leu-Phe-Gln/Gly-Pro: PreScission Protease/human Rhinovirus protease (HRV 3C) (SEQ ID NO:38)
Glu-Asn-Leu-Tyr-Phe-Gln-Ser and modified motifs based on the Glu-X-X-Tyr-X-Gln-Gly/Ser (where X is any amino acid) recognized by TEV protease (tobacco etch virus) (SEQ ID NO: 39) and (SEQ ID NO: 40)
Glu-Thr-Val-Arg-Phe-Gln-Ser: TVMV protease (SEQ ID NO: 41)
Ile-(Glu or Asp)-Gly-Arg: FactorXa protease (SEQ ID NO: 42)
Leu-Val-Pro-Arg/Gly-Ser: Thrombin (SEQ ID NO: 43).

Encompassed by the protease cleavage sites as used herein is ubiquitin. Thus in some preferred embodiments ubiquitin is used as protease cleavage site, i.e. a nucleotide sequence encodes ubiquitin as protease cleavage site, which can be cleaved by a specific ubiquitin processing proteases at the N-terminal site, e.g. which can be cleaved by a specific ubiquitin processing proteases called Deubiquitinating enzymes at the N-terminal site endogenously in the cell where the fusion protein has been delivered to. Ubiquitin is processed at its C-terminus by a group of endogenous Ubiquitin-specific C-terminal proteases (Deubiquitinating enzymes, DUBs). The cleavage of Ubiquitin by DUBs is supposed to happen at the very C-terminus of Ubiquitin (after G76).

The term “mutation” is used herein as a general term and includes changes of both single base pair and multiple base pairs. Such mutations may include substitutions, frame-shift mutations, deletions, insertions and truncations.

The term “nuclear localization signal” as used herein refers to an amino acid sequence that marks a protein for import into the nucleus of a eukaryotic cell and includes preferably a viral nuclear localization signal such as the SV40 large T-antigen derived NLS (PPKKKRKV) (SEQ ID NO: 44).

The term “multiple cloning site” as used herein refers to a short DNA sequence containing several restriction sites for cleavage by restriction endonucleases such as AclI, HindIII, SspI, MluCI, Tsp5091, PciI, AgeI, BspMI, BfuAI, SexAI, MluI, BceAI, HpyCH4IV, HpyCH4III, BaeI, BsaXI, AflIII, SpeI, BsrI, BmrI, BgIII, AfeI, AluI, StuI, ScaI, ClaI, BspDI, PI-SceI, NsiI, AseI, SwaI, CspCI, MfeI, BssSI, BmgBI, PmlI, DraIII, AleI, EcoP15I, PvuII, AlwNI, BtsIMutI, TspRI, Ndel, NlaIII, CviAII, FatI, MslI, FspEI, XcmI, BstXI, PfIMI, BccI, NcoI, BseYI, FauI, SmaI, XmaI, TspMI, Nt.CviPII, LpnPI, AciI, SacII, BsrBI, MspI, HpaII, ScrFI, BssKI, StyD4I, BsaJI, BslI, BtgI, NciI, AvrII, MnlI, BbvCI, Nb.BbvCI, Nt.BbvCI, SbfI, Bpu10I, Bsu36I, EcoNI, HpyAV, BstNI, PspGI, StyI, BcgI, PvuI, BstUI, EagI, RsrII, BsiEI, BsiWI, BsmBI, Hpy99I, MspA1I, MspJI, SgrAI, BfaI, BspCNI, XhoI, EarI, AcuI, PstI, BpmI, DdeI, SfcI, AflII, BpuEI, SmlI, AvaI, BsoBI, MboII, BbsI, XmnI, BsmI, Nb.BsmI, EcoRI, HgaI, AatII, ZraI, Tth111I PflFI, PshAI, AhdI, DrdI, Eco53kI, SacI, BseRI, PleI, Nt.BstNBI, MlyI, HinfI, EcoRV, MboI, Sau3 AI, DpnII BfuCI, DpnI, BsaBI, TfiI, BsrDI, Nb.BsrDI, BbvI, BtsI, Nb.BtsI, BstAPI, SfaNI, SphI, NmeAIII, NaeI, NgoMIV, BglI, AsiSI, BtgZI, HinP1I, HhaI, BssHII, NotI, Fnu4HI, Cac8I, MwoI, NheI, BmtI, SapI, BspQI, Nt.BspQI, BlpI, TseI, ApeKI, Bsp1286I, AlwI, Nt.AlwI, BamHI, FokI, BtsCI, HaeIII, PhoI, FseI, SfiI, NarI, KasI, SfoI, PluTI, AscI, EciI, BsmFI, ApaI, PspOMI, Sau96I, NlaIV, KpnI, Acc65I, BsaI, HphI, BstEII, AvaII, BanI, BaeGI, BsaHI, BanII, RsaI, CviQI, BstZ17I, BciVI, SalI, Nt.BsmAI, BsmAI, BcoDI, ApaLI, BsgI, AccI, Hpy166II, Tsp45I, HpaI, PmeI, HincII, BsiHKAI, ApoI, NspI, BsrFI, BstYI, HaeII, CviKI-1, EcoO109I, PpuMI, I-CeuI, SnaBI, I-SceI, BspHI, BspEI, MmeI, TaqαI, NruI, Hpy188I, Hpy188III, XbaI, BclI, HpyCH4V, FspI, PI-PspI, MscI, BsrGI, MseI, PacI, PsiI, BstBI, DraI, PspXI, BsaWI, BsaAI, EaeI, preferably XhoI, XbaI, HindIII, NcoI, NotI, EcoRI, EcoRV, BamHI, NheI, SacI, SalI, BstBI. The term “multiple cloning site” as used herein further refers to a short DNA sequence used for recombination events as e.g in Gateway cloning strategy or for methods such as Gibbson assembly or topo cloning.

The term “wild type strain” or “wild type of the Gram-negative bacterial strain” as used herein refers to a naturally occurring variant or a naturally occurring variant containing genetic modifications allowing the use of vectors, such as deletion mutations in restriction endonucleases or antibiotic resistance genes. These strains contain chromosomal DNA as well as in some cases (e.g. Y. enterocolitica, S. flexneri) an unmodified virulence plasmid.

The term “Yersinia wild type strain” as used herein refers to a naturally occurring variant (as Y. enterocolitica E40) or a naturally occurring variant containing genetic modifications allowing the use of vectors, such as deletion mutations in restriction endonucleases or antibiotic resistance genes (as Y. enterocolitica MRS40, the Ampicillin sensitive derivate of Y. enterocolitica E40). These strains contain chromosomal DNA as well as an unmodified virulence plasmid (called pYV).

Y. enterocolitica subspecies palearctica refers to the low-pathogenic Y. enterocolitica strains, which are in contrast to the higher virulent strains of subspecies enterocolitica (Howard et al., 2006; Thomson et al., 2006). Y. enterocolitica subsp. palearctica lack, in comparison to Y. enterocolitica subsp. enterocolitica, a high-pathogenicity island (HPI). This HPI encodes the iron siderophore called yersiniabactin (Pelludat et al., 2002). The lack of yersiniabactin in Y. enterocolitica subsp. palearctica renders this subspecies less pathogenic and dependent on induced systemic accessible iron for persistent infection in e.g. liver or spleen (Pelludat et al., 2002). Iron can be made accessible for the bacteria in an individual e.g. by pretreatment with deferoxamine, an iron chelator used to treat iron overload in patients (Mulder et al., 1989).

The term “immune checkpoint” or “immune checkpoint molecule” as used herein refers to a negative or positive stimulus to dampen or promote the immune response which is triggered by a cognate HLA/antigen-T-cell receptor activation or other activation of the immune system. Immune checkpoint molecules are typically involved in immune pathways and, for example, regulate T-cell activation, T-cell proliferation and/or T-cell function. Many of the immune checkpoint molecules belong to the immunoglobulin super family, more specifically the B7-CD28 family, or to the tumor necrosis factor/tumor necrosis factor receptor (TNF/TNFR) superfamily and, by the binding of specific ligands, activate signaling molecules that are recruited to the cytoplasmic domain (Suzuki et al., 2016). Examples of immune checkpoints include PD-1 (programmed cell death protein 1, CD279). Terms in brackets refer to synonyms of corresponding proteins, which are indicated for convenience. CD279 is a synonym for PD-1, which thus is indicated as PD-1 (CD279). Listing of synonyms is not concluding and further synonyms exist for many immune checkpoints.

The term “immune checkpoint modulator (ICM)” as used herein refers to a molecule, i.e. a protein as an antibody, modulating an immune checkpoint. An immune checkpoint modulator (ICM) interferes with immune checkpoints and modulates the function of one or more immune checkpoint molecules. “Modulation” or “modulate” in relation to an ICM means herein that an ICM totally or partially reduces, inhibits, interferes with, activates, stimulates, increases, reinforces or supports the function of one or more immune checkpoint molecules. Thus, an immune checkpoint modulator may be an “immune checkpoint inhibitor” (also referred to as “checkpoint inhibitor” or “inhibitor”) or an “immune checkpoint activator” (also referred to as “checkpoint activator” or “activator”).

Examples of immune checkpoint modulators which allow to interfere with immune checkpoints such as PD-1 are immune checkpoint inhibitors (CPIs) such as anti-PD-1 antibodies which prevent or reduce the negative feedback of the corresponding negative feedback loop.

Immune checkpoint modulators are typically able to modulate (i) self-tolerance and/or (ii) the amplitude and/or the duration of the immune response. Preferably, the immune checkpoint modulator used according to the present invention modulates the function of one or more human checkpoint molecules and is, thus, a “human checkpoint modulator”. Accordingly, the function of immune checkpoints which are modulated (e.g., totally or partially reduced, inhibited, interfered with, activated, stimulated, increased, reinforced or supported) by checkpoint modulators, is typically the regulation of T-cell activation, T-cell proliferation and/or T cell function.

An “immune checkpoint inhibitor” (also referred to as “checkpoint inhibitor” or “inhibitor” herein) totally or partially reduces, inhibits, interferes with, or negatively affects the function of one or more checkpoint molecules.

An “immune checkpoint activator” (also referred to as “checkpoint activator” or “activator” herein) totally or partially activates, stimulates, increases, reinforces, supports or positively affects the function of one or more checkpoint molecules.

The term “PD-1 antagonist” or “PD-1 inhibitor” is used herein interchangeably and refers to a molecule e.g. an antibody like an antagonistic PD-1 antibody which totally or partially reduces, inhibits, interferes with, or negatively modulates the function of PD1. A PD-1 antagonist is preferably a PD-1-antagonist which totally or partially reduces, inhibits, interferes with, or negatively modulates the function of PD-1 by interfering with PD-L2 and/or PD-L1 binding, like an antagonistic PD-1 antibody.

The term “Programmed Death-1”, “Programmed cell death protein 1” or “PD-1” is used herein interchangeably and refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863 or Uniprot No. Q15116.

The term “human antibody” (HuMAb) or “fully human antibody” as used herein refers to an Ab having variable regions in which both the framework and CDR regions are derived from human germline immunoglobulin sequences. Furthermore, if the Ab contains a constant region, the constant region also is derived from human germline immunoglobulin sequences. The human Abs of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include Abs in which CDR sequences derived from the germline of another mammalian species, such as a mouse, have been grafted onto human framework sequences. The terms “human” Abs and “fully human” Abs and are used synonymously.

The term “humanized antibody” as used herein refers to an Ab in which some, most or all of the amino acids outside the CDR domains of a non-human Ab are replaced with corresponding amino acids derived from human immunoglobulins. In one embodiment of a humanized form of an Ab, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the Ab to bind to a particular antigen. A “humanized” Ab retains an antigenic specificity similar to that of the original Ab.

The term “chimeric antibody” as used herein refers to an Ab in which the variable regions are derived from one species and the constant regions are derived from another species, such as an Ab in which the variable regions are derived from a mouse Ab and the constant regions are derived from a human Ab.

The term “pharmaceutically acceptable diluents, excipients or carriers” as used herein refers to diluents, excipients or carriers that are suitable for use with humans and/or animals without undue adverse side effects (such as toxicity, irritation, and allergic response) commensurate with a reasonable benefit/risk ratio. “Diluents” are agents which are added to the bulk volume of the active agent making up the solid composition. As a result, the size of the solid composition increases, which makes it easier to handle. Diluents are convenient when the dose of drug per solid composition is low and the solid composition would otherwise be too small. “Excipients” can be binders, lubricants, glidants, coating additives or combinations thereof. Thus, excipients are intended to serve multiple purposes. “Carriers” can be solvents, suspending agents or vehicles, for delivering the instant compounds to a subject.

An “individual,” “subject” or “patient” as used herein is a vertebrate. In certain embodiments, the vertebrate is a mammal. Mammals include, but are not limited to, primates (including human and non-human primates) and rodents (e.g., mice and rats). In preferred embodiments, a subject is a human.

Thus, in a first aspect the present invention provides a pharmaceutical combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM), wherein the ICM is ezabenlimab; and optionally
(c) one or more pharmaceutically acceptable diluents, excipients or carriers.

### Recombinant Gram-Negative Bacterial Strain

In one embodiment the recombinant Gram-negative bacterial strain of the present invention is a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter, and wherein the heterologous protein or a fragment thereof is selected from the group consisting of proteins involved in induction or regulation of an interferon (IFN) response, proteins involved in apoptosis or apoptosis regulation, cell cycle regulators, ankyrin repeat proteins, cell signaling proteins, reporter proteins, transcription factors, proteases, small GTPases, GPCR related proteins, nanobody fusion constructs and nanobodies, bacterial T3SS effectors, bacterial T4SS effectors and viral proteins, or a fragment thereof.

In one embodiment the recombinant Gram-negative bacterial strain of the present invention is a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter, and wherein the heterologous protein or a fragment thereof is a protein involved in induction or regulation of an interferon (IFN) response or a fragment thereof.

In one embodiment the recombinant Gram-negative bacterial strain of the present invention is a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter, and wherein the heterologous protein or a fragment thereof is a protein involved in induction or regulation of a type I IFN response or a fragment thereof.

Preferably the heterologous protein or a fragment thereof is a protein involved in induction or regulation of a type I IFN response or a fragment thereof selected from the group consisting of the RIG-I-like receptor (RLR) family or a fragment thereof, other CARD domain containing proteins involved in antiviral signaling and type I IFN induction or a fragment thereof, and cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, leading to stimulation of STING, or a fragment thereof.

More preferably the heterologous protein or a fragment thereof is a protein involved in induction or regulation of a type I IFN response or a fragment thereof selected from the group consisting of RIG1, MDA5, LGP2, MAVS, WspR, DncV, DisA and DisA-like, CdaA, CdaS and cGAS, or a fragment thereof, more preferably selected from the group consisting of RIG1, MAVS, MDA5, WspR, DncV, DisA-like, and cGAS, or a fragment thereof, most preferably selected from the group consisting of RIG1 or a fragment thereof and cGAS or a fragment thereof, in particular a fragment of RIG1 comprising a CARD domain thereof, more particular a fragment of RIG1 comprising a CARD domain thereof, even more particular a fragment of RIG1, preferably human RIG1, comprising two CARD domains most particular a fragment of human RIG1 comprising two CARD domains as shown in SEQ ID NO: 1, SEQ ID NO: 2 or SEQ ID NO: 3, preferably as shown in SEQ ID NO: 1 and/or cGAS or a fragment thereof e.g. fragments thereof as described supra, in particular the human cGAS or a fragment thereof as shown in SEQ ID NO: 10.

The polynucleotide molecule comprised by the Gram-negative bacterial strain of the present invention can be a vector like an expression vector. The vector comprising the polynucleotide molecule can be a low, medium or high copy number plasmid. Low copy number plasmids have usually 1-15 copies/bacterial cell, preferably 1-10 copies/bacterial cell. Medium copy number plasmids have usually 5-200 copies/bacterial cell, preferably 10-150 copies/bacterial cell. High copy number plasmids have usually 100-1′000 copies/bacterial cell, preferably 150-700 copies/bacterial cell. In a preferred embodiment the vector comprising the polynucleotide molecule is a medium copy number plasmid. In a preferred embodiment the vector is a medium copy number plasmid with 5-200 copies/bacterial cell, i.e. 5-200 copies of the plasmid are present in a single bacterial cell, preferably 10-150 copies/bacterial cell i.e. 10-150 copies of the plasmid are present in a single bacterial cell.

In one embodiment the vector comprising the polynucleotide molecule is a plasmid which has without insert a size of between 1 and 15 kDa, preferably between 2 and 10 kDa, more preferably between 3 and 7 kDa.

In one embodiment the recombinant Gram-negative bacterial strain is a recombinant virulence attenuated Gram-negative bacterial strain.

In one embodiment of the present invention the recombinant Gram-negative bacterial strain is selected from the group consisting of the genera Yersinia, Escherichia, Salmonella and Pseudomonas. In one embodiment the recombinant Gram-negative bacterial strain is selected from the group consisting of the genera Yersinia and Salmonella. Preferably the recombinant Gram-negative bacterial strain is a Yersinia strain, more preferably a Yersinia enterocolitica strain. Most preferred is Yersinia enterocolitica E40 (O:9, biotype 2) (Sory and Cornelis, 1994) or Ampicilline sensitive derivates thereof as Y. enterocolitica MRS40 (also named Y. enterocolitica subsp. palearctica MRS40) as described in (Sarker et al., 1998). Y. enterocolitica E40 and its derivate Y. enterocolitica MRS40 as described in (Sarker et al., 1998) is identical to Y. enterocolitica subsp. palearctica E40 and its derivate Y. enterocolitica subsp. palearctica MRS40 as described in (Howard et al., 2006; Neubauer et al., 2000; Pelludat et al., 2002). Also preferably the recombinant Gram-negative bacterial strain is a Salmonella strain, more preferably a Salmonella enterica strain. Most preferred is Salmonella enterica Serovar Typhimurium SL1344 as described by the Public health England culture collection (NCTC 13347).

In some embodiments of the present invention the recombinant Gram-negative bacterial strain is a strain which does not produce a siderophore e.g. is deficient in the production of a siderophore, preferably does not produce siderophores e.g. is deficient in the production of any siderophore. Such a strain is for example Y. enterocolitica subsp. palearctica MRS40 as described in (Howard et al., 2006; Neubauer et al., 2000; Pelludat et al., 2002; Sarker et al., 1998) which does not produce yersiniabactin and which is preferred.

In one embodiment of the present invention the delivery signal from a bacterial effector protein comprises a bacterial effector protein or a N-terminal fragment thereof, preferably a bacterial effector protein which is virulent toward eukaryotic cells or a N-terminal fragment thereof.

In one embodiment of the present invention the delivery signal from a bacterial effector protein is a bacterial T3SS effector protein comprising a bacterial T3SS effector protein or a N-terminal fragment thereof wherein the T3SS effector protein or a N-terminal fragment thereof may comprise a chaperone binding site. A T3SS effector protein or a N-terminal fragment thereof which comprises a chaperone binding site is particular useful as delivery signal in the present invention. Preferred T3SS effector proteins or N-terminal fragments thereof are selected from the group consisting of SopE, SopE2, SptP, YopE, ExoS, SipA, SipB, SipD, SopA, SopB, SopD, IpgB1, IpgD, SipC, SifA, SseJ, Sse, SrfH, YopJ, AvrA, AvrBsT, YopT, YopH, YpkA, Tir, EspF, TccP2, IpgB2, OspF, Map, OspG, OspI, IpaH, SspH1, VopF, ExoS, ExoT, HopAB2, XopD, AvrRpt2, HopAO1, HopPtoD2, HopU1, GALA family of proteins, AvrBs2, AvrD1, AvrBS3, YopO, YopP, YopE, YopM, YopT, EspG, EspH, EspZ, IpaA, IpaB, IpaC, VirA, IcsB, OspC1, OspE2, IpaH9.8, IpaH7.8, AvrB, AvrD, AvrPphB, AvrPphC, AvrPphEPto, AvrPpiBPto, AvrPto, AvrPtoB, VirPphA, AvrRpm1, HopPtoE, HopPtoF, HopPtoN, PopB, PopP2, AvrBs3, XopD, and AvrXv3. More preferred T3SS effector proteins or N-terminal fragments thereof are selected from the group consisting of SopE, SptP, YopE, ExoS, SopB, IpgB1, IpgD, YopJ, YopH, EspF, OspF, ExoS, YopO, YopP, YopE, YopM, YopT, whereof most preferred T3SS effector proteins or N-terminal fragments thereof are selected from the group consisting of IpgB1, SopE, SopB, SptP, OspF, IpgD, YopH, YopO, YopP, YopE, YopM, YopT, in particular YopE or an N-terminal fragment thereof.

Equally preferred T3SS effector proteins or N-terminal fragments thereof are selected from the group consisting of SopE, SopE2, SptP, SteA, SipA, SipB, SipD, SopA, SopB, SopD, IpgB1, IpgD, SipC, SifA, SifB, SseJ, Sse, SrfH, YopJ, AvrA, AvrBsT, YopH, YpkA, Tir, EspF, TccP2, IpgB2, OspF, Map, OspG, OspI, IpaH, VopF, ExoS, ExoT, HopAB2, AvrRpt2, HopAO1, HopU1, GALA family of proteins, AvrBs2, AvrD1, YopO, YopP, YopE, YopT, EspG, EspH, EspZ, IpaA, IpaB, IpaC, VirA, IcsB, OspC1, OspE2, IpaH9.8, IpaH7.8, AvrB, AvrD, AvrPphB, AvrPphC, AvrPphEPto, AvrPpiBPto, AvrPto, AvrPtoB, VirPphA, AvrRpm1, HopPtoD2, HopPtoE, HopPtoF, HopPtoN, PopB, PopP2, AvrBs3, XopD, and AvrXv3. Equally more preferred T3SS effector proteins or N-terminal fragments thereof are selected from the group consisting of SopE, SptP, SteA, SifB, SopB, IpgB1, IpgD, YopJ, YopH, EspF, OspF, ExoS, YopO, YopP, YopE, YopT, whereof equally most preferred T3SS effector proteins or N-terminal fragments thereof are selected from the group consisting of IpgB1, SopE, SopB, SptP, SteA, SifB, OspF, IpgD, YopH, YopO, YopP, YopE, and YopT, in particular SopE, SteA, or YopE or an N-terminal fragment thereof, more particular SteA or YopE or an N-terminal fragment thereof, most particular YopE or an N-terminal fragment thereof.

In some embodiments the delivery signal from a bacterial effector protein is encoded by a nucleotide sequence comprising the bacterial effector protein or an N-terminal fragment thereof, wherein the N-terminal fragment thereof includes at least the first 10, preferably at least the first 20, more preferably at least the first 100 amino acids of the bacterial T3SS effector protein.

In some embodiments the delivery signal from the bacterial effector protein is encoded by a nucleotide sequence comprising the bacterial T3SS effector protein or an N-terminal fragment thereof, wherein the bacterial T3SS effector protein or the N-terminal fragment thereof comprises a chaperone binding site.

Preferred T3SS effector proteins or a N-terminal fragment thereof, which comprise a chaperone binding site comprise the following combinations of chaperone binding site and T3SS effector protein or N-terminal fragment thereof: SycE-YopE, InvB-SopE, SicP-SptP, SycT-YopT, SycO-YopO, SycN/YscB-YopN, SycH-YopH, SpcS-ExoS, CesF-EspF, SycD-YopB, SycD-YopD. More preferred are SycE-YopE, InvB-SopE, SycT-YopT, SycO-YopO, SycN/YscB-YopN, SycH-YopH, SpcS-ExoS, CesF-EspF. Most preferred is a YopE or an N-terminal fragment thereof comprising the SycE chaperone binding site such as an N-terminal fragment of a YopE effector protein containing the N-terminal 138 amino acids of the YopE effector protein designated herein as YopE1-138 and as shown in SEQ ID NO: 25 or a SopE effector protein or an N-terminal fragment thereof comprising the InvB chaperone binding site such as an N-terminal fragment of a SopE effector protein containing the N-terminal 81 or 105 amino acids of the SopE effector protein designated herein as SopE1-81 or SopE1-105 respectively, and as shown in SEQ ID NOs: 26 and 27.

In one embodiment of the present invention the recombinant Gram-negative bacterial strain is a Yersinia strain and the delivery signal from the bacterial effector protein comprises a YopE effector protein or an N-terminal part, preferably the Y. enterocolitica YopE effector protein or an N-terminal part thereof. Preferably the SycE binding site is comprised within the N-terminal part of the YopE effector protein. In this connection an N-terminal fragment of a YopE effector protein may comprise the N-terminal 12, 16, 18, 52, 53, 80 or 138 amino acids (Feldman et al., 2002; Ittig et al., 2015; Ramamurthi and Schneewind, 2005; Wolke et al., 2011). Most preferred is an N-terminal fragment of a YopE effector protein containing the N-terminal 138 amino acids of the YopE effector protein e.g. as described in Forsberg and Wolf-Watz (Forsberg and Wolf-Watz, 1990) designated herein as YopE1-138 and as shown in SEQ ID NO: 25.

In one embodiment of the present invention the recombinant Gram-negative bacterial strain is a Salmonella strain and the delivery signal from the bacterial effector protein encoded by a nucleotide sequence comprises a SopE or SteA effector protein or an N-terminal part thereof, preferably the Salmonella enterica SopE or SteA effector protein or an N-terminal part thereof. Preferably the chaperon binding site is comprised within the N-terminal part of the SopE effector protein. In this connection an N-terminal fragment of a SopE effector protein protein may comprise the N-terminal 81 or 105 amino acids. Most preferred is the full length SteA (SEQ ID NO: 28) and an N-terminal fragment of a SopE effector protein containing the N-terminal 105 amino acids of the effector protein e.g. as described in SEQ ID NO: 27.

One skilled in the art is familiar with methods for identifying the polypeptide sequences of an effector protein that are capable of delivering a protein. For example, one such method is described by Sory et al. (Sory and Cornelis, 1994). Briefly, polypeptide sequences from e.g. various portions of the Yop proteins can be fused in-frame to a reporter enzyme such as the calmodulin-activated adenylate cyclase domain (or Cya) of the Bordetella pertussis cyclolysin. Delivery of a Yop-Cya hybrid protein into the cytosol of eukaryotic cells is indicated by the appearance of cyclase activity in the infected eukaryotic cells that leads to the accumulation of cAMP. By employing such an approach, one skilled in the art can determine, if desired, the minimal sequence requirement, i.e., a contiguous amino acid sequence of the shortest length, that is capable of delivering a protein, see, e.g. (Sory and Cornelis, 1994). Accordingly, preferred delivery signals of the present invention consists of at least the minimal sequence of amino acids of a T3SS effector protein that is capable of delivering a protein.

In one embodiment, the recombinant Gram-negative bacterial strain is deficient in producing at least one bacterial effector protein, more preferably is deficient in producing at least one bacterial effector protein which is virulent toward eukaryotic cells, even more preferably is deficient in producing at least one T3SS effector protein, most preferably is deficient in producing at least one T3SS effector protein which is virulent toward eukaryotic cells. In some embodiments the recombinant Gram-negative bacterial strains are deficient in producing at least one, preferably at least two, more preferably at least three, even more preferably at least four, in particular at least five, more particular at least six, most particular all bacterial effector proteins which are virulent toward eukaryotic cells. In some embodiments the recombinant Gram-negative bacterial strains are deficient in producing at least one preferably at least two, more preferably at least three, even more preferably at least four, in particular at least five, more particular at least six, most particular all functional bacterial effector proteins which are virulent toward eukaryotic cells such that the resulting recombinant Gram-negative bacterial strain produces less bacterial effector proteins or produces bacterial effector proteins to a lesser extent compared to the non virulence attenuated Gram-negative bacterial wild type strain i.e. compared to the Gram-negative bacterial wild type strain which normally produces bacterial effector proteins or such that the resulting recombinant Gram-negative bacterial strain no longer produce any functional bacterial effector proteins which are virulent toward eukaryotic cells.

According to the present invention, such a mutant Gram-negative bacterial strain i.e. such a recombinant Gram-negative bacterial strain which is deficient in producing at least one bacterial effector protein e.g. which is deficient in producing at least one bacterial effector protein which is virulent toward eukaryotic cells e.g. such a mutant Yersinia strain can be generated by introducing at least one mutation into at least one effector-encoding gene. Preferably, such effector-encoding genes include YopE, YopH, YopO/YpkA, YopM, YopP/YopJ and YopT as far as a Yersinia strain is concerned. Preferably, such effector-encoding genes include AvrA, CigR, GogB, GtgA, GtgE, PipB, SifB, SipA/SspA, SipB, SipC/SspC, SipD/SspD, SirP, SopB/SigD, SopA, SpiC/SsaB, SseB, SseC, SseD, SseF, SseG, SseI/SrfH, SopD, SopE, SopE2, SspH1, SspH2, PipB2, SifA, SopD2, SseJ, SseK1, SseK2, SseK3, SseL, SteC, SteA, SteB, SteD, SteE, SpvB, SpvC, SpvD, SrfJ, SptP, as far as a Salmonella strain is concerned. Most preferably, all effector-encoding genes are deleted. The skilled artisan may employ any number of standard techniques to generate mutations in these T3SS effector genes. Sambrook et al. describe in general such techniques. See Sambrook et al. (Sambrook, 2001).

In accordance with the present invention, the mutation can be generated in the promoter region of an effector-encoding gene so that the expression of such effector gene is abolished. The mutation can also be generated in the coding region of an effector-encoding gene such that the catalytic activity of the encoded effector protein is abolished. The “catalytic activity” of an effector protein refers normally to the anti-target cell function of an effector protein, i.e., toxicity. Such activity is governed by the catalytic motifs in the catalytic domain of an effector protein. The approaches for identifying the catalytic domain and/or the catalytic motifs of an effector protein are well known by those skilled in the art. See, for example, (Alto and Dixon, 2008; Alto et al., 2006).

Accordingly, one preferred mutation of the present invention is a deletion of the entire catalytic domain. Another preferred mutation is a frameshift mutation in an effector-encoding gene such that the catalytic domain is not present in the protein product expressed from such “frameshifted” gene. A most preferred mutation is a mutation with the deletion of the entire coding region of the effector protein. Other mutations are also contemplated by the present invention, such as small deletions or base pair substitutions, which are generated in the catalytic motifs of an effector protein leading to destruction of the catalytic activity of a given effector protein.

The mutations that are generated in the genes of the functional bacterial effector proteins may be introduced into the particular strain by a number of methods. One such method involves cloning a mutated gene into a “suicide” vector which is capable of introducing the mutated sequence into the strain via allelic exchange. An example of such a “suicide” vector is described by (Kaniga et al., 1991).

In this manner, mutations generated in multiple genes may be introduced successively into a Gram-negative bacterial strain giving rise to polymutant, e.g. a sixtuple mutant recombinant strain. The order in which these mutated sequences are introduced is not important. Under some circumstances, it may be desired to mutate only some but not all of the effector genes. Accordingly, the present invention further contemplates polymutant Yersinia other than sixtuple-mutant Yersinia, e.g., double-mutant, triple-mutant, quadruple-mutant and quintuple-mutant strains. For the purpose of delivering proteins, the secretion and translocation system of the instant mutant strain needs to be intact.

A preferred recombinant Gram-negative bacterial strain of the present invention is a sixtuple-mutant Yersinia strain in which all the effector-encoding genes (which are yopH, yopO), yopP, yopE, yopM, yop7) are mutated such that the resulting Yersinia no longer produce any functional effector proteins. Such sixtuple-mutant Yersinia strain is designated as ΔyopH,O,P,E,M,T for Y. enterocolitica. As an example such a sixtuple-mutant can be produced from the Y. enterocolitica MRS40 strain giving rise to Y. enterocolitica MRS40 ΔyopH,O,P,E,M,T (also named Y. enterocolitica subsp. palearctica MRS40 ΔyopH,O,P,E,M,T or Y. enterocolitica ΔyopH,O,P,E,M,T or Y. enterocolitica ΔyopHOPEMT or Y. enterocolitica ΔH,O,P,E,M,T or Y. enterocolitica ΔHOPEMT or Y. enterocolitica MRS40 ΔyopHOPEMT, Y. enterocolitica MRS40 ΔH,O,P,E,M,T or Y. enterocolitica MRS40 ΔHOPEMT or Y. enterocolitica subsp. palearctica MRS40 ΔyopHOPEMT or Y. enterocolitica subsp. palearctica MRS40 ΔH,O,P,E,M,T, or Y. enterocolitica subsp. palearctica MRS40 ΔHOPEMT herein) which is preferred. Y. enterocolitica MRS40 ΔyopH,O,P,E,M,T which is deficient in the production of Yersiniabactin has been described in WO02077249 and was deposited on 24 Sep. 2001, according to the Budapest Treaty on the International Recognition of the Deposit of Microorganisms for the Purposes of Patent Procedure with the Belgian Coordinated Collections of Microorganisms (BCCM) and was given accession number LMG P-21013.

Equally preferred is Y. enterocolitica MRS40 ΔyopH,O,P,E,M,T which comprises a deletion on the endogenous virulence plasmid pYV which removes a RNA hairpin structure or parts thereof such as a deletion of Hairpin I upstream of the gene coding for an endogenous AraC-type DNA binding protein (ΔHairpinI-virF) such as Y. enterocolitica MRS40 ΔyopH,O,P,E,M,T ΔHairpinI-virF (also named Y. enterocolitica ΔyopH,O,P,E,M,T ΔHairpinI-virF). Equally preferred is Y. enterocolitica MRS40 ΔyopH,O,P,E,M,T which comprises a deletion of a chromosomal gene coding for asd and the endogenous virulence plasmid pYV which comprises a nucleotide sequence comprising a gene coding for asd operably linked to a promoter (pYV-asd) such as Y. enterocolitica MRS40 ΔyopH,O,P,E,M,T Δasd pYV-asd (also named Y. enterocolitica ΔyopH,O,P,E,M,T Δasd pYV-asd herein). Particular preferred is Y. enterocolitica MRS40 ΔyopH,O,P,E,M,T Δasd ΔHairpinI-virF pYV-asd which comprises both modifications as described above (also named Y. enterocolitica ΔyopH,O,P,E,M,T Δasd ΔHairpinI-virF pYV-asd herein). Particular preferred strains are Y. enterocolitica MRS40 ΔyopH,O,P,E,M,T ΔHairpinI-virF (also named Y. enterocolitica ΔyopH,O,P,E,M,T ΔHairpinI-virF), Y. enterocolitica MRS40 ΔyopH,O,P,E,M,T Δasd pYV-asd (also named Y. enterocolitica ΔyopH,O,P,E,M,T Δasd pYV-asd herein) or Y. enterocolitica MRS40 ΔyopH,O,P,E,M,T Δasd ΔHairpinI-virF pYV-asd (also named Y. enterocolitica ΔyopH,O,P,E,M,T Δasd ΔHairpinI-virF pYV-asd herein) which are deficient in the production of a siderophore, preferably does not produce siderophores e.g. are deficient in the production of any siderophore, as is the case for all Y. enterocolitica subsp. palearctica strains. Thus, equally particular preferred strains are Y. enterocolitica subsp. palearctica ΔyopH,O,P,E,M,T ΔHairpinI-virF (also named Y. enterocolitica subsp. palearctica ΔyopH,O,P,E,M,T ΔHairpinI-virF), Y. enterocolitica subsp. palearctica ΔyopH,O,P,E,M,T Δasd pYV-asd also named Y. enterocolitica ΔyopH,O,P,E,M,T Δasd pYV-asd herein) or Y. enterocolitica subsp. palearctica ΔyopH,O,P,E,M,T Δasd ΔHairpinI-virF pYV-asd (also named Y. enterocolitica ΔyopH,O,P,E,M,T Δasd ΔHairpinI-virF pYV-asd herein). Most preferred is the sixtuple-mutant Yersinia enterocolitica strain which is designated as ΔyopH,O,P,E,M,T.

Polynucleic acid constructs like vectors which can be used according to the invention to transform a Gram-negative bacterial strain may depend on the Gram-negative bacterial strains used as known to the skilled person. Polynucleic acid constructs which can be used according to the invention include expression vectors (including synthetic or otherwise generated modified versions of endogenous virulence plasmids), vectors for chromosomal or virulence plasmid insertion and nucleotide sequences such as e.g. DNA fragments for chromosomal or virulence plasmid insertion. Expression vectors which are useful in e.g. Yersinia, Escherichia, Salmonella or Pseudomonas strain are e.g. pUC, pBad, pACYC, pUCP20 and pET plasmids. Vectors for chromosomal or virulence plasmid insertion which are useful in e.g. Yersinia, Escherichia, Salmonella or Pseudomonas strain are e.g. pKNG101. DNA fragments for chromosomal or virulence plasmid insertion refer to methods used in e.g. Yersinia, Escherichia, Salmonella or Pseudomonas strain as e.g. lambda-red genetic engineering.

Vectors for chromosomal or virulence plasmid insertion or DNA fragments for chromosomal or virulence plasmid insertion may insert the nucleotide sequences of the present invention so that e.g. the nucleotide sequence encoding a heterologous protein fused in frame to the 3′end of a nucleotide sequence encoding a delivery signal from a bacterial effector protein is operably linked to an endogenous promoter of the recombinant Gram-negative bacterial strain. Thus if a vector for chromosomal or virulence plasmid insertion or a DNA fragment for chromosomal or virulence plasmid insertion is used, an endogenous promoter can be encoded on the endogenous bacterial DNA (chromosomal or plasmid DNA) and only the respective nucleotide sequence will be provided by the engineered vector for chromosomal or virulence plasmid insertion or DNA fragment for chromosomal or virulence plasmid insertion.

Alternatively, if a vector for chromosomal or virulence plasmid insertion or a polynucleic acid construct such as e.g. a nucleotide sequence for chromosomal or virulence plasmid insertion is used, an endogenous promoter and the delivery signal from a bacterial effector protein can be encoded on the endogenous bacterial DNA (chromosomal or plasmid DNA) and only the polynucleic acid construct such as e.g. a nucleotide sequence encoding the heterologous protein will be provided by a vector for chromosomal or virulence plasmid insertion or by a polynucleic acid construct such as e.g. a nucleotide sequence for chromosomal or virulence plasmid insertion. Thus a promoter is not necessarily needed to be comprised by the vector used for transformation of the recombinant Gram-negative bacterial strains i.e. the recombinant Gram-negative bacterial strains of the present invention may be transformed with a vector which does not comprise a promoter.

A preferred vector e.g. a preferred expression vector for Yersinia is selected from the group consisting of pBad_Si_1, pBad_Si_2 and pT3P-715, pT3P-716 and pT3P-717. pBad_Si2 was constructed by cloning of the SycE-YopE₁₋₁₃₈ fragment containing endogenous promoters for YopE and SycE from purified pYV40 into KpnI/HindIII site of pBad-MycHisA (Invitrogen). Additional modifications include removal of the NcoI/BglII fragment of pBad-MycHisA by digest, Klenow fragment treatment and religation. Further at the 3′ end of YopE1-138 the following cleavage sites were added: XbaI-XhoI-BstBI-(HindIII). pBad_Si1 is equal to pBad_Si2 but encodes EGFP amplified from pEGFP-C1 (Clontech) in the NcoI/BglII site under the Arabinose inducible promoter. Equally preferred is the use of modified versions of the endogenous Yersinia virulence plasmid pYV encoding heterologous proteins as fusions to a T3SS signal sequence.

A preferred vector e.g. a preferred expression vector for Salmonella is selected from the group consisting of pT3P_267, pT3P_268 and pT3P_269. Plasmids pT3P_267, pT3P_268 and pT3P_269 containing the corresponding endogenous promoter and the full length SteA sequence (pT3P_267), the SopE1-81 fragment (pT3P_268) or the SopE1-105 fragment (pT3P_269) were amplified from S. enterica SL1344 genomic DNA and cloned into NcoI/KpnI site of pBad-MycHisA (Invitrogen).

pT3P-715 is a fully synthetic plasmid (de-novo synthesized vector) with similar characteristics to pSi_2, while the corresponding AraC coding region has been deleted, and the Ampicillin resistance gene (plus 70 bp upstream) is replaced by a chloramphenicol resistance gene with 200 bp upstream region. For clarity, pT3P-715 comprises the SycE-YopE1-138 fragment containing endogenous promoters for YopE and SycE from pYV40, where at the 3′ end of YopE1-138 the following cleavage sites were added: XbaI-XhoI-BstBI-HindIII. It features a pBR322 origin of replication, and a chloramphenicol acetyl transferase (cat) from transposable genetic element Tn9 (Alton and Vapnek, 1979).

pBad_Si2 and pT3P-715 are medium copy number plasmids with a pBR322 (pMB1) origin of replication (SEQ ID NO: 29).

Derivative pT3P-716 is a high copy number plasmid based on a point mutation in the pBR322 origin of replication (SEQ ID NO: 29), which then results in the ColE1 origin of replication (SEQ ID NO: 30). High-copy number plasmids for expression and delivery of heterologous cargo proteins are based on pT3P-716.

Derivative pT3P-717 is a low copy number plasmid based on the pBR322 origin of replication as in pT3P-715, but additionally comprising the rop (for “repressor of primer”) gene (SEQ ID NO: 31). Low-copy number plasmids for expression and delivery of heterologous cargo proteins are based on pT3P-717.

The polynucleotide molecules of the instant invention may include other sequence elements such as a 3′ termination sequence (including a stop codon and a poly A sequence), or a gene conferring a drug resistance which allows the selection of transformants having received the polynucleotide molecules or other element allowing selection of transformants.

The polynucleotide molecules of the present invention may be transformed by a number of known methods into the recombinant Gram-negative bacterial strains. For the purpose of the present invention, the methods of transformation for introducing the polynucleotide molecules include, but are not limited to, electroporation, calcium phosphate mediated transformation, conjugation, or combinations thereof. For example, a polynucleotide molecules e.g. located on a vector can be transformed into a first bacteria strain by a standard electroporation procedure. Subsequently, such a polynucleotide molecule e.g. located on a vector can be transferred from the first bacteria strain into the desired strain by conjugation, a process also called “mobilization”. Transformant (i.e., Gram-negative bacterial strains having taken up the vector) may be selected, e.g., with antibiotics. These techniques are well known in the art. See, for example, (Sory and Cornelis, 1994).

In accordance with the present invention, the promoter operably linked to the bacterial effector protein of the recombinant Gram-negative bacterial strain of the invention can be a native promoter of a T3SS effector protein of the respective strain or a compatible bacterial strain, or another native promoter of the respective or a compatible bacterial strain or a promoter used in expression vectors which are useful in e.g. Yersinia, Escherichia, Salmonella or Pseudomonas strain e.g pUC and pBad. Such promoters are the T7 promoter, Plac promoter or the arabinose inducible Ara-bad promoter.

If the recombinant Gram-negative bacterial strain is a Yersinia strain the promoter can be from a Yersinia virulon gene. A “Yersinia virulon gene” refers to genes on the Yersinia pYV plasmid, the expression of which is controlled both by temperature and by contact with a target cell. Such genes include genes coding for elements of the secretion machinery (the Ysc genes), genes coding for translocators (YopB, YopD, and LcrV), genes coding for the control elements (YopN, TyeA and LcrG), genes coding for T3SS effector chaperones (SycD, SycE, SycH, SycN, SycO and SycT), and genes coding for effectors (YopE, YopH, YopO/YpkA, YopM, YopT and YopP/YopJ) as well as other pYV encoded proteins as VirF and YadA.

In a preferred embodiment of the present invention, the promoter is the native promoter of a T3SS functional effector encoding gene. If the recombinant Gram-negative bacterial strain is a Yersinia strain the promoter is selected from any one of YopE, YopH, YopO/YpkA, YopM and YopP/YopJ. More preferably, the promoter is from YopE and/or YopH. Most preferred is the YopE and the YopH promoter, respectively.

If the recombinant Gram-negative bacterial strain is a Salmonella strain the promoter can be from SpiI or SpiII pathogenicity island or from an effector protein elsewhere encoded. Such genes include genes coding for elements of the secretion machinery, genes coding for translocators, genes coding for the control elements, genes coding for T3SS effector chaperones, and genes coding for effectors as well as other proteins encoded by SPI-1 or SPI-2. In a preferred embodiment of the present invention, the promoter is the native promoter of a T3SS functional effector encoding gene. If the recombinant Gram-negative bacterial strain is a Salmonella strain the promoter is selected from any one of the effector proteins. More preferably, the promoter is from SopE, InvB or SteA.

In some embodiments the promoter is an artificially inducible promoter, as e.g. the IPTG inducible promoter, a light inducible promoter and the arabinose inducible promoter.

In one embodiment of the present invention the recombinant Gram-negative bacterial strain comprises a nucleotide sequence encoding a protease cleavage site. The protease cleavage site is usually located on the polynucleotide molecule comprising a nucleotide sequence encoding a heterologous protein fused in frame to the 3′end of a nucleotide sequence encoding a delivery signal from a bacterial effector protein between the nucleotide sequence encoding a heterologous protein and the nucleotide sequence encoding a delivery signal. Generation of a functional and generally applicable cleavage site allows cleaving off the delivery signal after translocation. As the delivery signal can interfere with correct localization and/or function of the translocated protein within the target cells the introduction of a protease cleavage site between the delivery signal and the protein of interest provides delivery of almost native proteins into eukaryotic cells. Preferably the protease cleavage site is an amino acid motif which is cleaved by a protease or the catalytic domains thereof selected from the group consisting of enterokinase (light chain), enteropeptidase, prescission protease, human rhinovirus protease 3C, TEV protease, TVMV protease, FactorXa protease and thrombin, more preferably an amino acid motif which is cleaved by TEV protease. Equally preferable the protease cleavage site is an amino acid motif which is cleaved by a protease or the catalytic domains thereof selected from the group consisting of enterokinase (light chain), enteropeptidase, prescission protease, human rhinovirus protease 3C, TEV protease, TVMV protease, FactorXa protease, ubiquitin processing protease, called Deubiquitinating enzymes, and thrombin. Most preferred is an amino acid motif which is cleaved by TEV protease or by an ubiquitin processing protease.

Thus in a further embodiment of the present invention, the heterologous protein is cleaved from the delivery signal from a bacterial effector protein by a protease. Preferred methods of cleavage are methods wherein:
a) the protease is translocated into the eukaryotic cell by a recombinant Gram-negative bacterial strain as described herein which expresses a fusion protein which comprises the delivery signal from the bacterial effector protein and the protease as heterologous protein; or
b) the protease is expressed constitutively or transiently in the eukaryotic cell.

Usually the recombinant Gram-negative bacterial strain used to deliver a desired protein into a eukaryotic cell and the recombinant Gram-negative bacterial strain translocating the protease into the eukaryotic cell are different.

In one embodiment of the present invention the recombinant Gram-negative bacterial strain comprises a further nucleotide sequence encoding a labelling molecule or an acceptor site for a labelling molecule. The further nucleotide sequence encoding a labelling molecule or an acceptor site for a labelling molecule is usually fused to the 5′ end or to the 3′ end of the nucleotide sequence encoding a heterologous protein. A preferred labelling molecule or an acceptor site for a labelling molecule is selected from the group consisting of enhanced green fluorescent protein (EGFP), coumarin, coumarin ligase acceptor site, resorufin, resurofin ligase acceptor site, the tetra-Cysteine motif in use with FLASH/ReAsH dye (life technologies). Most preferred is resorufin and a resurofin ligase acceptor site or EGFP. The use of a labelling molecule or an acceptor site for a labelling molecule will lead to the attachment of a labelling molecule to the heterologous protein of interest, which will then be delivered as such into the eukaryotic cell and enables tracking of the protein by e.g. live cell microscopy.

In one embodiment of the present invention the recombinant Gram-negative bacterial strain comprises a further nucleotide sequence encoding a peptide tag. The further nucleotide sequence encoding a peptide tag is usually fused to the 5′ end or to the 3′ end of the nucleotide sequence encoding a heterologous protein. A preferred peptide tag is selected from the group consisting of Myc-tag, His-tag, Flag-tag, HA tag, Strep tag or V5 tag or a combination of two or more tags out of these groups. Most preferred is Myc-tag, Flag-tag, His-tag and combined Myc- and His-tags. The use of a peptide tag will lead to traceability of the tagged protein e.g by immunofluorescence or Western blotting using anti-tag antibodies. Further, the use of a peptide tag allows affinity purification of the desired protein either after secretion into the culture supernatant or after translocation into eukaryotic cells, in both cases using a purification method suiting the corresponding tag (e.g. metal-chelate affinity purification in use with a His-tag or anti-Flag antibody based purification in use with the Flag-tag).

In one embodiment of the present invention the recombinant Gram-negative bacterial strain comprises a further nucleotide sequence encoding a nuclear localization signal (NLS). The further nucleotide sequence encoding a nuclear localization signal (NLS) is usually fused to the 5′end or to the 3′end of the nucleotide sequence encoding a heterologous protein wherein said further nucleotide sequence encodes a nuclear localization signal (NLS). A preferred NLS is selected from the group consisting of SV40 large T-antigen NLS and derivates thereof (Yoneda et al., 1992) as well as other viral NLS. Most preferred is SV40 large T-antigen NLS and derivates thereof.

In one embodiment of the present invention the recombinant Gram-negative bacterial strain comprises a multiple cloning site. The multiple cloning site is usually located at the 3′end of the nucleotide sequence encoding a delivery signal from a bacterial effector protein and/or at the 5′end or 3′end of the nucleotide sequence encoding a heterologous protein. One or more than one multiple cloning sites can be comprised by the vector. A preferred multiple cloning site is selected from the group of restriction enzymes consisting of XhoI, XbaI, HindIII, NcoI, NotI, EcoRI, EcoRV, BamHI, NheI, SacI, SalI, BstBI. Most preferred is XbaI, XhoI, BstBI and HindIII.

The protein expressed by the recombinant Gram-negative bacterial strain of the present invention is also termed as a “fusion protein” or a “hybrid protein”, i.e., is a fused protein or hybrid of delivery signal and a heterologous protein. The fusion protein can also comprise e.g. a delivery signal and two or more different heterologous proteins. In some embodiments at least two fusion proteins which comprise each a delivery signal from a bacterial effector protein and a heterologous protein are expressed by the recombinant Gram-negative bacterial strain and are translocated into the eukaryotic cell e.g. the cancer cell by the methods of the present inventions.

The recombinant Gram-negative bacterial strain can be cultured so that a fusion protein is expressed which comprises the delivery signal from the bacterial effector protein and the heterologous protein according to methods known in the art (e.g. FDA, Bacteriological Analytical Manual (BAM), chapter 8: Yersinia enterocolitica). Preferably the recombinant Gram-negative bacterial strain can be cultured in Brain Heart infusion broth e.g. at 28° C. For induction of expression of T3SS and e.g. YopE/SycE promoter dependent genes, bacteria can be grown at 37° C.

Those skilled in the art can also use a number of assays to determine whether the delivery of a heterologous protein like a fusion protein by the recombinant Gram-negative bacteria is successful. For example, the fusion protein may be detected via immunofluorescence using antibodies recognizing a fused tag (like Myc-tag). The determination can also be based on the enzymatic activity of the protein being delivered, e.g., the assay described by (Sory and Cornelis, 1994).

In a preferred embodiment the recombinant Gram-negative bacterial strain of the present invention is a recombinant Gram-negative bacterial strain which comprises
i) a first polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
ii) a second polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
iii) a third polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter; and
iv) a fourth polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter,
wherein said first and said second polynucleotide molecule are located on a vector comprised by said Gram-negative bacterial strain and said third and said fourth polynucleotide molecule are located on a chromosome of said Gram-negative bacterial strain or on an extra-chromosomal genetic element comprised by said Gram-negative bacterial strain, with the proviso that the extra-chromosomal genetic element is not the vector on which the said first and said second polynucleotide molecule are located.

In one embodiment the third and fourth polynucleotide molecule is located on an endogenous virulence plasmid, preferably located on an endogenous virulence plasmid at the native site of a bacterial effector protein e.g. at the native site of a virulence factor, preferably in case the recombinant Gram-negative bacterial strain is a Yersinia strain, at the native site of YopE and/or YopH or at the native site of another Yop (YopO, YopP, YopM, YopT), preferably at the native site of YopE and YopH, respectively, or in case the recombinant Gram-negative bacterial strain is a Salmonella strain at the native site of an effector protein encoded within SpiI, SpiII or encoded elsewhere, preferably at the native site of an effector protein encoded within SpiI or SpiII, more preferably at the native site of SopE or SteA.

In one embodiment the nucleotide sequence encoding a heterologous protein or a fragment thereof of the first polynucleotide molecule and the nucleotide sequence encoding a heterologous protein or a fragment thereof of the third polynucleotide molecule encode the same heterologous protein or a fragment thereof.

In a further embodiment the nucleotide sequence encoding a heterologous protein or a fragment thereof of the second polynucleotide molecule and the nucleotide sequence encoding a heterologous protein or a fragment thereof of the fourth polynucleotide molecule encode the same heterologous protein or a fragment thereof.

In a preferred embodiment the nucleotide sequence encoding a heterologous protein or a fragment thereof of the first polynucleotide molecule and the nucleotide sequence encoding a heterologous protein or a fragment thereof of the third polynucleotide molecule encode the same heterologous protein or a fragment thereof and the nucleotide sequence encoding a heterologous protein or a fragment thereof of the second polynucleotide molecule and the nucleotide sequence encoding a heterologous protein or a fragment thereof of the fourth polynucleotide molecule encode the same heterologous protein or a fragment thereof, wherein the heterologous protein or a fragment thereof encoded by the first and third polynucleotide molecule is different from the heterologous protein or a fragment thereof encoded by the second and fourth polynucleotide molecule.

In a more preferred embodiment the heterologous protein or a fragment thereof encoded by the nucleotide sequence of the first and the third polynucleotide molecule, independently of each other, is selected from the group consisting of the RIG-I-like receptor (RLR) family or a fragment thereof, other CARD domain containing proteins involved in antiviral signaling and type I IFN induction or a fragment thereof, and cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, leading to stimulation of STING, or a fragment thereof as described supra. Even more preferably the heterologous protein encoded by the nucleotide sequence of the first and the third polynucleotide molecule, independently of each other, is selected from the group consisting of RIG1, MDA5, MAVS, WspR, DncV, DisA and DisA-like, CdaA, and cGAS, or a fragment thereof as described supra. In particular the heterologous protein encoded by the nucleotide sequence of the first and the third polynucleotide molecule is cGAS or a fragment thereof e.g. fragments thereof as described supra, more particular the human cGAS or a fragment thereof as shown in SEQ ID NO: 10.

In a more preferred embodiment the heterologous protein or a fragment thereof encoded by the nucleotide sequence of the second and the fourth polynucleotide molecule, independently of each other, is selected from the group consisting of the RIG-I-like receptor (RLR) family or a fragment thereof, other CARD domain containing proteins involved in antiviral signaling and type I IFN induction or a fragment thereof, and cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, leading to stimulation of STING, or a fragment thereof as described supra. Even more preferably the heterologous protein encoded by the nucleotide sequence of the second and the fourth polynucleotide molecule, independently of each other, is selected from the group consisting of RIG1, MDA5, MAVS, WspR, DncV, DisA and DisA-like, CdaA, and cGAS, or a fragment thereof. In particular the heterologous protein encoded by the nucleotide sequence of the second and the fourth polynucleotide molecule, independently of each other, is selected from the group consisting of RIG1, MDA5, MAVS, WspR, DncV, DisA and DisA-like, and CdaA, or a fragment thereof. More particular the heterologous protein encoded by the nucleotide sequence of the second and the fourth polynucleotide molecule is RIG1 or a fragment thereof as described supra, more particular a fragment of RIG1 comprising a CARD domain, even more particular a fragment of RIG1, preferably human RIG1, comprising two CARD domains most particular a fragment of human RIG1 comprising two CARD domains as shown in SEQ ID NO: 1, SEQ ID NO: 2 or SEQ ID NO: 3, preferably as shown in SEQ ID NO: 1.

In some embodiments the delivery signal from a bacterial effector protein of the first, second, third and fourth polynucleotide molecule is the same delivery signal. In a preferred embodiment the delivery signal from a bacterial effector protein of the first, second, third and fourth polynucleotide molecule is a delivery signal from a bacterial T3SS effector protein, preferably the same delivery signal from a bacterial T3SS effector protein. In a more preferred embodiment the delivery signal from a bacterial effector protein of the first, second, third and fourth polynucleotide molecule comprises the YopE effector protein or an N-terminal fragment thereof.

In one embodiment the nucleotide sequence encoding a heterologous protein or a fragment thereof fused in frame to the 3′end of the nucleotide sequence encoding a delivery signal from a bacterial effector protein of the first polynucleotide molecule and the nucleotide sequence encoding a heterologous protein or a fragment thereof fused in frame to the 3′end of the nucleotide sequence encoding a delivery signal from a bacterial effector protein of the second polynucleotide molecule are each operably linked to the same promoter. The term “each operably linked to the same promoter” means in this connection that one promoter (the same promoter) drives expression of the heterologous proteins of the first and the second polynucleotide molecule. In a preferred embodiment the nucleotide sequence encoding a heterologous protein or a fragment thereof fused in frame to the 3′end of the nucleotide sequence encoding a delivery signal from a bacterial effector protein of the first polynucleotide molecule and the nucleotide sequence encoding a heterologous protein or a fragment thereof fused in frame to the 3′end of the nucleotide sequence encoding a delivery signal from a bacterial effector protein of the second polynucleotide molecule are each operably linked to the same YopE promoter.

In a further embodiment the nucleotide sequence encoding a heterologous protein or a fragment thereof fused in frame to the 3′end of the nucleotide sequence encoding a delivery signal from a bacterial effector protein of the third polynucleotide molecule and the nucleotide sequence encoding a heterologous protein or a fragment thereof fused in frame to the 3′end of the nucleotide sequence encoding a delivery signal from a bacterial effector protein of the fourth polynucleotide molecule are operably linked to two different promoters. In a preferred embodiment the nucleotide sequence encoding a heterologous protein or a fragment thereof fused in frame to the 3′end of the nucleotide sequence encoding a delivery signal from a bacterial effector protein of the third polynucleotide molecule is operably linked to the YopE promoter and the nucleotide sequence encoding a heterologous protein or a fragment thereof fused in frame to the 3′end of the nucleotide sequence encoding a delivery signal from a bacterial effector protein of the fourth polynucleotide molecule is operably linked to the YopH promoter.

In a further preferred embodiment the nucleotide sequence encoding a heterologous protein or a fragment thereof fused in frame to the 3′end of the nucleotide sequence encoding a delivery signal from a bacterial effector protein of the first polynucleotide molecule and the nucleotide sequence encoding a heterologous protein or a fragment thereof fused in frame to the 3′end of the nucleotide sequence encoding a delivery signal from a bacterial effector protein of the second polynucleotide molecule are operably linked to the same promoter and the nucleotide sequence encoding a heterologous protein or a fragment thereof fused in frame to the 3′end of the nucleotide sequence encoding a delivery signal from a bacterial effector protein of the third polynucleotide molecule and the nucleotide sequence encoding a heterologous protein or a fragment thereof fused in frame to the 3′end of the nucleotide sequence encoding a delivery signal from a bacterial effector protein of the fourth polynucleotide molecule are operably linked to two different promoters.

The vector comprising said first and second polynucleotide molecule can be a low, medium or high copy number plasmid. Low copy number plasmids have usually 1-15 copies/bacterial cell, preferably 1-10 copies/bacterial cell. Medium copy number plasmids have usually 5-200 copies/bacterial cell, preferably 10-150 copies/bacterial cell. High copy number plasmids have usually 100-1′000 copies/bacterial cell, preferably 150-700 copies/bacterial cell.

In a preferred embodiment the vector comprising said first and second polynucleotide molecule is a medium copy number plasmid. In a preferred embodiment the vector is a medium copy number plasmid with 5-200 copies/bacterial cell, i.e. 5-200 copies of the plasmid are present in a single bacterial cell, preferably 10-150 copies/bacterial cell i.e. 10-150 copies of the plasmid are present in a single bacterial cell.

In one embodiment the vector comprising said first and second polynucleotide molecule is a plasmid which has without insert a size of between 1 and 15 kDa, preferably between 2 and 10 kDa, more preferably between 3 and 7 kDa.

In one embodiment the extra-chromosomal genetic element is an endogenous virulence plasmid, preferably an endogenous virulence plasmid which naturally (in nature) encodes proteins of the type III secretion system. In a preferred embodiment the extra-chromosomal genetic element is the endogenous virulence plasmid pYV.

The recombinant Gram-negative bacterial strain of the present invention can be obtained by
1) transforming a Gram-negative bacterial strain with a polynucleotide molecule, preferably a DNA polynucleotide molecule, comprising a nucleotide sequence encoding a heterologous protein and a nucleotide sequence which is homologous or identical to a nucleotide sequence encoding a delivery signal from a bacterial effector protein or which is homologous or identical to a nucleotide sequence encoding a fragment of a delivery signal from a bacterial effector protein, wherein the delivery signal from a bacterial effector protein or a fragment thereof is encoded on the chromosome or on an endogenous virulence plasmid of the Gram-negative bacterial strain. Preferably the nucleotide sequence which is homologous or identical to a nucleotide sequence of a delivery signal from a bacterial effector protein or to a fragment thereof is located on the 5′ end of the nucleotide sequence encoding a heterologous protein. The nucleotide sequence encoding a heterologous protein can be flanked on its 3′ end by a nucleotide sequence homologous to the nucleotide sequence of the chromosome or of the endogenous virulence plasmid at the 3′ end of the delivery signal from a bacterial effector protein or to a fragment thereof. This nucleotide sequence flanking the homologous protein on its 3′ end can be homologous to the nucleotide sequence lying within 10 kbp on the chromosome or on an endogenous virulence plasmid at the 3′ end of the delivery signal from a bacterial effector protein or to a fragment thereof. This nucleotide sequence flanking the homologous protein on its 3′ end can be homologous to the nucleotide sequence and can be within the same operon on the chromosome or on an endogenous virulence plasmid as the delivery signal from a bacterial effector protein or a fragment thereof. Transformation is usually performed so that the nucleotide sequence encoding a heterologous protein is inserted on an endogenous virulence plasmid or a chromosome of the recombinant Gram-negative bacterial strain, preferably on an endogenous virulence plasmid, at the 3′end of a delivery signal from a bacterial effector protein encoded by the chromosome or the endogenous virulence plasmid, wherein the heterologous protein fused to the delivery signal is expressed and secreted;
2) Subsequently (or in parallel) to step 1), the recombinant bacterial strain can be transformed with a further polynucleotide molecule, preferably a DNA polynucleotide molecule, comprising a nucleotide sequence encoding a heterologous protein and a nucleotide sequence which is homologous or identical to a nucleotide sequence encoding a delivery signal from a bacterial effector protein or which is homologous or identical to a nucleotide sequence encoding a fragment of a delivery signal from a bacterial effector protein, wherein the delivery signal from a bacterial effector protein or a fragment thereof is encoded on the chromosome or on an endogenous virulence plasmid of the Gram-negative bacterial strain. The nucleotide sequence can be homologous or identical to a nucleotide sequence of a delivery signal from a bacterial effector protein or to a fragment thereof can be located on the 5′ end of the nucleotide sequence encoding a heterologous protein. The nucleotide sequence encoding a heterologous protein can be flanked on its 3′ end by a nucleotide sequence homologous to the nucleotide sequence of the chromosome or of the endogenous virulence plasmid at the 3′ end of the delivery signal from a bacterial effector protein or to a fragment thereof. This nucleotide sequence flanking the homologous protein on its 3′ end can be homologous to the nucleotide sequence lying within 10 kbp on the chromosome or on an endogenous virulence plasmid at the 3′ end of the delivery signal from a bacterial effector protein or to a fragment thereof. This nucleotide sequence flanking the homologous protein on its 3′ end can be homologous to the nucleotide sequence and can be within the same operon on the chromosome or on an endogenous virulence plasmid as the delivery signal from a bacterial effector protein or a fragment thereof. Transformation is usually performed so that the nucleotide sequence encoding a heterologous protein is inserted on an endogenous virulence plasmid or a chromosome of the recombinant Gram-negative bacterial strain, preferably on an endogenous virulence plasmid, at the 3′end of a delivery signal from a bacterial effector protein encoded by the chromosome or the endogenous virulence plasmid, wherein the heterologous protein fused to the delivery signal is expressed and secreted; and/or
3) The recombinant bacterial strain can be genetically transformed with one or two polynucleotide construct(s) like an expression vector comprising one (in case of two vectors) or two (in case of one vector) nucleotide sequence(s) encoding a heterologous protein and a nucleotide sequence which is homologous or identical to a nucleotide sequence encoding a delivery signal from a bacterial effector protein or which is homologous or identical to a nucleotide sequence encoding a fragment of a delivery signal from a bacterial effector protein.

In case the recombinant bacterial strain obtained under 1) and 2) is transformed in step 3) with one vector comprising two nucleotide sequences encoding each a heterologous protein and a nucleotide sequence which is homologous or identical to a nucleotide sequence encoding a delivery signal from a bacterial effector protein or which is homologous or identical to a nucleotide sequence encoding a fragment of a delivery signal from a bacterial effector protein, these two sequences can be fused to form an operon.

Order of steps 1-3) can be interchanged, or steps may be combined or only individual steps may be performed. In case the recombinant Gram-negative bacterial strain is a Yersinia strain the endogenous virulence plasmid is pYV (plasmid of Yersinia Virulence). In case the recombinant Gram-negative bacterial strain is a Salmonella strain, the endogenous location for insertion is one of the gene clusters called SpiI or SpiII (for Salmonella pathogenicity island), a position where an effector protein is elsewhere encoded or alternatively one of the Salmonella virulence plasmids (SVPs).

### Immune Checkpoint Modulators

The ICM used in the present invention is the antagonistic PD-1 antibody ezabenlimab.

### Combinations

A pharmaceutical combination according to the invention is for example a combined preparation or a pharmaceutical composition, for simultaneous, separate or sequential use.

The term “combined preparation” as used herein defines especially a “kit of parts” in the sense that said recombinant Gram-negative bacterial strain and said immune checkpoint modulator can be dosed independently, either in separate form or by use of different fixed combinations with distinguished amounts of the active ingredients. The ratio of the amount of recombinant Gram-negative bacterial strain to the amount of immune checkpoint modulator to be administered in the combined preparation can be varied, e.g. in order to cope with the needs of a patient sub-population to be treated or the needs of a single patient, which needs can be different due to age, sex, body weight, etc. of a patient. The individual parts of the combined preparation (kit of parts) can be administered simultaneously or sequentially, i.e. chronologically staggered, e.g. at different time points and with equal or different time intervals for any part of the kit of parts.

The term “pharmaceutical composition” refers to a fixed-dose combination (FDC) that includes the recombinant Gram-negative bacterial strain and the immune checkpoint modulator combined in a single dosage form, having a predetermined combination of respective dosages.

The pharmaceutical combination further may be used as add-on therapy. As used herein, “add-on” or “add-on therapy” means an assemblage of reagents for use in therapy, the subject receiving the therapy begins a first treatment regimen of one or more reagents prior to beginning a second treatment regimen of one or more different reagents in addition to the first treatment regimen, so that not all of the reagents used in the therapy are started at the same time. For example, adding immune checkpoint modulator therapy to a patient already receiving a Gram-negative bacterial strain therapy.

In a preferred embodiment, the pharmaceutical combination according to the invention is a combined preparation.

The amount of the recombinant Gram-negative bacterial strain and the immune checkpoint modulator to be administered will vary depending upon factors such as the particular compound, disease condition and its severity, according to the particular circumstances surrounding the case, including, e.g., the specific recombinant Gram-negative bacterial strain being administered, the route of administration, the condition being treated, the target area being treated, and the subject or host being treated.

In one embodiment, the invention provides a pharmaceutical combination comprising a recombinant Gram-negative bacterial strain and an immune checkpoint modulator (ICM), wherein said recombinant Gram-negative bacterial strain and said immune checkpoint modulator are present in a therapeutically effective amount.

The expression “effective amount” or “therapeutically effective amount” as used herein refers to an amount capable of invoking one or more of the following effects in a subject receiving the combination of the present invention: (i) inhibition or arrest of tumor growth, including, reducing the rate of tumor growth or causing complete growth arrest; (ii) complete tumour regression; (iii) reduction in tumor size; (iv) reduction in tumor number; (v) inhibition of metastasis (i.e., reduction, slowing down or complete stopping) of tumor cell infiltration into peripheral organs; (vi) enhancement of antitumor immune response, which may, but does not have to, result in the regression or elimination of the tumor; (vii) relief, to some extent, of one or more symptoms associated with cancer; (viii) increase in progression-free survival (PFS) and/or; overall survival (OS) of the subject receiving the combination.

Determination of a therapeutically effective amount is well within the capability of those skilled in the art, especially in light of the detailed disclosure provided herein. In some embodiments, a therapeutically effective amount may (i) reduce the number of cancer cells; (ii) reduce tumor size; (iii) inhibit, retard, slow to some extent, and preferably stop cancer cell infiltration into peripheral organs; (iv) inhibit (e.g., slow to some extent and preferably stop) tumor metastasis; (v) inhibit tumor growth; (vi) delay occurrence and/or recurrence of a tumor; and/or (vii) relieve to some extent one or more of the symptoms associated with the cancer. In various embodiments, the amount is sufficient to ameliorate, palliate, lessen, and/or delay one or more of symptoms of cancer.

In another preferred embodiment, the invention provides a pharmaceutical combination comprising a recombinant Gram-negative bacterial strain and an immune checkpoint modulator (ICM), wherein said recombinant Gram-negative bacterial strain and said immune checkpoint modulator are present in an amount producing an additive therapeutic effect.

As used herein, the term “additive” means that the effect achieved with the pharmaceutical combinations of this invention is approximately the sum of the effects that result from using the anti-cancer agents, namely the recombinant Gram-negative bacterial strain and the immune checkpoint modulator, as a monotherapy.

In another preferred embodiment, the invention provides a pharmaceutical combination comprising a recombinant Gram-negative bacterial strain and an immune checkpoint modulator (ICM), wherein said recombinant Gram-negative bacterial strain and said immune checkpoint modulator are present in an amount producing a synergistic therapeutic effect.

As used herein, the term “synergistic” means that the effect achieved with the pharmaceutical combinations of this invention is greater than the sum of the effects that result from using the anti-cancer agents, namely the recombinant Gram-negative bacterial strain and the immune checkpoint modulator, as a monotherapy. Advantageously, such synergy provides for greater efficacy at the same doses, and may lead to longer duration of response to the therapy.

In one embodiment, the invention provides a pharmaceutical combination comprising an immune checkpoint modulator (ICM) and a recombinant Gram-negative bacterial strain, wherein the amount of said immune checkpoint modulator in the combination is from about 1 to about 1000 mg.

In a preferred embodiment, the invention provides a pharmaceutical combination comprising an immune checkpoint modulator (ICM) and a recombinant Gram-negative bacterial strain, wherein the amount of said inhibitor of an anti-apoptotic protein in the combination is from about 10⁵ to about 10¹⁰ bacteria.

In one embodiment the present invention provides a combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM); and optionally
(c) one or more pharmaceutically acceptable diluents, excipients or carriers;
wherein the recombinant Gram-negative bacterial strain is selected from the group consisting of the genera Yersinia, Escherichia, Salmonella, and Pseudomonas; and
wherein the ICM is the antagonistic PD-1 antibody ezabenlimab.

In one embodiment the present invention provides a combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM); and optionally
(c) one or more pharmaceutically acceptable diluents, excipients or carriers;
wherein the recombinant Gram-negative bacterial strain is selected from the group consisting of the genera Yersinia and Salmonella; and
wherein the ICM is the antagonistic PD-1 antibody ezabenlimab.

In one embodiment the present invention provides a combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM); and optionally
(c) one or more pharmaceutically acceptable diluents, excipients or carriers;
wherein the recombinant Gram-negative bacterial strain is a Yersinia strain; and
wherein the ICM is the antagonistic PD-1 antibody ezabenlimab.

In one embodiment the present invention provides a combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM); and optionally
(c) one or more pharmaceutically acceptable diluents, excipients or carriers;
wherein the recombinant Gram-negative bacterial strain is Yersinia enterocolitica; and
wherein the ICM is the antagonistic PD-1 antibody ezabenlimab.

In one embodiment the present invention provides a combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM); and optionally
(c) one or more pharmaceutically acceptable diluents, excipients or carriers;
wherein the recombinant Gram-negative bacterial strain is Y. enterocolitica MRS40 ΔyopH,O,P,E,M,T; and
wherein the ICM is the antagonistic PD-1 antibody ezabenlimab.

According to the present invention, therapies using the combination of the invention, such as combinations comprising components (a) and (b) as set out above, can be further combined with an analysis of appropriate biomarkers. Biomarkers suitable for the use in connection with ezabenlimab are known in the art. Biomarkers which can be used in the context of the present invention are e.g. biomarkers which can be detected in peripheral blood.

### Formulations, Modes of Administration and Dosing

The formulation and route of administration may be tailored to the individual subject, the nature of the condition to be treated in the subject, and generally, the judgment of the attending practitioner.

The pharmaceutical combination, i.e. the pharmaceutical compositions or combined preparations of the invention may be administered in either single or multiple doses by any of the accepted modes of administration of agents having similar utilities, including rectal, buccal, intranasal, transmucosal, transdermal, by intra-arterial injection, by infusion, intravenously (infusion or injection), intraperitoneally, parenterally, intramuscularly, sub-cutaneous, orally, and topically including intratumoral injection,

A pharmaceutical combination for use in a method for the prevention, delay of progression or treatment of cancer in a subject, as described herein according to the invention is, preferably, suitable for injection, e.g. intravenous, intramuscular, intrathecal, intratumoural or intraperitoneal injection, or infusion, more preferably suitable for intravenous or intratumoural injection or infusion, and usually comprises a therapeutically effective amount of the active ingredients and one or more suitable pharmaceutically acceptable diluent, excipient or carrier.

Thus in a preferred embodiment the pharmaceutical combination is administered to the subject intravenously or intratumorally. i.e. immune checkpoint modulator and recombinant virulence attenuated Gram-negative bacterial strain, are administered to the subject intravenously or intratumoural, in particular by intravenous infusion.

Modes of administration of the immune checkpoint modulator to a subject may be selected from the group consisting of intravenous, intratumoral, sub-cutaneous, intramuscular or intraperitoneal administration. Although this invention is not intended to be limited to any particular mode of application, intravenous or intraperitoneal administration of immune checkpoint modulator is preferred.

The forms in which an immune checkpoint modulator, may be incorporated for administration by injection or infusion include aqueous or oil suspensions, or emulsions, with sesame oil, corn oil, cottonseed oil, or peanut oil, as well as elixirs, mannitol, dextrose, sucrose, or a sterile aqueous solution, and similar pharmaceutical vehicles. Aqueous solutions in saline may also conventionally be used for injection or infusion, preferably physiologically compatible buffers such as Hank's solution, Ringer's solution, L-Histidine buffer, sodium citrate or physiological saline buffer are used as aqueous solutions. Ethanol, glycerol, propylene glycol, liquid polyethylene glycol, (glacial) acetic acid, pentetic acid, and the like (and suitable mixtures thereof), cyclodextrin derivatives, polysorbate and vegetable oils may also be employed. The proper fluidity can be maintained, for example, by the use of a coating, such as lecithin, by the maintenance of the required particle size in the case of dispersion and by the use of surfactants. Injectable solutions are prepared by incorporating a compound according to the present disclosure in the required amount in the appropriate solvent with various other ingredients as enumerated above, as required. Generally, dispersions are prepared by incorporating the various sterilized active ingredients into a sterile vehicle which contains the basic dispersion medium and the required other ingredients from those enumerated above. In the case of powders for the preparation of injectable solutions, the preferred methods of preparation are vacuum-drying and freeze-drying techniques which yield a powder of the active ingredient plus any additional desired ingredient. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For sub-cutaneous delivery of ezabenlimab in the context of the present invention, bioavailability may be improved by the use of an agent which facilitates distribution and absorption of this antibody.

Modes of administration of the recombinant Gram-negative bacteria to a subject may be selected from the group consisting of intravenous, intratumoral, intraperitoneal and per-oral administration. Although this invention is not intended to be limited to any particular mode of application, intravenous or intratumoral administration of the bacteria is preferred.

Pharmaceutical compositions or combined preparations in separate form comprising immune checkpoint modulator and recombinant virulence attenuated Gram-negative bacterial strain, may be manufactured by means of conventional mixing, dissolving, granulating, coated tablet-making, levigating, emulsifying, encapsulating, entrapping or lyophilizing processes. Pharmaceutical compositions or combined preparations in separate form may be formulated in conventional manner using one or more physiologically acceptable carriers, diluents, excipients or auxiliaries which facilitate processing of the active ingredient into preparations which can be used pharmaceutically. Proper formulation depends upon the method of administration chosen.

The unit content of active ingredients in an individual dose need not in itself constitute a therapeutically effective amount, since such an amount can be reached by the administration of a plurality of dosage units. A composition according to the invention may contain, e.g., from about 10% to about 100% of the therapeutically effective amount of the active ingredients.

Where the pharmaceutical combination according to the invention is a combined preparation, said recombinant Gram-negative bacterial strain is preferably not be administered in the same dosage form as said immune checkpoint modulator.

An exemplary treatment regime entails administration once daily, twice daily, three times daily, every second day, twice per week, once per week, once every other week, once every three weeks, once per month, or once every 6 weeks. The combination of the invention is usually administered on multiple occasions. Intervals between single dosages can be, for example, less than a day, daily, every second day, twice per week, weekly, bi-weekly, once every three weeks, once every month, or once every 6 weeks. The combination of the invention may be given as a continuous uninterrupted treatment. The combination of the invention may also be given in a regime in which the subject receives cycles of treatment interrupted by a drug holiday or period of non-treatment. Thus, the combination of the invention may be administered according to the selected intervals above for a continuous period of one week or a part thereof, for two weeks, for three weeks, for four weeks, for five weeks or for six weeks and then stopped for a period of one week, or a part thereof, for two weeks, for three weeks, for four weeks, for five weeks, or for six weeks. The combination of the treatment interval and the non-treatment interval is called a cycle. The cycle may be repeated one or more times. Two or more different cycles may be used in combination for repeating the treatment one or more times. Intervals can also be irregular as indicated by measuring blood (or tumour) levels of said recombinant Gram-negative bacterial strain and/or said immune checkpoint modulator in the patient. In one embodiment, the pharmaceutical combination according to the invention is administered once daily. In an exemplary treatment regime the recombinant Gram-negative bacterial strain can be administered from about 10⁵ to about 10¹⁰ bacteria per day and the immune checkpoint modulator can be administered from 1-1000 mg per day.

Dosis regimens of the administration of the recombinant Gram-negative bacterial strain described herein will vary with the particular goal to be achieved, the age and physical condition of the subject being treated, the duration of treatment, the nature of concurrent therapy and the specific bacterium employed, as known to the skilled person. The recombinant Gram-negative bacterial strain is usually administered to the subject according to a dosing regimen consisting of a single dose every 1-20 days, preferably every 1-10 days, more preferably every 1-7 days. The period of administration is usually about 20 to about 60 days, preferably about 30-40 days. Alternatively the period of administration is usually about 8 to about 32 weeks, preferably about 8 to about 24 weeks, more preferably about 12 to about 16 weeks.

The present invention also provides a pharmaceutical combination comprising a recombinant Gram-negative bacterial strain as described herein optionally comprising a suitable pharmaceutically acceptable diluent, excipient or carrier.

The recombinant Gram-negative bacteria can be compounded for convenient and effective administration in an amount that is sufficient to treat the subject as pharmaceutical composition with a suitable pharmaceutically acceptable carrier. A unit dosage form of the recombinant Gram-negative bacteria or of the pharmaceutical composition to be administered e.g. for a human being of 70 kg, can, for example, contain the recombinant Gram-negative bacteria in an amount from about 10⁵ to about 10¹⁰ bacteria per dosage form, preferably about 10⁶ to about 10⁹ bacteria per dosage form, more preferably about 10⁷ to about 10⁹ bacteria per dosage form, most preferably about 10⁸ bacteria per dosage form; or from about 10⁵ to about 10¹⁰ bacteria per ml, preferably about 10⁶ to about 10⁹ bacteria per ml, more preferably about 10⁷ to about 10⁹ bacteria per ml, most preferably about 10⁸ bacteria per ml.

By “amount that is sufficient to treat the subject” or “effective amount” which are used herein interchangeably is meant to be an amount of a bacterium or bacteria, high enough to significantly positively modify the condition to be treated but low enough to avoid serious side effects (at a reasonable benefit/risk ratio), within the scope of sound medical judgment. An effective amount of a bacterium will vary with the particular goal to be achieved, the age and physical condition of the subject being treated, the duration of treatment, the nature of concurrent therapy and the specific bacterium employed. The effective amount of a bacterium will thus be the minimum amount, which will provide the desired effect. Usually an amount from about 10⁵ to about 10¹⁰ bacteria e.g. from about 10⁵ to about 10¹⁰ bacteria/m² body surface, preferably from about 10⁶ to about 10⁹ bacteria e.g. from about 10⁶ to about 10⁹ bacteria/m² body surface, more preferably from about 10⁷ to about 10⁸ bacteria e.g. from about 10⁷ to about 10⁸ bacteria/m² body surface, most preferably 10⁸ bacteria e.g. 10⁸ bacteria/m² body surface are administered to the subject.

A single dose of the recombinant Gram-negative bacterial strain to administer to a subject, e.g. to a human to treat cancer e.g. a malignant solid tumor is usually from about 10⁴ to about 10¹⁰ bacteria e.g. from about 10⁴ bacteria/m² body surface to about 10¹⁰ bacteria/m² body surface, preferably from about 10⁵ to about 10⁹ bacteria e.g. from about 10⁵ to about 10⁹ bacteria/m² body surface, more preferably from about 10⁶ to about 10⁸ bacteria e.g. from about 10⁶ to about 10⁸ bacteria/m² body surface, even more preferably from about 10⁷ to about 10⁸ bacteria e.g. from about 10⁷ to about 10⁸ bacteria/m² body surface, most preferably 10⁸ bacteria e.g. 10⁸ bacteria/m² body surface of total recombinant Gram-negative bacteria.

Examples of substances which can serve as pharmaceutical carriers are sugars, such as lactose, glucose and sucrose; starches and its derivatives such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; calcium carbonate; vegetable oils, such as peanut oils, cotton seed oil, sesame oil, olive oil, corn oil and oil of theobroma; polyols such as propylene glycol, glycerine, sorbitol, polysorbate, manitol, and polyethylene glycol; agar; alginic acids; pyrogen-free water; isotonic saline; cranberry extracts and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tabletting agents, stabilizers, anti-oxidants and preservatives, can also be present.

A single dose of the immune checkpoint modulator comprises a dosage ranging from 0.01 mg/kg to 100 mg/kg body weight, preferably a dosage from 1 to 20 mg/kg body weight, wherein the typical body weight of a human being is 70 kg.

Depending on the route of administration, the active ingredients which comprise bacteria may be required to be coated in a material to protect said organisms from the action of enzymes, acids and other natural conditions which may inactivate said organisms. In order to administer bacteria by other than parenteral administration, they should be coated by, or administered with, a material to prevent inactivation. For example, bacteria may be co-administered with enzyme inhibitors or in liposomes. Enzyme inhibitors include pancreatic trypsin inhibitor, diisopropylfluorophosphate (DFP) and trasylol. Liposomes include water-in-oil-in-water P40 emulsions as well as conventional and specifically designed liposomes which transport bacteria, such as Lactobacillus, or their by-products to an internal target of a host subject.

One bacterium may be administered alone or in conjunction with a second, different bacterium. Any number of different bacteria may be used in conjunction. By “in conjunction with” is meant together, substantially simultaneously or sequentially. The compositions may be also administered in the form of tablet, pill or capsule, for example, such as a freeze-dried capsule comprising the bacteria of the present invention or as frozen solution of bacteria of the present invention containing DMSO or glycerol. Another preferred form of application involves the preparation of a lyophilized capsule of the bacteria of the present invention. Still another preferred form of application involves the preparation of a heat dried capsule of the bacteria of the present invention. The composition may be formulated and prepared as freeze-dried cake, which is reconstituted in a suitable buffer before administration as liquid form (suspension or solution).

The recombinant Gram-negative bacteria to be administered can be administered by injection. Forms suitable for injectable use include monoseptic suspensions and monoseptic powders for the extemporaneous preparation of monoseptic injectable suspension. In all cases the form must be monoseptic and must be fluid to the extent that easy syringability exists. It must be stable under the conditions of manufacture and storage. The carrier can be a solvent or dispersion medium containing, for example, water, sugars, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents or physiologically compatible buffers, for example, sugars, sodium chloride or L-Histidine buffer. Prolonged absorption of the injectable compositions can be brought about by the use in the compositions of agents delaying absorption, for example, aluminum monostearate and gelatin.

In some embodiments of the present invention the recombinant Gram-negative bacterial strain is co-administered with a siderophore to the subject. These embodiments are preferred. Siderophores which can be co-administered are siderophores including hydroxamate, catecholate and mixed ligand siderophores. Preferred siderophores are Deferoxamine (also known as desferrioxamine B, desferoxamine B, DFO-B, DFOA, DFB or desferal), Desferrioxamine E, Deferasirox (Exjade, Desirox, Defrijet, Desifer) and Deferiprone (Ferriprox), more preferred is Deferoxamine. Deferoxamine is a bacterial siderophore produced by the Actinobacteria Streptomyces pilosus and is commercially available from e.g. Novartis Pharma Schweiz AG (Switzerland).

Co-administration with a siderophore can be before, simultaneous to or after administration of the recombinant Gram-negative bacterial strain. Preferably a siderophore is administered before the administration of recombinant Gram-negative bacterial strain, more preferably is administered at about 24 hours before, preferably about 6 hours before, more preferably 3 hours before, hours, in particular 1 hour before the administration of the recombinant Gram-negative bacterial strain to the subject. In a particular embodiment the subject is pretreated with desferrioxamine 1 hour prior to infection with the recombinant Gram-negative bacterial strain in order to allow bacterial growth. Usually a siderophore is co-administered at a single dose from about 0.5×10⁻⁵ Mol to about 1×10⁻³ Mol, more preferably from about 1×10⁻⁵ Mol to about 5×10⁻⁴ Mol preferably from about 1×10⁻⁴ Mol to about 4×10⁻⁴ Mol. Usually desferoxamine is co-administered at single dose from about 20 mg to about 500 mg preferably from about 50 mg to about 200 mg per subject, more preferably a single dose of 100 mg desferrioxamine is co-administered.

In one embodiment, the cancer cell e.g. the cell of a malignant solid tumor is contacted with two recombinant Gram-negative bacterial strains, wherein the first recombinant Gram-negative bacterial strain expresses a first fusion protein which comprises the delivery signal from the bacterial effector protein and a first heterologous protein and the second recombinant Gram-negative bacterial strain expresses a second fusion protein which comprises the delivery signal from the bacterial effector protein and a second heterologous protein, so that the first and the second fusion protein are translocated into the cell of a malignant solid tumor or a cell of the tumor microenvironment. This embodiment provided for co-infection of a cancer cell e.g. a cell of a malignant solid tumor with two bacterial strains as a valid method to deliver e.g. two different hybrid proteins into single cells.

### Using the Combinations of the Invention to Treat Cancer

According to a second aspect the present invention provides a pharmaceutical combination as described herein, for use as a medicament.

According to a third aspect the present invention provides a pharmaceutical combination as described herein, for use in a method for the prevention, delay of progression or treatment of cancer in a subject.

Also provided is the use of a pharmaceutical combination as described herein for the manufacture of a medicament for the prevention, delay of progression or treatment of cancer in a subject.

Also provided is the use of a pharmaceutical combination as described herein for the prevention, delay of progression or treatment of cancer in a subject.

Also provided is a method for the prevention, delay of progression or treatment of cancer in a subject, comprising administering to said subject a therapeutically effective amount of a pharmaceutical combination as described herein.

The terms “treatment”/“treating” as used herein includes: (1) delaying the appearance of clinical symptoms of the state, disorder or condition developing in an animal, particularly a mammal and especially a human, that may be afflicted with or predisposed to the state, disorder or condition but does not yet experience or display clinical or subclinical symptoms of the state, disorder or condition; (2) inhibiting the state, disorder or condition (e.g. arresting, reducing or delaying the development of the disease, or a relapse thereof in case of maintenance treatment, of at least one clinical or subclinical symptom thereof); and/or (3) relieving the condition (i.e. causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

As used herein, “delay of progression” means increasing the time to appearance of a symptom of a cancer or a mark associated with a cancer or slowing the increase in severity of a symptom of a cancer. Further, “delay of progression” as used herein includes reversing or inhibition of disease progression. “Inhibition” of disease progression or disease complication in a subject means preventing or reducing the disease progression and/or disease complication in the subject.

Preventive treatments comprise prophylactic treatments. In preventive applications, the pharmaceutical combination of the invention is administered to a subject suspected of having, or at risk for developing cancer. In therapeutic applications, the pharmaceutical combination is administered to a subject such as a patient already suffering from cancer, in an amount sufficient to cure or at least partially arrest the symptoms of the disease. Amounts effective for this use will depend on the severity and course of the disease, previous therapy, the subject's health status and response to the drugs, and the judgment of the treating physician. In the case wherein the subject's condition does not improve, the pharmaceutical combination of the invention may be administered chronically, which is, for an extended period of time, including throughout the duration of the subject's life in order to ameliorate or otherwise control or limit the symptoms of the subject's disease or condition.

In the case wherein the subject's status does improve, the pharmaceutical combination may be administered continuously; alternatively, the dose of drugs being administered may be temporarily reduced or temporarily suspended for a certain length of time (i.e., a “drug holiday”). Once improvement of the patient's condition has occurred, a maintenance dose of the pharmaceutical combination of the invention is administered if necessary. Subsequently, the dosage or the frequency of administration, or both, is optionally reduced, as a function of the symptoms, to a level at which the improved disease is retained.

In one embodiment, the cancer is selected from the group consisting of Sarcoma, Leukemia, Lymphoma, multiple myeloma, Central nervous system cancers, and malignant solid tumors, which include, but are not limited to, abnormal mass of cells which may stem from different tissue types such as liver, colon, colorectum, skin, breast, pancreas, cervix uteri, corpus uteri, bladder, gallbladder, kidney, larynx, lip, oral cavity, oesophagus, ovary, prostate, stomach, testis, thyroid gland or lung and thus include malignant solid liver, colon, colorectum, skin, breast, pancreas, cervix uteri, corpus uteri, bladder, gallbladder, kidney, larynx, lip, oral cavity, oesophagus, ovary, prostate, stomach, testis, thyroid gland or lung tumors. Preferably the cancer is a solid tumor, preferably a malignant solid tumor. More preferably the cancer is selected from the group consisting of breast cancer, melanoma and colon cancer (colon carcinoma).

In one embodiment the solid tumor is a malignant solid tumor in a subject, wherein the recombinant Gram-negative bacterial strain accumulates in the malignant solid tumor. Thus in one embodiment the solid tumor is a malignant solid tumor in a subject, wherein the recombinant Gram-negative bacterial strain accumulates in the malignant solid tumor, the method comprising administering to the subject said recombinant Gram-negative bacterial strain, wherein the recombinant Gram-negative bacterial strain is administered in an amount that is sufficient to treat the subject.

The heterologous proteins of the Gram-negative bacterial strain may be delivered i.e. translocated into the cancer cell e.g. to cells of a malignant solid tumor at the time of administering the recombinant Gram-negative bacterial strain to a subject or may be delivered i.e. translocated into the cancer cell e.g. to cells of a malignant solid tumor or cells of the tumor microenvironment at a later time e.g. after the recombinant Gram-negative bacterial strain has reached a cancer cell e.g. the site of the malignant solid tumor and/or has reached a cancer cell e.g. the site of the malignant solid tumor and has replicated as described above. The time of delivery can be regulated e.g. by the promoter used to express the heterologous proteins in the recombinant Gram-negative bacterial strain. In the first case, either a constitutive promoter or, more preferred, an endogenous promoter of a bacterial effector protein might drive the heterologous protein. In the case of delayed protein delivery, an artificially inducible promoter, as the arabinose inducible promoter, might drive the heterologous protein. In this case, arabinose (or an inducer of a corresponding inducible promoter) will be administered to a subject once bacteria have reached and accumulated at the desired site. Arabinose will then induce the bacterial expression of the protein to be delivered.

In one embodiment the method of treating cancer comprises
i) culturing the recombinant Gram-negative bacterial strain as described herein;
ii) administering to the subject said recombinant Gram-negative bacterial strain of i) wherein a fusion protein which comprises a delivery signal from a bacterial effector protein and the heterologous protein is expressed by the recombinant Gram-negative bacterial strain and is translocated into the cancer cell or a cell of the tumor microenvironment; and optionally
iii) cleaving the fusion protein so that the heterologous protein is cleaved from the delivery signal from the bacterial effector protein inside of the cancer cell,
wherein the recombinant Gram-negative bacterial strain is administered in an amount that is sufficient to treat the subject.

The recombinant Gram-negative bacterial strain and the ICM may be administered simultaneously or sequentially, in either order. Hence, the recombinant Gram-negative bacterial strain may be administrated e.g. prior, concurrent or subsequent to the ICM or vice versa. Prior administration as used herein refers to prior initiation of treatment with one compound (e.g. the ICM) before initiation of treatment with the other compound (e.g. the Gram-negative bacterial strain). Subsequent administration as used herein refers to subsequent initiation of treatment with one compound (e.g. the ICM) after the initiation of treatment with the other compound (e.g. the Gram-negative bacterial strain). Concurrent administration as used herein refers to simultaneous initiation of treatment with both compounds (the ICM and the Gram-negative bacterial strain) i.e. on the same day of administration.

### Kit of Parts

The present invention also provides a kit for treating cancer e.g. such as malignant solid tumors, preferably in human. Such kits generally will comprise the pharmaceutical combination as described herein, and instructions for using the kit. In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic. In a further embodiment, the kit comprises bundled containers comprising the medicaments as set out before.

Thus in one embodiment the present invention provides a kit of parts comprising a first container, a second container and a package insert, wherein the first container comprises at least one dose of a medicament comprising a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter, the second container comprises at least one dose of a medicament comprising the immune checkpoint inhibitor (ICM), wherein the ICM is ezabenlimab; and the package insert optionally comprises instructions for treating a subject for cancer using the medicaments.

### EXAMPLES

The present examples are intended to illustrate the present invention without restricting it.

### Example 1

A) Materials and Methods

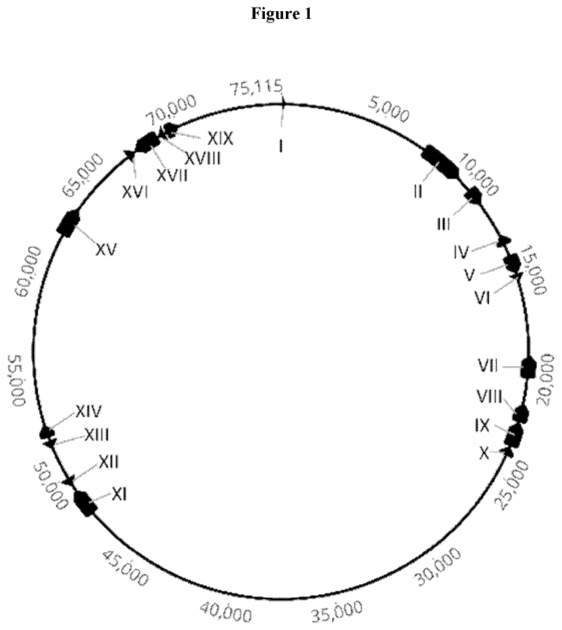

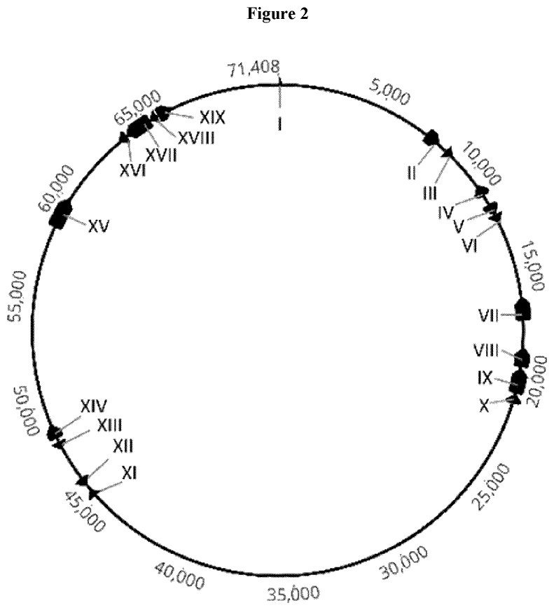

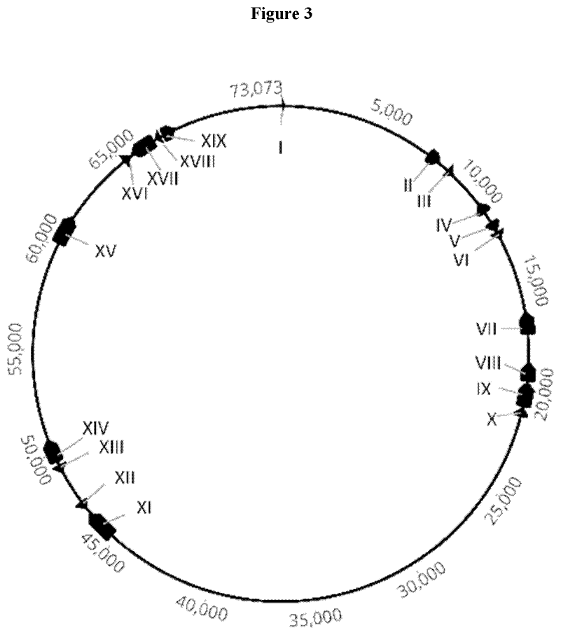

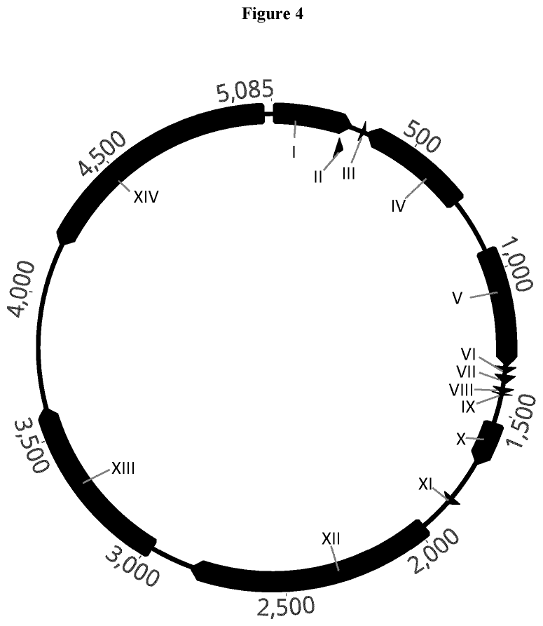

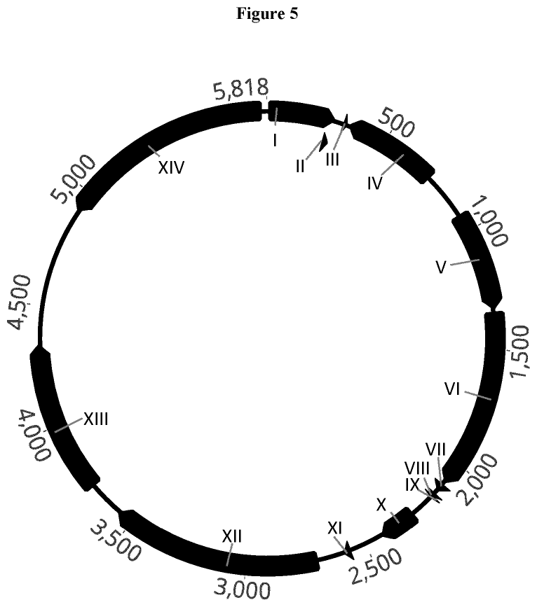

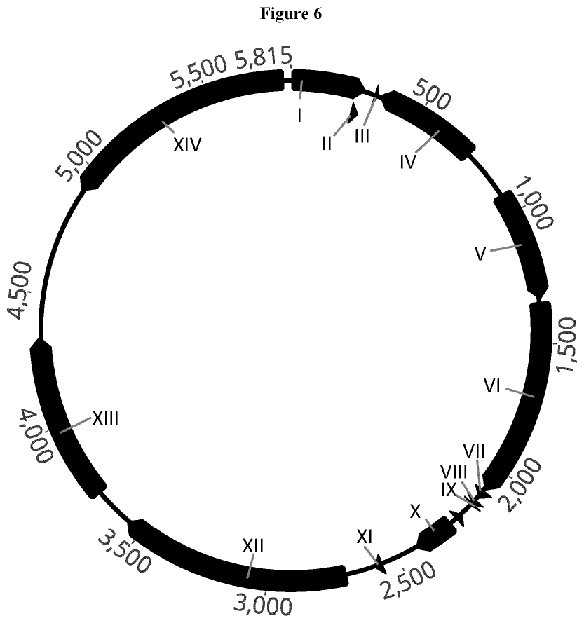

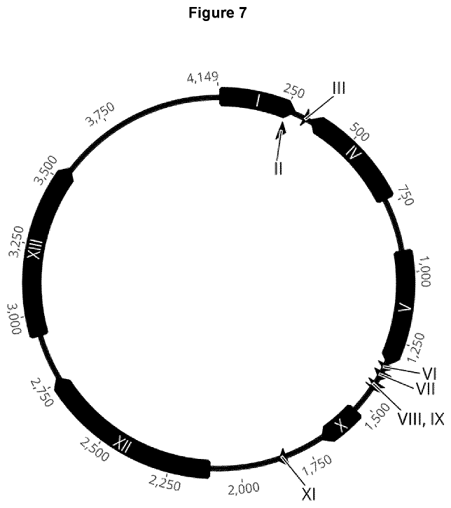

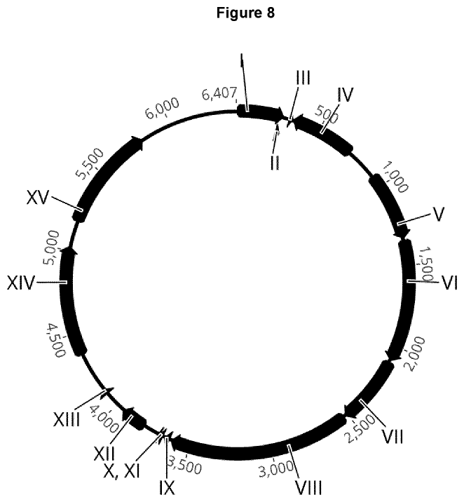

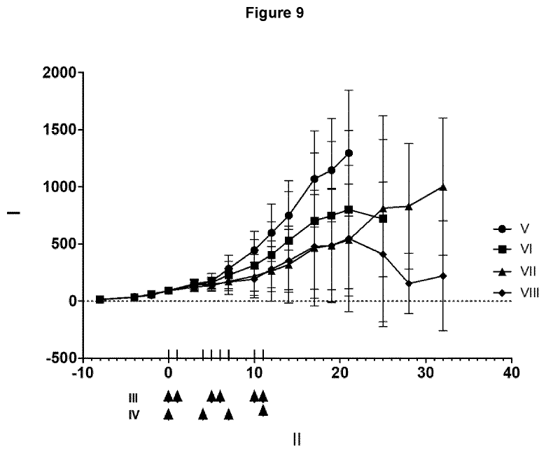

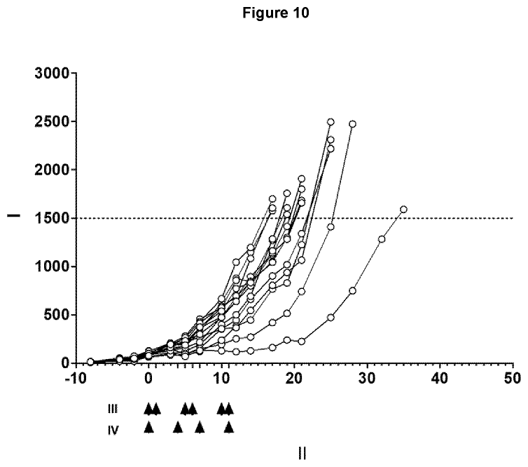

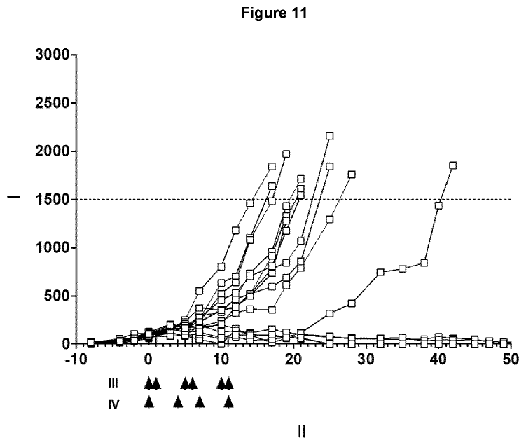

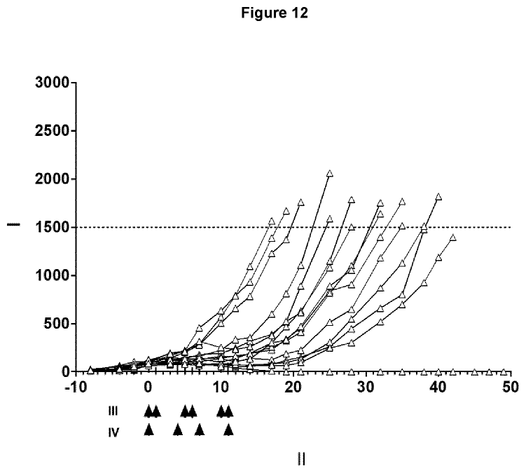

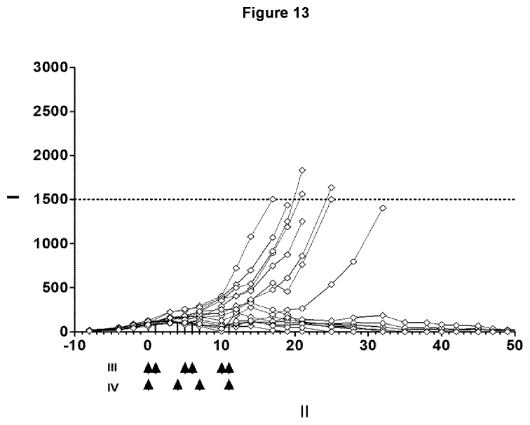

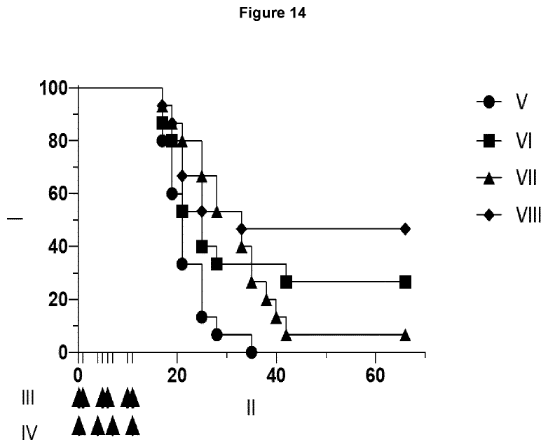

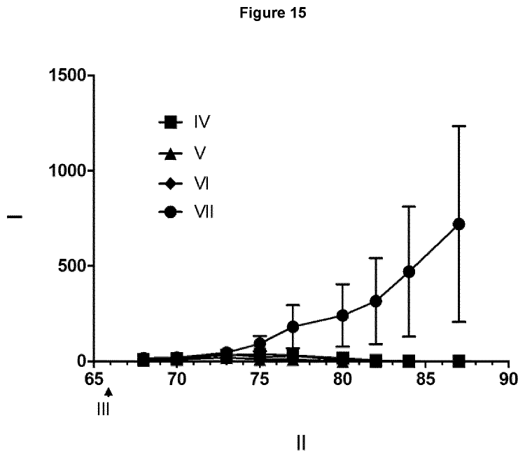

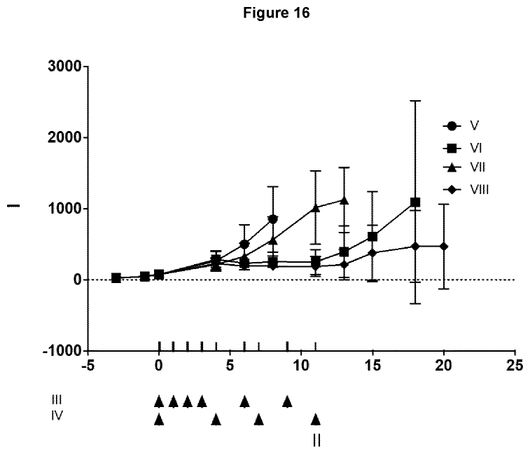

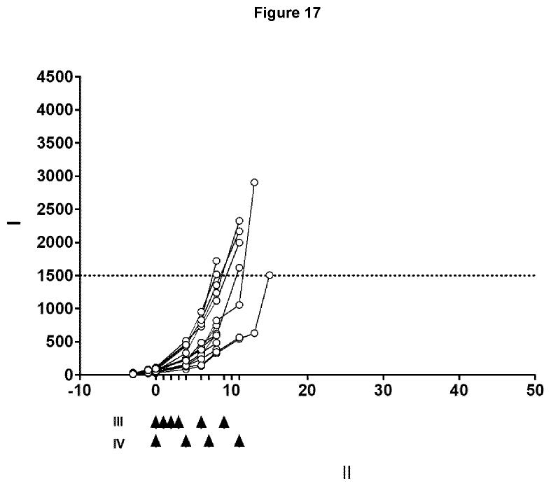

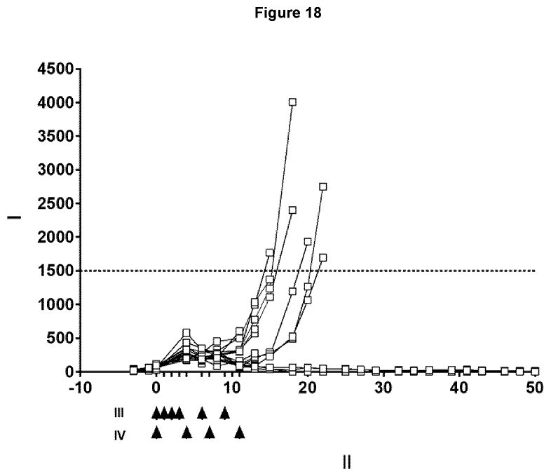

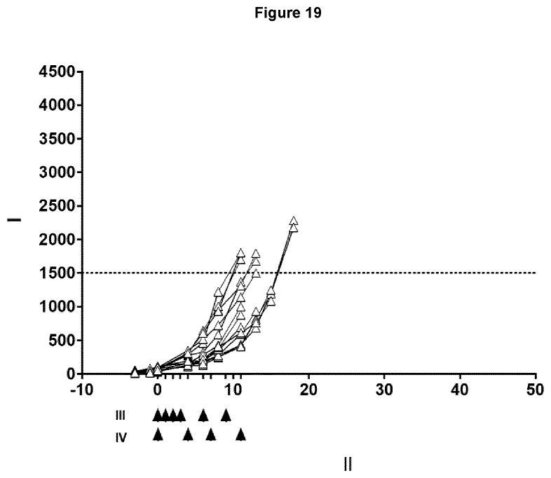

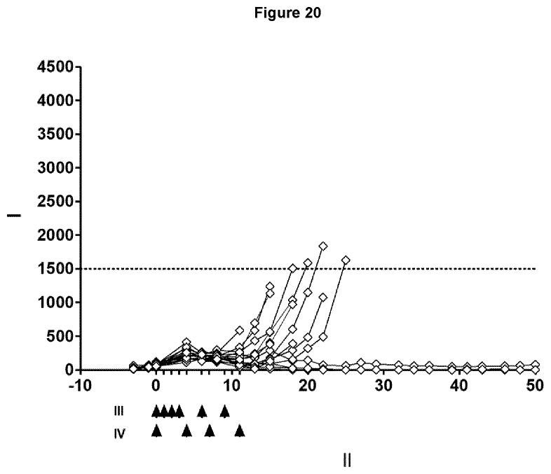

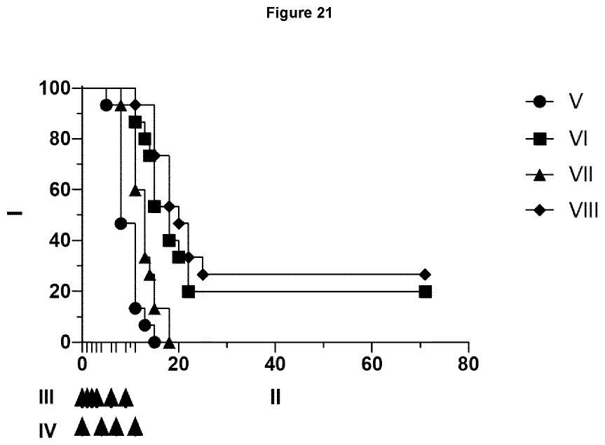

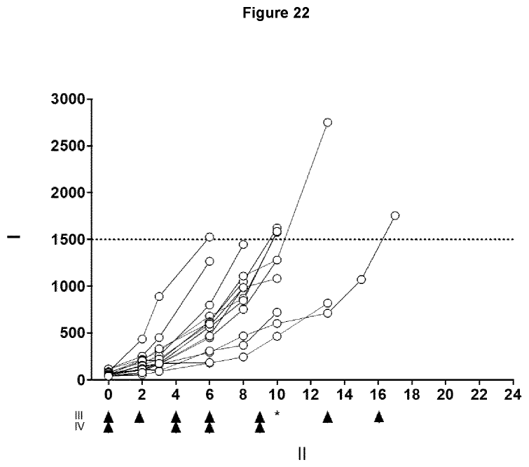

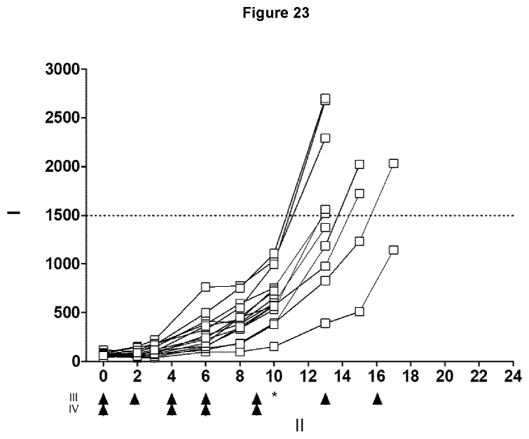

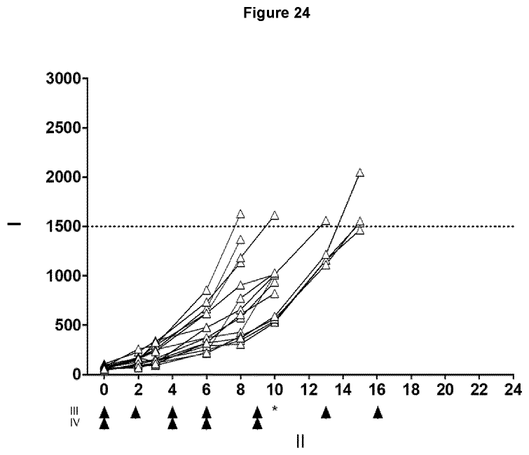

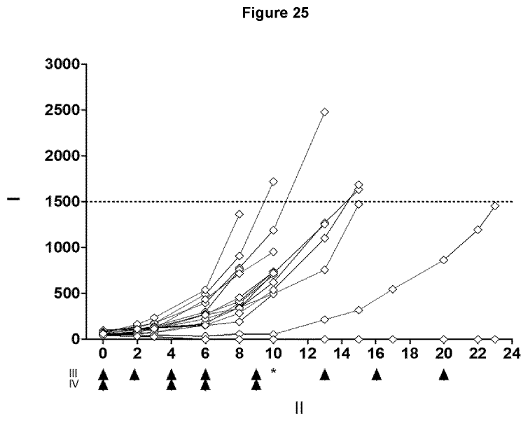

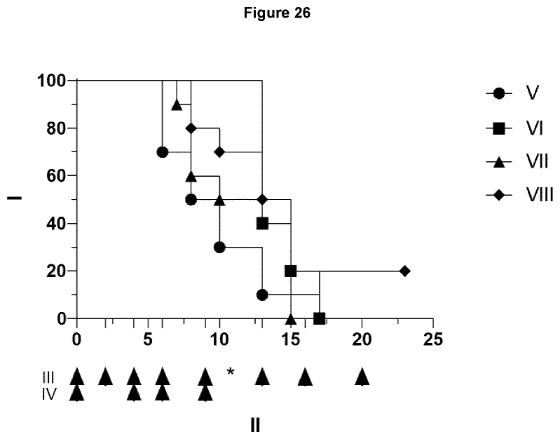

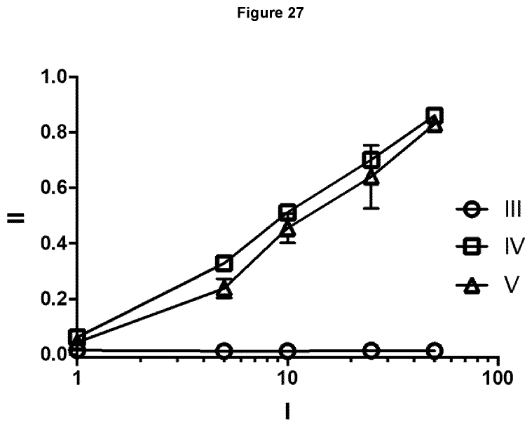

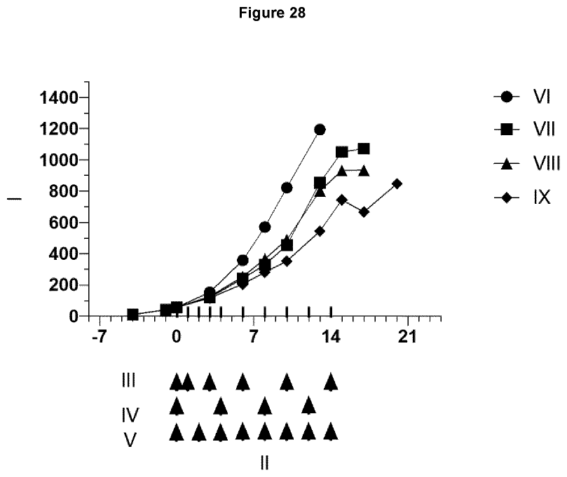

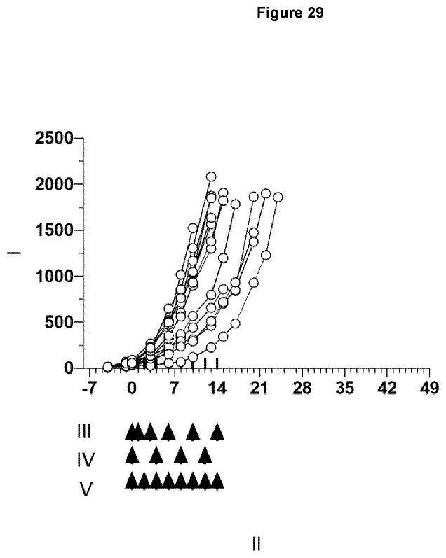

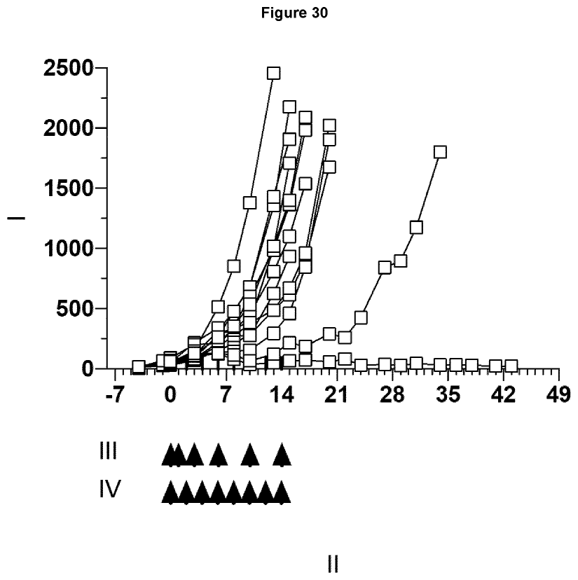

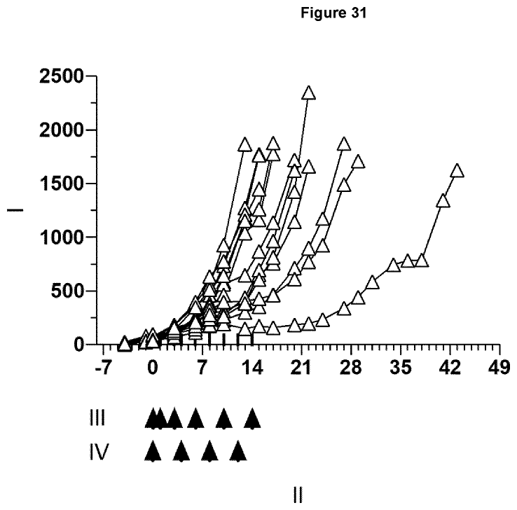

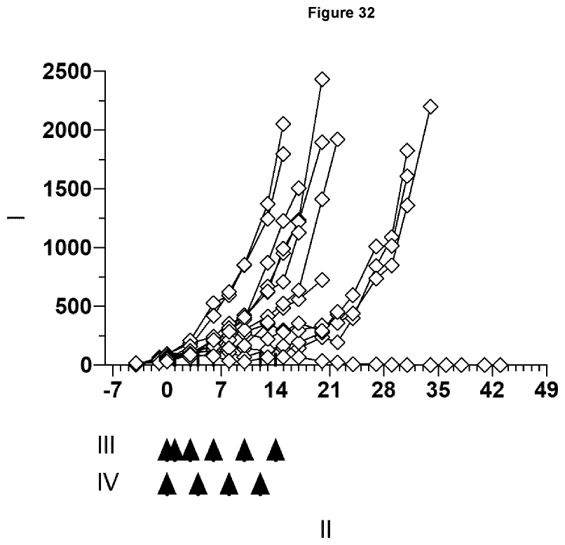

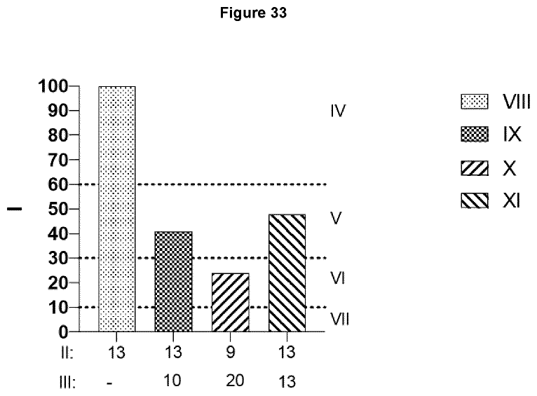

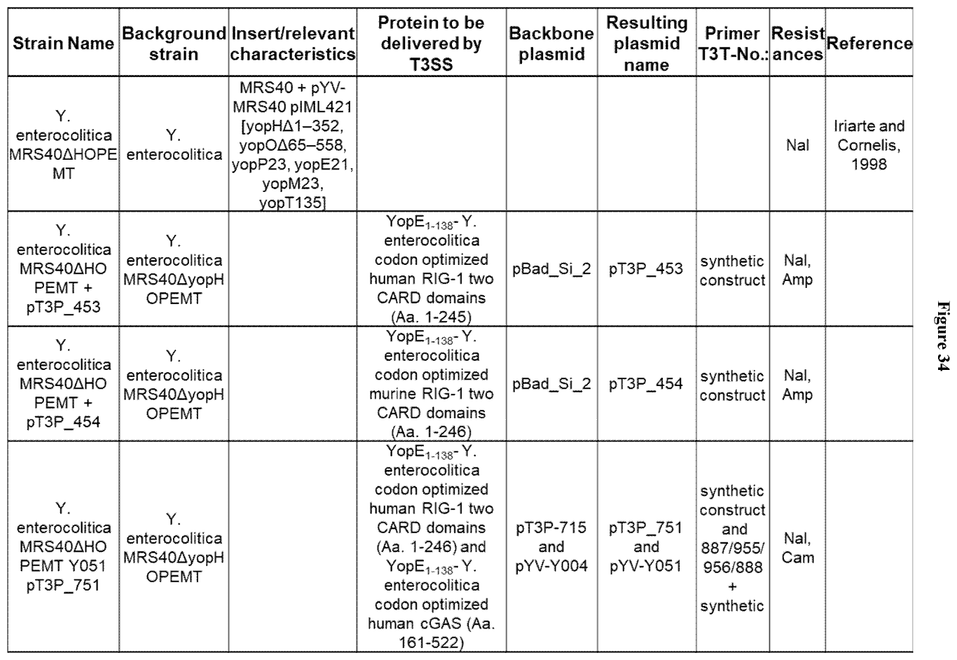

Bacterial strains and growth conditions. The strains used in this study are listed in FIG. 34. E. coli Top10 (from Invitrogen), used for plasmid purification and cloning, and E. coli Sm10λ pir (Simon et al., 1983), used for conjugation, as well as E. coli BW19610 (Metcalf et al., 1994), used to propagate pKNG101, were routinely grown on LB agar plates and in LB broth at 37° C. Ampicillin was used at a concentration of 200 μg/ml (Yersinia) or 100 μg/ml (E. coli). Streptomycin was used at a concentration of 100 μg/ml to select for suicide vectors. Chloramphenicol was used at a concentration of 10 μg/ml. Y. enterocolitica MRS40 (O:9, biotype 2) (Sarker et al., 1998) is an Ampicillin sensitive derivate of Y. enterocolitica E40 (Sory and Cornelis, 1994). All Y. enterocolitica strains were routinely grown on Brain Heart Infusion (BHI; Difco) at RT. To all Y. enterocolitica strains Nalidixic acid was added (35 μg/ml). Y. enterocolitica MRS40 and E40 both comprise the identical pYV plasmid referred to as pYV-MRS40 or pYV-E40 (the virulence plasmid of Y. enterocolitica MRS40 and E40 strains), which is shown in FIG. 1. The full sequence of the closely related pYV plasmid of Y. enterocolitica W22703, pYVe227, is accessible at Genbank (AF102990.1). The pYV plasmid derived from pYV-MRS40 through disruption of all T3SS effector proteins (yopH, yopO, yopP, yopE, yopM, yopT) is designated pYV-Y004 and the corresponding Y. enterocolitica MRS40 strains carrying pYV-Y004 are designated Y. enterocolitica MRS40 ΔHOPEMT as shown e.g. in FIG. 43. pYV-Y004 is described and depicted in FIG. 2. Insertion of human Rig-I CARD₂ domains in fusion with YopE₁₋₁₃₈ at the place of native yopE on the pYV-Y004 resulted in pYV-Y021, and additional insertion of YopE₁₋₁₃₈ (codon adapted) in fusion with human cGAS₁₆₁₋₅₂₂ at the place of yopH in pYV021 resulted in pYV-Y051. pYV-Y051 is described and depicted in FIG. 3.

Genetic manipulations of Y. enterocolitica. Genetic manipulations of Y. enterocolitica has been described (Diepold et al., 2010; Iriarte et al., 1995). Briefly, mutators for modification or deletion of genes in the pYV plasmids or on the chromosome were constructed by 2-fragment overlapping PCR using purified pYV40 plasmid or genomic DNA as template, leading to 200-250 bp of flanking sequences on both sides of the deleted or modified part of the respective gene. Alternatively, fully synthetic DNA fragments (de-novo synthesized) with 200-250 bp of flanking sequences on both sides of the deleted or modified part of the respective gene were used. Resulting fragments were cloned in pKNG101 (Kaniga et al., 1991) in E. coli BW19610 (Metcalf et al., 1994). Sequence verified plasmids were transformed into E. coli Sm10λ pir, from where plasmids were mobilized into the corresponding Y. enterocolitica strain. Mutants carrying the integrated vector were propagated for several generations without selection pressure. Then sucrose was used to select for clones that have lost the vector. Finally, mutants were identified by colony PCR. Specific mutators (pT3P-456 and pT3P-714) are listed in Table III.

Construction of plasmids. Plasmid pBad_Si_2 and pT3T-715 were used for cloning of fusion proteins with the N-terminal 138 amino acids of YopE (SEQ ID NO: 25). pBad_Si_2 (FIG. 4) was constructed by cloning of the SycE-YopE1-138 fragment containing endogenous promoters for YopE and SycE from purified pYV40 into KpnI/HindIII site of pBad-MycHisA (Invitrogen). Additional modifications include removal of the NcoI/BglII fragment of pBad-MycHisA by digestion, Klenow fragment treatment and religation. Further at the 3′ end of YopE₁₋₁₃₈ the following cleavage sites were added: XbaI-XhoI-BstBI-HindIII (creating an MCS, see SEQ ID NO: 36). It features a pBR322 origin of replication (SEQ ID NO: 29) Vectors pT3P-454 (FIG. 5) and pT3P-453 (FIG. 6) derivate from pBAD_SI_2 by cloning murine or human Rig1-CARD₂ domains (respectively) using restriction/ligation technique in the XbaI/HindIII sites, in frame with YopE 1-138 ORF, as described below.

pT3P-715 (FIG. 7) is a fully synthetic plasmid (de-novo synthesized vector) with similar characteristics to pBAD_Si_2, while the corresponding AraC coding region has been deleted, and the ampicillin resistance gene (plus 70 bp upstream) is replaced by a chloramphenicol resistance gene with 200 bp upstream region. For clarity, pT3P-715 comprises the SycE-YopE1-138 fragment containing endogenous promoters for YopE and SycE from pYV40, where at the 3′ end of YopE1-138 the following cleavage sites were added: XbaI-XhoI-BstBI-HindIII. It features a pBR322 origin of replication, and a chloramphenicol acetyl transferase (cat) from transposable genetic element Tn9 (Alton and Vapnek, 1979). pBad_Si_2 and pT3P-715 are medium copy number plasmids with a pBR322 (pMB1) origin of replication (SEQ ID NO: 29).

Vector pT3P-751 (FIG. 8) derivate from pT3P-715 by cloning as one operon YopE₁₋₁₃₈ in fusion with human Rig1-CARD2 domains, followed by YopE₁₋₁₃₈ (codon adapted) in fusion with human cGAS₁₆₁₋₅₂₂ using restriction/ligation technique in the XbaI/HindIII sites, as described below.

Heterologous proteins for delivery—RIG-I. RIG-I (also called DDX58; Uniprot Q6Q899 for the murine protein and Uniprot 095786 for the human protein) is a cytoplasmic sensor for short double-stranded RNA and a major pattern recognition receptor of the innate immune system. RIG-I consists of an RNA helicase domain, a C-terminal domain and an N-terminal domain, which is composed of two CARD domains (Brisse and Ly, 2019). Heterologous protein for delivery was selected to be mainly composed of the N-terminal CARD domains of RIG-I alone (without the rest of the protein; human RIG-I₁₋₂₄₅: murine RIG-I₁₋₂₄₆), results in RNA-independent, constitutive activation of the RIG-I pathway. The bacterially delivered RIG-I CARD domains are accessible and result in MAVS and TBK1 activation. This is followed by nuclear translocation of activated IRF3 and IRF7, which results in transcription of ISRE-regulated coding sequences, such as IFN a and b.

Similarly, CARD domain or CARD domains of MAVS or MDA5 have been selected to function agonist-independently upon delivery by bacteria.

Heterologous proteins for delivery—cGAS. Cyclic GMP-AMP synthase (cGAS; Uniprot Q8N884 for the human protein) is a cytoplasmic sensor for DNA. cGAS is a nucleotidyltransferase that catalyses the formation of cyclic GMP-AMP (cGAMP) from ATP and guanosine triphosphate (GTP), and is part of the cGAS-STING DNA sensing pathway. It has two major dsDNA-binding sites on opposite sides of a catalytic pocket and is activated by binding to cytosolic DNA. After binding to DNA, cGAS catalyses cGAMP synthesis, which then functions as a second messenger that binds to and activates the endoplasmic reticulum-located transmembrane protein 173 (TMEM173)/STING. STING then activates the protein kinases IκB kinase (IKK) and TBK1, which in turn activate the transcription factors NF-κB and IRF3 to induce interferons and other cytokines. The second messenger cGAMP may also pass to other cells in several ways and thereby pass on the danger signal of cytosolic DNA to surrounding cells. N-terminally truncated cGAS (as human cGAS₁₆₁₋₅₂₂), lacks the N-terminal DNA binding domain but retains enzymatic activity. Delivery of this truncated cGAS into eukaryotic cells leads to intracellular cGAMP production due to the enzymatic activity of cGAS, which results in activation of the STING pathway. As seen with the RIG-I pathway, activation of the STING pathway ultimately results in production of type I IFNs.

Murine and human genes were de novo synthesized, which allowed codon usage to be adapted to Y. enterocolitica (FIG. 34) and cloned as fusions to YopE1-138 into plasmids pBad_Si_2 or pT3P-715, both being medium copy-number vectors (see Table II below). Ligated plasmids were cloned in E. coli Top10. Sequenced plasmids were electroporated into the desired Y. enterocolitica strain using settings as for standard E. coli electroporation.

TABLE I
(Primer No. T3P_: Sequence)

SEQ ID NO: 32: Primer No.: prT3T_887
cacatgtggtcgacGAATAGACAGCGAAAGTTGTTGAAATAATTG
SEQ ID NO: 33: Primer No.: prT3T_955
cactacccccttgtttttatccataTTAATTGCGCGGTTTAAACGGG
SEQ ID NO: 34: Primer No.: prT3T_956
TATGGATAAAAACAAGGGGGTAGTG
SEQ ID NO: 35: Primer No.: prT3T_888
catgcgaatgggcccGTTTTCAGTATAAAAAGCACGGTATATAC

TABLE II
Cloned fusion proteins

| | Protein | | Resulting | | Primer |
|---|---|---|---|---|---|
| Protein to be | SEQ ID | Backbone | plasmid | Primers. | SEQ |
| delivred by T3SS | NO: | plasmid | name | T3T_Nr.: | ID NO: |
| YopE₁₋₁₃₈- Y. | 1 | pBad_Si_2 | pT3P-453 | synthetic | / |
| enterocolitica codon | | | | construct | |
| optimized human | | | | | |
| RIG-1 CARD₂ | | | | | |
| (Aa. 1-245) | | | | | |
| YopE₁₋₁₃₈- Y. | 4 | pBad_Si_2 | pT3P-454 | synthetic | / |
| enterocolitica codon | | | | construct | |
| optimized murine | | | | | |
| RIG-1 CARD₂ | | | | | |
| (Aa. 1-246) | | | | | |
| YopE₁₋₁₃₈- Y. | 1 and 10 | pT3P-715 | pT3P-751 | synthetic | / |
| enterocolitica codon | | | | construct | |
| optimized human | | | | | |
| RIG-1 CARD₂ | | | | | |
| (Aa. 1-245) and | | | | | |
| YopE1-138 | | | | | |
| (codon adapted)- Y. | | | | | |
| enterocolitica codon | | | | | |
| optimized human | | | | | |
| cGAS (Aa. 161-522) | | | | | |

TABLE III
Mutators for genetic modification and resulting pYV plasmids

| | To be | Back | Resulting | Resulting | Primers | Primers | | |
|---|---|---|---|---|---|---|---|---|
| Mutator/ | inserted | bone | mutator | pYV | prT3T_{—} | SEQ ID | | used with |
| Construct | onto: | plasmid | name | name | No.: | NO: | Template | parent strain |
| YopE₁₋₁₃₈- | pYV | pKNG101 | pT3P_456 | pYV-021 | PCR1: | 32/33, | PCR1: | Y. |
| Y. | (yopE::) | | | | 887/955; | 34/35, | pT3P- | enterocolitica |
| enterocolitica | | | | | PCR2: | 32/35 | 453 | ΔyopH |
| codon | | | | | 956/888; | | PCR2: | OPEMT |
| optimized | | | | | overlapping | | Y. | pYV-Y004 |
| human | | | | | PCR: | | enterocolitica | |
| RIG-1 | | | | | 887/888 | | | |
| CARD₂ | | | | | | | | |
| (Aa. 1-245) | | | | | | | | |
| YopE₁₋₁₃₈- | pYV | pKNG101 | pT3P_714 | pYV-051 | / | / | / | Y. |
| Y. | (yopE:: | | | | (Synthetic | | | enterocolitica |
| enterocolitica | and | | | | gene) | | | ΔyopH |
| codon | yopH::) | | | | | | | OPEMT |
| optimized | | | | | | | | pYV-021 |
| human | | | | | | | | |
| RIG-1 | | | | | | | | |
| CARD₂ | | | | | | | | |
| (Aa. 1-245) | | | | | | | | |
| AND | | | | | | | | |
| YopE1- | | | | | | | | |
| 138- Y. | | | | | | | | |
| enterocolitica | | | | | | | | |
| codon | | | | | | | | |
| optimized | | | | | | | | |
| human | | | | | | | | |
| cGAS | | | | | | | | |
| (Aa. 161- | | | | | | | | |
| 522) | | | | | | | | |

Bacterial Preparation for i.t./i.v. Injection to Animals

Y. enterocolitica frozen stock (7% of DMSO stock of bacteria grown to OD₆₀₀=8 and kept at −80° C.) were diluted in fresh BHI supplemented with appropriate antibiotics to an OD₆₀₀ of 0.1 and grown for ⅔h at RT before a temperature shift in a 37° C. waterbath shaker for further 2h. Finally, the bacteria were collected by centrifugation (6000 rcf, 30 sec), washed twice in PBS (sterile, endotoxin free), and resuspended in sterile PBS. The concentration of the bacterial suspension was adapted based on the OD₆₀₀, and the suspension was injected to mice according to the indications below. The inoculum administered to the mice was validated by dilution plating.

Efficacy in EMT-6 Tumor Allograft Mouse Models and i.t./i.p. Treatments

Animal housing and experimental procedures was conducted according to the French and European Regulations and the National Research Council Guide for the Care and Use of Laboratory Animals. All procedures using animals (including surgery, anesthesia and euthanasia as applicable) were approved by French authorities (CNREEA). 5-7 weeks old BALB/cByJ (EMT-6 model) mice were ordered from Charles Rivers and kept in Specific Pathogen Free (SPF) environment. After at least 5-14 days of accommodation, mice were anesthetized using isoflurane and 200 ul EMT-6 cells (1×10⁶ cells) were subcutaneously allografted into the flank of mice. Throughout the experiment, mice were scored for behavior and physical appearance, as well as body weight was measured.

Once tumors had reached a volume of 60-130 mm³ (average size of 92 mm³+/−19), mice were randomized into groups and were administered with anti-PD-1 antibody (clone: RMP1-14, catalog: BE0146, isotype: Rat IgG2a, Bioxcell) diluted in PBS to a concentration of 1 mg/ml (10 mg/kg) through i.p. injection. As control, mice were injected with sterile endotoxin-free PBS only. The same day, mice were infected with the Y. enterocolitica ΔHOPEMT encoding YopE₁₋₁₃₈-murine RIG-I CARD₂ (7.5×10⁷ bacteria) by direct injection (i.t.) into the tumour. The inoculum administered to the mice was validated by dilution plating. As control, mice were injected with sterile endotoxin-free PBS only. The day of first treatment is defined as day 0. Treatments were further repeated as described above: i.t. administration of Y. enterocolitica ΔHOPEMT encoding YopE₁₋₁₃₈-murine RIG-I CARD₂ or PBS control was performed on days 0, 1, 5, 6, 10, 11; and i.p. administration of anti-PD-1 antibody (clone: RPM1-14, isotype: Rat IgG2a, Bioxcell, 10 mg/kg per injection) or PBS control was performed on days 0, 4, 7, 11. 24 hours before the last bacterial treatment mice were administered an 8 mg/ml desferal solution (10 ml/kg) through i.p. injection. Tumor progression was followed by measurements of tumor length and width with calipers. Tumor volume was determined as 0.5×length×width². A tumor volume exceeding 1500 mm³ was defined as humane endpoint. On respective days postinfection, mice were scarified by over dosage on gas anesthesia (isoflurane) followed by cervical dislocation or exsanguination.

For re-challenge assay, on d66, 1×10⁶ EMT6 cells were injected subcutaneously in 200 μL of RPMI 1640 into the contralateral flank of all surviving mice whose first EMT6 tumors were either non-detectable (0 mm³), or smaller than 25 mm. Ten naïve mice were also grafted in the same manner in order to serve as a control for tumor growth. Tumor progression was followed as described above.

Efficacy in B16F10 Tumor Allograft Mouse Models and i.t./i.p. Treatments

Animal housing and experimental procedures were realized according to the French and European Regulations and NRC Guide for the Care and Use of Laboratory Animals. All procedures using animals were agreed by French authorities (CNREEA). 7 weeks old C57BL/6J (B16F10 model) mice were ordered from Janvier Labs and kept in Specific Pathogen Free (SPF) environment. After at least 5-14 days of accommodation, mice were anesthetized using isoflurane and 200 ul B16F10 cells (1×10⁶ cells) were subcutaneously allografted into the flank of mice. Throughout the experiment, mice were scored for behavior and physical appearance, as well as body weight was measured.

Once tumors had reached a volume of 30-120 mm³ (average size of 71 mm³+/−25), mice were randomized into groups, and were administered an anti-PD-1 antibody (clone: RMP1-14, catalog: BE0146, isotype: Rat IgG2a, Bioxcell) diluted in sterile PBS to a concentration of 1 mg/mL (10 mg/kg) through i.p. injection. As control, mice were injected with sterile endotoxin-free PBS only. On the same day, mice were infected with Y. enterocolitica ΔHOPEMT encoding YopE₁₋₁₃₈-murine RIG-I CARD₂ (7.5×10⁷ bacteria) by direct injection (i.t.) into the tumour. The inoculum administered to the mice was validated by dilution plating. As control, mice were injected with sterile endotoxin-free PBS only. The day of first treatment is defined as day 0. Treatments were further repeated as described above: i.t. administration of Y. enterocolitica ΔHOPEMT encoding YopE₁₋₁₃₈-murine RIG-I CARD₂ or PBS control was performed on days 0, 1, 2, 3, 6, 9; and i.p. administration of anti-PD-1 antibody or PBS control was performed on days 0, 4, 7, 11. 24 hours before the last bacterial treatment mice were administered an 8 mg/ml desferal solution (10 ml/kg) through i.p. injection. Tumor progression was followed by measurements of tumor length and width with calipers. Tumor volume was determined as 0.5×length×width². A tumor volume exceeding 1500 mm³ was defined as humane endpoint. On respective days postinfection, mice were sacrificed by gas anesthesia over-dosage (isoflurane) followed by cervical dislocation or exsanguination.

Efficacy in B16F10 Tumor Allograft Mouse Models and i.v./i.p. Treatments

All animal experiments described in this study were reviewed and approved by the local ethic committee (CELEAG, Comité d'éthique Local pour l'expérimentation animale Genevois). 8 weeks old C57BL/6J (B16F10 model) mice were ordered from Charles Rivers Labs. After at least five days of accommodation, mice were anesthetized using isoflurane and 100 ul of B16F10 cells (5×10⁵ cells) were subcutaneously allografted into the flank of mice. Throughout the experiment, mice were scored for behavior and physical appearance, as well as body weight was measured.

Once tumors had reached a volume of 40-120 mm³ (average size of 66 mm³+/−22), mice were randomized into groups and were administered a desferal solution (10 ml/kg) through i.p. injection, this corresponds to day 0. On the same day, mice were infected with Y. enterocolitica ΔHOPEMT encoding YopE₁₋₁₃₈-murine RIG-I CARD₂ (1×10⁷ bacteria) by injection into the tail vein on days 0, 2, 4, 6, 9, 13, 16, 20. The inoculum administered to the mice was validated by dilution plating. As control, mice were injected with sterile endotoxin-free PBS only. Anti-PD-1 or isotype control antibody were administered i.p. at a dose of 10 mg/kg, on days 0, 4, 6 and 9. In addition, all mice received an i.p. injection of desferal at a dose of 10 mL/kg at days 6, 13, 20. Tumor progression was followed by measurements of tumor length and width with manual calipers. Tumor volume was determined as 0.5×length×width². A tumor volume exceeding 1500 mm³ was defined as humane endpoint. On respective days postinfection, mice were sacrificed.

Efficacy in CT26 Tumor Allograft Mouse Models and i.t./i.p. Treatments

Animal housing and experimental procedures was conducted according to the French and European Regulations and the National Research Council Guide for the Care and Use of Laboratory Animals. All procedures using animals (including surgery, anesthesia and euthanasia as applicable) were approved by French authorities (CNREEA). 5-6 weeks old BALB/c (BALB/cByJ) mice were ordered from Charles Rivers and kept in Specific Pathogen Free (SPF) environment. After at least 5-14 days of accommodation, mice were anesthetized using isoflurane and 200 ul CT26 cells (1×10⁶ cells) were subcutaneously allografted into the flank of mice. Throughout the experiment, mice were scored for behavior and physical appearance, as well as body weight was measured.

Once tumors had reached a volume of 30-120 mm³ (average size of 58 mm³+/−19), mice were randomized into groups, and administered with anti-PD-1 antibody (ref: BE0146, BioXcell; clone: RMP1-14; isotype: Rat IgG2a) or its respective controls: rat IgG2a isotype (ref: BE0089, BioXcell; clone: 2A3) and rat IgG2b isotype (ref: BE0090, BioXcell; clone: LTF-2); at a dose of 10 mg/kg through i.p. injection. On the same day, at least four hours later, mice were infected with the Y. enterocolitica ΔHOPEMT encoding YopE₁₋₁₃₈-human RIG-I CARD₂ and YopE₁₋₁₃₈-human cGAS₁₆₁₋₅₂₂ (7.5×10⁷ bacteria) by intratumoral (i.t.) administration. The inoculum administered to the mice was validated by dilution plating. As control, mice were injected with sterile endotoxin-free PBS only. The day of first treatment is defined as day 0. Treatments were further repeated as described above: i.t. administration of Y. enterocolitica ΔHOPEMT encoding YopE₁₋₁₃₈-human RIG-I CARD₂ and YopE₁₋₁₃₈-human cGAS₁₆₁₋₅₂₂ (7.5×10⁷ bacteria) or sterile PBS control was performed on days 0, 1, 3, 6, 10, 14; i.p. injections of rat IgG2a antibodies (anti-PD-1 or control isotype) was performed at 10 mg/kg on days 0, 4, 8 and 12, while also an IgG2b isotype control was included if indicated at 10 mg/kg on days 0, 2, 4, 6, 8, 10, 12, 14. On day 0, 7 and 14, about an hour before injection of bacteria or sterile PBS control, mice were administered an 8 mg/ml desferal solution (10 ml/kg) through i.p. injection. Tumor progression was followed by measurements of tumor length and width with calipers. Tumor volume was determined as 0.5×length×width². A tumor volume exceeding 1500 mm³ was defined as humane endpoint. On respective days postinfection, mice were scarified by over dosage on gas anesthesia (isoflurane) followed by cervical dislocation or exsanguination.

Cell culture, bacterial preparation and infection for in vitro assay. B16 Blue ISG cells (purchased from InvivoGen) were cultured in RPMI 1640 supplemented with 10% FCS and 2 mM L-Glutamine. Y. enterocolitica frozen stock (7% of DMSO stock of bacteria grown to OD₆₀₀=8 and kept at −80° C.) were diluted in fresh BHI to an OD600 of 0.1 and grown for 2h at RT on an orbital shaker (150 rpm) before a temperature shift in a 37° ° C. waterbath shaker (150 rpm) for further 1h. Finally, the bacteria were collected by centrifugation (6000 rcf, 60 sec), washed once with DMEM supplemented with 10 mM HEPES and 2 mM L-glutamine, and resuspended in the same medium. The concentration of the bacterial suspension was adjusted based on OD₆₀₀. Cells seeded in 96-well plates were infected at indicated MOIs, plates were centrifuged for 1 min at 500 g and placed at 37° ° C./5% CO₂ for indicated time periods.

Direct type I Interferon activation assay. Murine B16F1 melanoma cells stably expressing secreted embryonic alkaline phosphatase (SEAP) under the control of the I-ISG54 promoter, which is comprised of the IFN-inducible ISG54 promoter enhanced by a multimeric ISRE, were purchased from InvivoGen (B16-Blue ISG). Growth conditions and type I IFN assay were adapted from the protocols provided by InvivoGen. Briefly, 15,000 B16-Blue ISG cells in 150 microliter test medium (RPMI+2 mM L-glutamine+10% FCS) per well were seeded in a flat-bottom 96-well plate (NUNC or Corning). The next day, the cells were infected with the bacterial strains to be assessed by adding 15 microliter per well of the desired multiplicity of infection (MOI) followed by a brief centrifugation (500 g, 60 sec, RT). After 2 hours of incubation (37° C. and 5% CO₂) the bacteria were killed by adding test medium containing penicillin (100 U/ml) and streptomycin (100 ug/ml) for 2 hours in the incubator (37° C. and 5% CO₂). The supernatant was then removed, cells were washed with sterile PBS, and 100 uL of fresh test medium containing penicillin/streptomycin was added. The incubation was continued for 16h. Detection of SEAP followed the QUANTI-Blue™ (InvivoGen) recommendations: 20 ul of the cell supernatant was incubated with 180 ul detection reagent (QUANTI-Blue™, InvivoGen). The plate was incubated at 37° C. and SEAP activity was measured by reading the OD at 650 nm using a microplate reader (Molecular Devices).

B) Results

A Combination of Anti-PD-1 Checkpoint Inhibitor (i.p.) with Yersinia enterocolitica ΔHOPEMT Encoding mRig1-CARD₂ (i.t.) Treatment Promotes Complete Tumour Regression in Balb/C Mice Allografted with EMT-6 (Breast Cancer Model) Cells.

Wildtype Balb/c mice allografted s.c. with EMT-6 breast cancer cells were intratumorally (i.t.) injected with sterile PBS, or with 7.5×10⁷ CFU (colony forming units) of Y. enterocolitica ΔHOPEMT encoding YopE₁₋₁₃₈-murine RIG-I CARD₂ (RIG-I₁₋₂₄₆) on a medium copy number vector (whereon YopE₁₋₁₃₈-RIG-I CARD₂ is encoded under control of the yopE promoter). In combination, mice were intraperitoneally (i.p.) injected with anti-PD-1 antibody (clone: RPM1-14, isotype: Rat IgG2a, Bioxcell, 10 mg/kg per injection) or with sterile PBS as control. Groups were distributed as (indicated as i.t. treatment+i.p. treatment): PBS+PBS; Y. enterocolitica ΔHOPEMT encoding YopE₁₋₁₃₈-murine RIG-I CARD₂+PBS; PBS+anti-PD-1; Y. enterocolitica ΔHOPEMT encoding YopE₁₋₁₃₈-murine RIG-I CARD₂+anti-PD-1, (15 mice/group). Once tumors had reached a volume of 60-130 mm³ (average size of 92 mm³+/−19), mice were randomized into groups and treatment started (the day of the first treatment is defined as day 0). i.t. treatments were performed on d0, d1, d5, d6, d10 and d11 and i.p. treatments were performed on d0, d4, d7, and d11. Tumor volume was measured over the following days with calipers.

Upon treatment, the average tumour size of the group treated with the combination of anti-PD-1 and Y. enterocolitica ΔHOPEMT encoding YopE₁₋₁₃₈-murine RIG-I CARD₂ showed a significantly more pronounced tumour growth reduction when compared to each treatment alone (FIG. 9). Also, the control group (PBS/PBS) showed no tumour regression (FIG. 10), while administration of anti-PD-1 CPI alone led to 1 complete regression (FIG. 12); treatment with Y. enterocolitica ΔHOPEMT encoding YopE₁₋₁₃₈-murine RIG-I CARD₂ alone led to 4 complete regressions (FIG. 11); and co-administration of anti-PD-1 CPI and Y. enterocolitica ΔHOPEMT encoding YopE₁₋₁₃₈-murine RIG-I CARD₂ resulted in 7 complete regressions (FIG. 13).

Moreover, Kaplan-Meier survival plots (FIG. 14) show that no mice of the control group (PBS/PBS) survived until 66 days after first treatment. In the group of mice treated with anti-PD-1 alone, 7% survived at the end of the study. In the group of mice treated with Y. enterocolitica ΔHOPEMT encoding YopE₁₋₁₃₈-murine RIG-I CARD₂ alone, 27% survived at the end of the study. In the group of mice treated with Y. enterocolitica ΔHOPEMT encoding YopE₁₋₁₃₈-murine RIG-I CARD₂ in combination with anti-PD-1, 47% survived at the end of the study.

The mice whose first EMT6 tumors were either non-detectable (0 mm³), or smaller than 25 mm³, were then challenged a second time with an EMT-6 tumour cell injection into the contralateral flank. This took place on day 66. None of the mice developed a tumour after rechallenge, whereas naïve mice injected for the first time with EMT-6 cells all developed tumours (FIG. 15).

Also, moderate but transient weight loss occurred with i.t. administration of Y. enterocolitica ΔHOPEMT encoding YopE₁₋₁₃₈-murine RIG-I CARD₂. Multiple i.t. doses did not lead to progressive weight loss. The addition of anti-PD-1 CPI to Y. enterocolitica ΔHOPEMT encoding YopE₁₋₁₃₈-murine RIG-I CARD₂ did not increase body weight loss as compared to Y. enterocolitica ΔHOPEMT encoding YopE₁₋₁₃₈-murine RIG-I CARD₂ alone.

These findings highlight that in a breast cancer model, bacteria employing a T3SS system to deliver type I IFN inducing proteins in combination with an anti-PD-1 CPI improved treatment outcome significantly (average tumour volume, number of complete regressions, probability of survival) compared to either treatment given alone, thus providing a surprising synergistic anti-tumour activity.

A Combination of Anti-PD-1 Checkpoint Inhibitors (i.p.) with Yersinia enterocolitica ΔHOPEMT Encoding mRig1-CARD₂ (i.t.) Treatment Promotes Complete Tumour Regression in C57BL/6J Mice Allografted with B16F10 Melanoma Cells.

Wildtype C57BL/6J mice allografted s.c. with B16F10 melanoma cells were intratumorally (i.t.) injected with sterile PBS, or with 7.5×10⁷ CFU of Y. enterocolitica ΔHOPEMT encoding YopE₁₋₁₃₈-murine RIG-I CARD₂ (RIG-11-246) on a medium copy number vector (whereon YopE₁₋₁₃₈-RIG-I CARD₂ is encoded under control of the yopE promoter). In combination, mice were intraperitoneally (i.p.) injected with anti-PD-1 antibody (clone: RPM1-14, isotype: Rat IgG2a, Bioxcell, 10 mg/kg per injection) or with sterile PBS as a control. Groups were distributed as (indicated as i.t. treatment+i.p. treatment): PBS+PBS; Y. enterocolitica ΔHOPEMT encoding YopE₁₋₁₃₈-murine RIG-I CARD₂+PBS; PBS+anti-PD-1; Y. enterocolitica ΔHOPEMT encoding YopE₁₋₁₃₈-murine RIG-I CARD₂+anti-PD-1 (15 mice per group).

Once tumors had reached a volume of 30-120 mm³ (average size of 71 mm³+/−25), mice were randomized into groups and treatment started (the day of the first treatment is defined as day 0). i.t. treatments were performed on d0, d1, d2, d3, d6 and d9 and i.p. treatments were performed on d0, d4, d7, d11. Tumor volume was measured over the following days with calipers.

Upon treatment, the average tumour size of the group treated with the combination of anti-PD-1 and Y. enterocolitica ΔHOPEMT encoding YopE₁₋₁₃₈-murine RIG-I CARD₂ showed a more pronounced reduction in tumour growth when compared to each treatment alone (FIG. 16). Moreover, the control group (PBS/PBS) showed no tumour regression (FIG. 17), likewise administration of anti-PD-1 alone (FIG. 19) also showed no complete tumour regression. Injection of Y. enterocolitica ΔHOPEMT encoding YopE₁₋₁₃₈-murine RIG-I CARD₂ alone led to 3 complete regressions (FIG. 18); and co-administration of anti-PD-1 CPI and Y. enterocolitica ΔHOPEMT encoding YopE₁₋₁₃₈-murine RIG-I CARD₂ resulted in 4 complete regressions (FIG. 20).

Moreover, Kaplan-Meier survival plots (FIG. 21) show that no mice of the control group (PBS/PBS) as well as those of anti-PD-1 alone, respectively, survived until study end (71 days after first treatment). In the group of mice treated with Y. enterocolitica ΔHOPEMT encoding YopE₁₋₁₃₈-murine RIG-I CARD₂ alone, 20% survived at the end of the study. In the group of mice treated with Y. enterocolitica ΔHOPEMT encoding YopE₁₋₁₃₈-murine RIG-I CARD₂ in combination with anti-PD-1 27% survived at the end of the study. Also, moderate but transient weight loss occurred with i.t. administration of Y. enterocolitica ΔHOPEMT encoding YopE₁₋₁₃₈-murine RIG-I CARD₂. Multiple i.t. doses did not lead to progressive weight loss. The addition of anti-PD-1 CPI to Y. enterocolitica ΔHOPEMT encoding YopE₁₋₁₃₈-murine RIG-I CARD₂ did not increase body weight loss as compared to Y. enterocolitica ΔHOPEMT encoding YopE₁₋₁₃₈-murine RIG-I CARD₂ alone.

These findings highlight that in a melanoma model, bacteria employing a T3SS system to deliver type I IFN inducing proteins in combination with an anti-PD-1 CPI improved treatment outcome significantly (number of complete regressions, mean tumor volume, probability of survival) compared to either treatment given alone, thus providing a surprising synergistic anti-tumour activity.

A Combination of Anti-PD-1 Checkpoint Inhibitor (i.p.) with Yersinia enterocolitica ΔHOPEMT Encoding mRig1-CARD₂ (i.v.) Treatment Promotes Complete Tumour Regression in C57BL/6J Mice Allografted with B16F10 Melanoma Cells.

Wildtype C57BL/6J mice allografted s.c. with B16F10 melanoma cells were intravenously (i.v.) injected with sterile PBS, or with 1×10⁷ CFU of Y. enterocolitica ΔHOPEMT encoding YopE₁₋₁₃₈-murine RIG-I CARD₂ (RIG-11-246) on a medium copy number vector (whereon YopE₁₋₁₃₈-RIG-I CARD₂ is encoded under control of the yopE promoter). In combination, mice were intraperitoneally (i.p.) injected with anti-PD-1 antibody or with IgG isotype control (10 mg/kg per injection). Groups were distributed as (indicated as i.v. treatment+i.p. treatment): PBS+IgG isotype control; Y. enterocolitica ΔHOPEMT encoding YopE₁₋₁₃₈-murine RIG-I CARD₂+IgG control; PBS+anti-PD-1; Y. enterocolitica ΔHOPEMT encoding YopE₁₋₁₃₈-murine RIG-I CARD₂+anti-PD-1, 15 mice per group.

Once tumors had reached a volume of 40-120 mm³ (average size of 66 mm³+/−22), mice were randomized into groups and treatment started (the day of the first treatment is defined as day 0). i.v. treatments were performed on d0, d2, d4, d6, d9, d13, d16 and d20, and i.p. treatments were performed on d0, d4, d6, d9. Tumor volume was measured over the following days with calipers.

Upon treatment, the control group (PBS/control IgG) showed no tumour regression (FIG. 22). Administration of Y. enterocolitica ΔHOPEMT encoding YopE₁₋₁₃₈-murine RIG-I CARD₂ alone or anti-PD-1 alone led to no complete tumour regression (FIGS. 23 and 24). However, co-administration of anti-PD-1 CPI and Y. enterocolitica ΔHOPEMT encoding YopE₁₋₁₃₈-murine RIG-I CARD₂ resulted in 1 complete regression (FIG. 25).

Moreover, Kaplan-Meier survival plots (FIG. 26) show that no mice of the control group (PBS/control IgG), as well as mice treated with Y. enterocolitica ΔHOPEMT encoding YopE₁₋₁₃₈-murine RIG-I CARD₂ alone and anti-PD-1 alone, survived until study end (23 days after first treatment). In the group of mice treated with Y. enterocolitica ΔHOPEMT encoding YopE₁₋₁₃₈-murine RIG-I CARD₂ in combination with anti-PD-1, 20% of mice survived at the end of the study. Also, moderate but transient weight loss occurred with i.v. administration of Y. enterocolitica ΔHOPEMT encoding YopE₁₋₁₃₈-murine RIG-I CARD₂ Multiple i.v. doses did not lead to progressive weight loss. No loss of body weight was observed with multiple i.v. doses of PBS and control IgG. The addition of anti-PD-1 CPI Y. enterocolitica ΔHOPEMT encoding YopE₁₋₁₃₈-murine RIG-I CARD₂ did not increase body weight loss as compared to Y. enterocolitica ΔHOPEMT encoding YopE₁₋₁₃₈-murine RIG-I CARD₂ alone.

These findings highlight that in a melanoma model, bacteria employing a T3SS system to deliver type I IFN inducing proteins in combination with an anti-PD-1 CPI improved treatment outcome significantly (number of complete regressions, probability of survival) compared to either treatment given alone, thus providing a surprising synergistic anti-tumour activity.

### Delivery of Human and Murine Rig1-CARD₂ Via T3SS Induces Similar Level of Type I IFN in B16F1 Melanoma Reporter Cell Line

Delivery of human RIG-I CARD₂ or murine RIG-I CARD₂ leads to induction of type I IFN signalling in B16F1 melanocytes. B16F1 IFN reporter cells were infected with Y. enterocolitica ΔHOPEMT, either a control strain not delivering a cargo, or encoding on a medium-copy number vector: YopE₁₋₁₃₈-human RIG-I CARD₂, or YopE₁₋₁₃₈-murine RIG-I CARD₂. A titration of the bacteria added to the cells was performed for each strain, indicated in as Multiplicity of Infection (MOI). IFN stimulation was assessed based on activity of secreted alkaline phosphatase which is under the control of the I-ISG54 promoter which is comprised of the IFN-inducible ISG54 promoter enhanced by a multimeric ISRE, and which is assessed by OD₆₅₀ measurement. The results show that delivery of the human version (human RIG-I amino acids 1-245) and the murine version of the Rig1-CARD₂ domains (murine RIG-I amino acids 1-246) triggered the induction of type I IFN on a similar level on murine cells (FIG. 27). Similar experiments performed on human cell lines confirmed that delivery of the human version and the murine version of the Rig1-CARD₂ domains triggered the induction of type I IFN on a similar level also in human cells.

A Combination of Anti-PD-1 Checkpoint Inhibitors (i.p.) with Yersinia enterocolitica ΔHOPEMT hRig1-CARD₂ and h-cGAS₁₆₁₋₅₂₂ (i.t.) Treatment Promotes Complete Tumour Regression in Balb/C Mice Allografted with CT26 Colon Carcinoma Cells.

Wildtype Balb/C mice allografted s.c. with CT26 colon carcinoma cells were intratumorally (i.t.) injected with sterile PBS, or with 7.5×10⁷ CFU of Y. enterocolitica ΔHOPEMT encoding YopE₁₋₁₃₈-human RIG-I CARD₂ (RIG-11-246) and YopE₁₋₁₃₈-human cGAS (cGAS₁₆₁₋₅₂₂) both on the pYV plasmid and on a medium copy number vector (whereon YopE₁₋₁₃₈-RIG-I CARD₂ and YopE₁₋₁₃₈-human cGAS₁₆₁₋₅₂₂ are encoded under control of the yopE promoter). In combination, mice were intraperitoneally (i.p.) injected with anti-PD-1 antibody (BE0146, BioXcell; clone: RMP1-14; isotype: Rat IgG2a, 10 mg/kg per injection) or with control isotypes (IgG2a control isotype: BE0089, BioXcell; clone: 2A3, and IgG2b control isotype: BE0090, BioXcell; clone: LTF-2, 10 mg/kg per injection).

Once tumors had reached a volume of 30-120 mm³ (average size of 58 mm³+/−19), mice were randomized into groups and treatment started (the day of the first treatment of is defined as day 0). i.t. administration of Y. enterocolitica ΔHOPEMT encoding YopE₁₋₁₃₈-human RIG-I CARD₂ and YopE₁₋₁₃₈-human cGAS₁₆₁₋₅₂₂ (7.5×10⁷ bacteria) or sterile PBS control was performed on days 0, 1, 3, 6, 10, 14; i.p. injections of rat IgG2a antibodies (anti-PD-1 or control isotype; while also an IgG2b isotype control was included if indicated at 10 mg/kg on days 0, 2, 4, 6, 8, 10, 12, 14) was performed at 10 mg/kg on days 0, 4, 8 and 12. Tumor volume was measured over the following days with calipers.

Groups were distributed as (indicated as i.t. treatment+i.p. treatment(s)): PBS+IgG2a+IgG2b control isotypes; Y. enterocolitica ΔHOPEMT encoding YopE₁₋₁₃₈-human RIG-I CARD₂ and YopE₁₋₁₃₈-human cGAS₁₆₁₋₅₂₂+IgG2b control isotype; PBS+anti-PD-1; Y. enterocolitica ΔHOPEMT encoding YopE₁₋₁₃₈-human RIG-I CARD₂ and YopE₁₋₁₃₈-human cGAS₁₆₁₋₅₂₂+anti-PD-1 (13 mice per group).

Upon treatment, the average tumour size of the group treated with the combination of anti-PD-1 and Y. enterocolitica ΔHOPEMT encoding YopE₁₋₁₃₈-human RIG-I CARD₂ and YopE₁₋₁₃₈-human cGAS₁₆₁₋₅₂₂ showed a more pronounced tumour growth reduction when compared to each treatment alone (FIG. 28). Moreover, the control group (PBS/IgG2a+IgG2b control isotypes, FIG. 29) and the group treated with anti-PD-1 alone (FIG. 31) showed no tumour regression, as at the end of the study (or day of sacrifice) all mice of each group harboured a tumour volume that increased more than 35% compared to their respective volume at day 0. Treatment with Y. enterocolitica ΔHOPEMT encoding YopE₁₋₁₃₈-human RIG-I CARD₂ and YopE₁₋₁₃₈-human cGAS₁₆₁₋₅₂₂ led to 1 tumour categorized as partial regression (between 50% and 95% decrease of tumour volume compared to day 0, FIG. 30). Combination treatment of anti-PD-1 CPI with Y. enterocolitica ΔHOPEMT encoding YopE₁₋₁₃₈-human RIG-I CARD₂ and YopE₁₋₁₃₈-human cGAS₁₆₁₋₅₂₂ resulted in 1 complete regression (FIG. 32). Also, the optimal tumour growth inhibition effect of the different treatments is represented in FIG. 33. Tumor growth inhibition (treated/control groups ratio, T/C, in %) is defined as the ratio of the median tumour volumes of treated groups versus median tumour volumes of animals of the control group (injected with PBS and control isotypes). The optimal value is the minimal T/C % ratio reflecting the maximal tumor growth inhibition achieved (with n>=4 animals). T/C % ratios were classified as follows: 0-10%: marked anti-tumoral activity, 10-30%: moderate anti-tumoral activity, 30-60%: marginal anti-tumoral activity, 60-100%: no anti-tumoral activity. This classification has been adapted from the literature (Johnson et al., 2001) based on internal data.

Furthermore, a moderate but transient body weight loss occurred in groups treated i.t. with Y. enterocolitica ΔHOPEMT encoding YopE₁₋₁₃₈-human RIG-I CARD₂ and YopE₁₋₁₃₈-human cGAS₁₆₁₋₅₂₂. Multiple i.t. doses did not lead to progressive weight loss, overall body weight loss was mild and transient, and mice began to recover after the treatment period. The addition of anti-PD-1 CPI to Y. enterocolitica ΔHOPEMT encoding YopE₁₋₁₃₈-human RIG-I CARD₂ and YopE₁₋₁₃₈-human cGAS₁₆₁₋₅₂₂ did not increase body weight loss as compared to Y. enterocolitica ΔHOPEMT encoding YopE₁₋₁₃₈-human RIG-I CARD₂ and YopE₁₋₁₃₈-human cGAS₁₆₁₋₅₂₂ alone.

These findings highlight that in the murine CT26 carcinoma cancer model, bacteria employing a T3SS system to deliver type I IFN inducing proteins in combination with an anti-PD-1 CPI improved treatment outcome significantly (number of complete or partial regressions, mean tumor volume, probability of survival) compared to either treatment given alone, thus providing a surprising synergistic anti-tumour activity

In summary, combinatory treatment of Y. enterocolitica ΔHOPEMT encoding YopE₁₋₁₃₈-murine RIG-I CARD₂ (RIG-I₁₋₂₄₆) (i.t. or i.v.) or Y. enterocolitica ΔHOPEMT encoding

YopE₁₋₁₃₈-human RIG-I CARD₂ and YopE₁₋₁₃₈-human cGAS₁₆₁₋₅₂₂ (i.t.) together with i.p. injection of anti-PD-1 was assessed in different murine cancer models of solid tumours, for its impact on tumour progression in different animal models of solid tumor, as well as probability of survival and relative body weight evolution. Results showed significantly improved treatment outcome compared to either treatment given alone, showing an overall surprising synergistic anti-tumour activity. Moreover, we showed that human and murine version of Rig1-CARD₂ induces similar level of type I IFN and are thus functionally equivalent.

### REFERENCES

Alto, N. M., and J. E. Dixon. 2008. Analysis of Rho-GTPase mimicry by a family of bacterial type III effector proteins. Methods Enzymol. 439:131-143.
Alto, N. M., F. Shao, C. S. Lazar, R. L. Brost, G. Chua, S. Mattoo, S. A. McMahon, P. Ghosh, T. R. Hughes, C. Boone, and J. E. Dixon. 2006. Identification of a bacterial type III effector family with G protein mimicry functions. Cell. 124:133-145.
Alton, N. K., and D. Vapnek. 1979. Nucleotide sequence analysis of the chloramphenicol resistance transposon Tn9. Nature. 282:864-869.
Brenner, D., and T. W. Mak. 2009. Mitochondrial cell death effectors. Curr Opin Cell Biol. 21:871-877.
Brisse, M., and H. Ly. 2019. Comparative Structure and Function Analysis of the RIG-I-Like Receptors: RIG-I and MDA5. Front Immunol. 10:1586.
Carretero-Gonzalez, A., D. Lora, I. Ghanem, J. Zugazagoitia, D. Castellano, J. M. Sepulveda, J. A. Lopez-Martin, L. Paz-Ares, and G. de Velasco. 2018. Analysis of response rate with ANTI PD1/PD-L1 monoclonal antibodies in advanced solid tumors: a meta-analysis of randomized clinical trials. Oncotarget. 9:8706-8715.
Chalah, A., and R. Khosravi-Far. 2008. The mitochondrial death pathway. Adv Exp Med Biol. 615:25-45.
Cornelis, G. R. 2006. The type III secretion injectisome. Nat Rev Microbiol. 4:811-825.
Diepold, A., M. Amstutz, S. Abel, I. Sorg, U. Jenal, and G. R. Cornelis. 2010. Deciphering the assembly of the Yersinia type III secretion injectisome. EMBO J. 29:1928-1940.
Eisenhauer, E. A., P. Therasse, J. Bogaerts, L. H. Schwartz, D. Sargent, R. Ford, J. Dancey, S. Arbuck, S. Gwyther, M. Mooney, L. Rubinstein, L. Shankar, L. Dodd, R. Kaplan, D. Lacombe, and J. Verweij. 2009. New response evaluation criteria in solid tumours: revised RECIST guideline (version 1.1). Eur J Cancer. 45:228-247.
Feldman, M. F., S. Muller, E. Wuest, and G. R. Cornelis. 2002. SycE allows secretion of YopE-DHFR hybrids by the Yersinia enterocolitica type III Ysc system. Mol Microbiol. 46:1183-1197.
Forsberg, A., and H. Wolf-Watz. 1990. Genetic analysis of the yopE region of Yersinia spp.: identification of a novel conserved locus, yerA, regulating yopE expression. Journal of bacteriology. 172:1547-1555.
Fuchs, Y., and H. Steller. 2011. Programmed cell death in animal development and disease. Cell. 147:742-758.
Howard, S. L., M. W. Gaunt, J. Hinds, A. A. Witney, R. Stabler, and B. W. Wren. 2006. Application of comparative phylogenomics to study the evolution of Yersinia enterocolitica and to identify genetic differences relating to pathogenicity. Journal of bacteriology. 188:3645-3653.
Iriarte, M., I. Stainier, and G. R. Cornelis. 1995. The rpoS gene from Yersinia enterocolitica and its influence on expression of virulence factors. Infect Immun. 63:1840-1847.
Isberg, R. R., D. L. Voorhis, and S. Falkow. 1987. Identification of invasin: a protein that allows enteric bacteria to penetrate cultured mammalian cells. Cell. 50:769-778.
Ittig, S., C. Schmutz, C. A. Kasper, M. Amstutz, A. Schmidt, L. Sauteur, M. A. Vigano, S. H. Low, M. Affolter, G. R. Cornelis, E. A. Nigg, and C. Arrieumerlou. 2015. A bacterial type III secretion-based protein delivery tool for broad applications in cell biology. The Journal of cell biology. 211:913-931.
Johnson, J. I., S. Decker, D. Zaharevitz, L. V. Rubinstein, J. M. Venditti, S. Schepartz, S. Kalyandrug, M. Christian, S. Arbuck, M. Hollingshead, and E. A. Sausville. 2001. Relationships between drug activity in NCI preclinical in vitro and in vivo models and early clinical trials. Br J Cancer. 84:1424-1431.
Kaniga, K., I. Delor, and G. R. Cornelis. 1991. A wide-host-range suicide vector for improving reverse genetics in gram-negative bacteria: inactivation of the blaA gene of Yersinia enterocolitica. Gene. 109:137-141.
Kranzusch, P. J., A. S. Lee, J. M. Berger, and J. A. Doudna. 2013. Structure of human cGAS reveals a conserved family of second-messenger enzymes in innate immunity. Cell Rep. 3:1362-1368.
Metcalf, W. W., W. Jiang, and B. L. Wanner. 1994. Use of the rep technique for allele replacement to construct new Escherichia coli hosts for maintenance of R6K gamma origin plasmids at different copy numbers. Gene. 138:1-7.
Mota, L. J., and G. R. Cornelis. 2005. The bacterial injection kit: type Ill secretion systems. Ann Med. 37:234-249.
Mulder, B., T. Michiels, M. Simonet, M. P. Sory, and G. Cornelis. 1989. Identification of additional virulence determinants on the pYV plasmid of Yersinia enterocolitica W227. Infect Immun. 57:2534-2541.
Neubauer, H., S. Aleksic, A. Hensel, E. J. Finke, and H. Meyer. 2000. Yersinia enterocolitica 16S rRNA gene types belong to the same genospecies but form three homology groups. Int J Med Microbiol. 290:61-64.
Pelludat, C., M. Hogardt, and J. Heesemann. 2002. Transfer of the core region genes of the Yersinia enterocolitica WA-C serotype O:8 high-pathogenicity island to Y. enterocolitica MRS40, a strain with low levels of pathogenicity, confers a yersiniabactin biosynthesis phenotype and enhanced mouse virulence. Infect Immun. 70:1832-1841.
Ramamurthi, K. S., and O. Schneewind. 2005. A synonymous mutation in Yersinia enterocolitica yopE affects the function of the YopE type III secretion signal. Journal of bacteriology. 187:707-715.
Sambrook, J. 2001. Molecular cloning: a laboratory manual. D. W. Russell, editor. Cold Spring Harbor Laboratory Press, Cold Spring Harbor, N.Y..
Sarker, M. R., C. Neyt, I. Stainier, and G. R. Cornelis. 1998. The Yersinia Yop virulon: LcrV is required for extrusion of the translocators YopB and YopD. J Bacteriol. 180:1207-1214.
Seymour, L., J. Bogaerts, A. Perrone, R. Ford, L. H. Schwartz, S. Mandrekar, N. U. Lin, S. Litiere, J. Dancey, A. Chen, F. S. Hodi, P. Therasse, O. S. Hoekstra, L. K. Shankar, J. D. Wolchok, M. Ballinger, C. Caramella, E. G. E. de Vries, and R.w. group. 2017. iRECIST: guidelines for response criteria for use in trials testing immunotherapeutics. Lancet Oncol. 18:e143-e152
Simon, R., U. Priefer, and A. Puhler. 1983. A Broad Host Range Mobilization System for Invivo Genetic-Engineering—Transposon Mutagenesis in Gram-Negative Bacteria. Bio-Technol. 1:784-791.
Skurnik, M., and H. Wolf-Watz. 1989. Analysis of the yopA gene encoding the Yop1 virulence determinants of Yersinia spp. Mol Microbiol. 3:517-529.
Sory, M. P., and G. R. Cornelis. 1994. Translocation of a hybrid YopE-adenylate cyclase from Yersinia enterocolitica into Hela cells. Mol Microbiol. 14:583-594.
Suzuki, S., T. Ishida, K. Yoshikawa, and R. Ueda. 2016. Current status of immunotherapy. Jpn J Clin Oncol. 46:191-203.
Thomson, N. R., S. Howard, B. W. Wren, M. T. Holden, L. Crossman, G. L. Challis, C. Churcher, K. Mungall, K. Brooks, T. Chillingworth, T. Feltwell, Z. Abdellah, H. Hauser, K. Jagels, M. Maddison, S. Moule, M. Sanders, S. Whitehead, M. A. Quail, G. Dougan, J. Parkhill, and M. B. Prentice. 2006. The complete genome sequence and comparative genome analysis of the high pathogenicity Yersinia enterocolitica strain 8081. PLOS Genet. 2:e206.
Trosky, J. E., A. D. Liverman, and K. Orth. 2008. Yersinia outer proteins: Yops. Cellular microbiology. 10:557-565.
Waugh, D. S. 2011. An overview of enzymatic reagents for the removal of affinity tags. Protein Expr Purif. 80:283-293.
Wolke, S., N. Ackermann, and J. Heesemann. 2011. The Yersinia enterocolitica type 3 secretion system (T3SS) as toolbox for studying the cell biological effects of bacterial Rho GTPase modulating T3SS effector proteins. Cell Microbiol. 13:1339-1357.
Yoneda, Y., T. Semba, Y. Kaneda, R. L. Noble, Y. Matsuoka, T. Kurihara, Y. Okada, and N. Imamoto. 1992. A long synthetic peptide containing a nuclear localization signal and its flanking sequences of SV40 T-antigen directs the transport of IgM into the nucleus efficiently. Exp Cell Res. 201:313-320.

1. A pharmaceutical combination comprising:
(a) a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter;
(b) an immune checkpoint modulator (ICM), wherein the ICM is ezabenlimab; and optionally
(c) one or more pharmaceutically acceptable diluents, excipients or carriers.

2. A pharmaceutical combination according to claim 1, wherein the heterologous protein or a fragment thereof is a protein involved in induction or regulation of an interferon (IFN) response or a fragment thereof.

3. A pharmaceutical combination according to claim 1, wherein the heterologous protein or a fragment thereof is a protein involved in induction or regulation of a type I IFN response or a fragment thereof selected from the group consisting of the RIG-I-like receptor (RLR) family or a fragment thereof, other CARD domain containing proteins involved in antiviral signaling and type I IFN induction or a fragment thereof, and cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, leading to stimulation of STING, or a fragment thereof.

4. A pharmaceutical combination according to claim 1 wherein the heterologous protein or a fragment thereof is a protein involved in induction or regulation of a type I IFN response or a fragment thereof selected from the group consisting of RIG1, MDA5, LGP2, MAVS, WspR, DncV, DisA and DisA-like, CdaA, CdaS and cGAS, or a fragment thereof.

5. A pharmaceutical combination according to claim 1, wherein the recombinant Gram-negative bacterial strain is a Yersinia strain.

6. (canceled)

7. A method for the prevention, delay of progression or treatment of cancer in a subject, the method comprising administering the pharmaceutical combination according to claim 1 to the subject.

8. A method according to claim 7, wherein the method additionally comprises detection of a biomarker.

9. A kit of parts comprising a first container, a second container and a package insert, wherein the first container comprises at least one dose of a medicament comprising a recombinant Gram-negative bacterial strain which comprises a polynucleotide molecule comprising a nucleotide sequence encoding a heterologous protein or a fragment thereof fused in frame to the 3′end of a nucleotide sequence encoding a delivery signal from a bacterial effector protein, wherein the nucleotide sequence encoding the delivery signal from a bacterial effector protein is operably linked to a promoter, the second container comprises at least one dose of a medicament comprising an immune checkpoint inhibitor (ICM), wherein the ICM is ezabenlimab; and the package insert optionally comprises instructions for treating a subject for cancer using the medicaments.

10. A pharmaceutical combination according to claim 2, wherein the recombinant Gram-negative bacterial strain is a Yersinia strain.

11. A pharmaceutical combination according to claim 3, wherein the recombinant Gram-negative bacterial strain is a Yersinia strain.

12. A pharmaceutical combination according to claim 4, wherein the recombinant Gram-negative bacterial strain is a Yersinia strain.

13. The method according to claim 7, wherein the heterologous protein or a fragment thereof is a protein involved in induction or regulation of an interferon (IFN) response or a fragment thereof.

14. The method according to claim 7, wherein the heterologous protein or a fragment thereof is a protein involved in induction or regulation of a type I IFN response or a fragment thereof selected from the group consisting of the RIG-I-like receptor (RLR) family or a fragment thereof, other CARD domain containing proteins involved in antiviral signaling and type I IFN induction or a fragment thereof, and cyclic dinucleotide generating enzymes such as cyclic-di-AMP cyclases, cyclic-di-GMP cyclases and cyclic-di-GAMP cyclases selected from the group consisting of WspR, DncV, DisA and DisA-like, CdaA, CdaS and cGAS, leading to stimulation of STING, or a fragment thereof.

15. The method according to claim 7, wherein the heterologous protein or a fragment thereof is a protein involved in induction or regulation of a type I IFN response or a fragment thereof selected from the group consisting of RIG1, MDA5, LGP2, MAVS, WspR, DncV, DisA and DisA-like, CdaA, CdaS and cGAS, or a fragment thereof.

16. The method according to claim 7, wherein the recombinant Gram-negative bacterial strain is a Yersinia strain.
